SEQUENCE LISTING (101 sequences)

SEQ ID NO: 1 - protein, 548 aa, Escherichia coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 2 - DNA, 1644 bp, artificial sequence
  coding region codon optimized for expression in Saccharomyces cerevisiae
atggctgctaaagatgtaaagttcggtaatgatgctagagtaaaaatgttgagaggtgtaaatgtattggctgacgctgtaaaagtaactttgggtccaaaaggtagaaatgttgtcttggataagtcttttggtgctcctaccataactaaagacggtgtttcagtcgcaagagaaatcgaattggaggataagttcgaaaacatgggtgctcaaatggtcaaagaagtcgcctctaaggctaacgatgctgcaggtgacggtactacaaccgctactgttttggctcaagcaattataacagaaggtttaaaagcagttgccgctggtatgaatccaatggatttgaaaagaggtattgacaaggccgtcactgcagccgtagaagaattgaaagcattatcagtcccttgttctgattcaaaggccatcgctcaagtaggtaccatttccgctaacagtgatgaaactgttggtaaattaattgcagaagccatggacaaagtcggtaaagaaggtgtaataaccgttgaagatggtactggtttgcaagatgaattagacgtagttgagggtatgcaatttgatagaggttatttgtcaccatacttcatcaataagcctgaaacaggtgctgttgaattggaatccccttttattttgttggcagataaaaagattagtaacataagagaaatgttgccagttttagaagctgtcgcaaaagccggtaaacctttgttaatcattgctgaagatgttgaaggtgaagcattggcaacattagtcgtaaataccatgagaggtattgtaaaagttgctgcagttaaggctccaggtttcggtgacagaagaaaagctatgttgcaagacattgcaacattaaccggtggtacagttatctccgaagaaattggtatggaattggaaaaggccaccttggaagatttgggtcaagctaagagagttgtcattaataaggatactacaaccatcatcgacggtgtaggtgaagaagccgctatacaaggtagagttgctcaaataagacaacaaatcgaagaagcaacttctgattatgacagagaaaaattgcaagaaagagttgcaaagttagccggtggtgtcgctgtaattaaagttggtgcagccaccgaagtcgaaatgaaggaaaagaaagcaagagtagaagatgctttgcatgcaacaagagctgcagttgaagaaggtgtagttgcaggtggtggtgtcgccttaattagagtagcctccaaattggctgatttgagaggtcaaaatgaagaccaaaacgtaggtatcaaggttgccttaagagctatggaagcaccattgagacaaatcgttttgaactgtggtgaagaacctagtgtcgtagctaacactgttaaaggtggtgacggtaattatggttacaacgccgctacagaagaatacggtaacatgatcgatatgggtatattggacccaactaaggtcacaagatctgcattgcaatacgcagcctcagttgccggtttaatgattactacagaatgcatggttacagatttgcctaaaaacgacgctgccgacttgggtgccgcaggtggtatgggtggtatgggtggtatgggtggtatgatg

SEQ ID NO: 3 - protein, 550 aa, Actinoplanes missouriensis
MAKILSFSDDARHLLEHGVNTLADTVKVTLGPRGRNVVLDKKFGAPTITNDGVTIAKEIELTDPYENLGAQLVKEVATKTNDVAGDGTTTATVLAQALVREGLRNVTAGANPIGLKRGMDKASEVVSKALLAKAVEVADHKAIANVATISAQDATIGELIAEAMDRVGRDGVITVEEGSAMLTELEVTEGLQFDKGFISPNFVTDAESQEVVLEDAFILLTTQKISSIEELLPLLEKVLQAGKPLLIVAEDVEGQALSTLVVNALRKTIKVAAVKAPGFGDRRKAILQDLAIATGGELIAPELGYKLDQVGIESLGSARRIVVDKENTTIVDGGGNKADVTDRVAQIRKEIEASDSDWDREKLQERLAKLGGGIAVIKVGAATEVEMKERKHRIEDAIAATKAAVEEGTVPGGGAALAQVSKELEDNLGLTGEEAIGVSIVRKALVEPLRWIAQNAGHDGYVVVGKVGELGWGHGLNAATDEYVDLAAAGIIDPVKVTRNAVSNAVSIAALLLTTESLVVEKPAEAAPAAAGGGHGHSHGGHGHQHGPGF

SEQ ID NO: 4 - DNA, 1650 bp, artificial sequence
  coding region codon optimized for expression in Saccharomyces cerevisiae
atggctaagatcttgtccttctctgatgatgctagacacttgttggaacacggtgtcaacactttggctgatactgttaaggtcactttgggtccaagaggtagaaacgttgtcttggataagaagttcggtgctccaactatcaccaacgacggtgttactatcgctaaggaaatcgaattgaccgacccatacgaaaacttgggtgctcaattggtcaaggaagttgctactaagaccaacgatgtcgctggtgacggtactactaccgctactgtcttggctcaagctttggttagagaaggtttgagaaacgttaccgctggtgctaacccaatcggtttgaagagaggtatggacaaggcttctgaagttgtctccaaggctttgttggctaaggctgtcgaagttgctgatcacaaggctatcgctaacgtcgctactatctctgctcaagacgctaccatcggtgaattgatcgctgaagctatggatagagttggtagagacggtgtcatcactgttgaagaaggttctgctatgttgactgaattggaagtcaccgaaggtttgcaattcgacaagggtttcatctctccaaacttcgttaccgatgctgaatcccaagaagttgtcttggaagacgctttcatcttgttgactacccaaaagatctcttccatcgaagaattgttgccattgttggaaaaggtcttgcaagctggtaaaccattgttgatcgtcgctgaagacgttgaaggtcaagctttgtctactttggttgtcaacgctttgagaaagaccatcaaggtcgctgctgttaaggctccaggtttcggtgacagaagaaaggctatcttgcaagacttggctatcgctactggtggtgaattgatcgctccagaattgggttacaagttggaccaagtcggtatcgaatctttgggttccgctagaagaatcgttgtcgataaggaaaacactaccatcgttgacggtggtggtaacaaggctgatgtcactgacagagttgctcaaatcagaaaggaaatcgaagcttctgactccgattgggacagagaaaagttgcaagaaagattggctaagttgggtggtggtatcgctgtcatcaaggttggtgctgctaccgaagttgaaatgaaggaaagaaagcacagaatcgaagatgctatcgctgctactaaggctgctgtcgaagaaggtactgttccaggtggtggtgctgctttggctcaagtctctaaggaattggaagacaacttgggtttgaccggtgaagaagctatcggtgtctccatcgttagaaaggctttggttgaaccattgagatggatcgctcaaaacgctggtcacgacggttacgttgtcgttggtaaagtcggtgaattgggttggggtcacggtttgaacgctgctactgatgaatacgttgacttggctgctgctggtatcatcgacccagtcaaggttaccagaaacgctgtctctaacgctgtttccatcgctgctttgttgttgactaccgaatctttggtcgttgaaaagccagctgaagctgctccagctgctgctggtggtggtcacggtcactcccacggtggtcacggtcaccaacacggtccaggtttc

SEQ ID NO: 5 - protein, 540 aa, Actinoplanes missouriensis
MAKIIAFDEEARRGLERGMNQLADAVKVTLGPKGRNVVLEKKWGAPTITNDGVSIAKEIELEDSYEKIGAELVKEVAKKTDDVAGDGTTTATVLAQALVREGLRNVAAGANPMALKRGIEAAVASVSEGLQQLAKDVETKEQIASTASISAGDSTVGEIIAEAMDKVGKEGVITVEESNTFGLELELTEGMRFDKGYISAYFMTDAERMEAVFDDPYILIANSKISAVKDLLPILEKVMQSGKPLVIIAEDVEGEALATLVVNKVRGTFKSVAVKAPGFGDRRKAMLEDIAILTGGAVISEEVGLKLDAADLSLLGQARKVVITKDETTVVDGAGNGEQIQGRVNQIRAEIERSDSDYDREKLQERLAKLAGGVAVIKVGAATEVELKERKHRIEDAVRNAKAAVEEGIVPGGGVALVQAGKTAFDKLDLVGDEATGANIVKVALDAPLRQIAVNAGLEGGVVVEKVRNLSAGHGLNAATGEYVDLLAAGIIDPAKVTRSALQNAASIAALFLTTEAVVADKPEKNPAPAGAPGGGDMDF

SEQ ID NO: 6 - DNA, 1620 bp, artificial sequence
  coding region codon optimized for expression in Saccharomyces cerevisiae
atggctaagatcatcgctttcgacgaagaagctagaagaggtttggaaagaggtatgaaccaattggctgacgctgttaaggtcactttgggtccaaagggtagaaacgttgtcttggaaaagaagtggggtgctccaactatcaccaacgatggtgtctctatcgctaaggaaatcgaattggaagactcctacgaaaagatcggtgctgaattggtcaaggaagttgctaagaagactgacgatgtcgctggtgacggtactactaccgctaccgtcttggctcaagctttggttagagaaggtttgagaaacgttgctgctggtgctaacccaatggctttgaagagaggtatcgaagctgctgtcgcttctgtttccgaaggtttgcaacaattggctaaggacgttgaaactaaggaacaaatcgcttctaccgcttctatctctgctggtgactccactgtcggtgaaatcatcgctgaagctatggacaaggttggtaaagaaggtgtcatcactgttgaagaatctaacaccttcggtttggaattggaattgactgaaggtatgagattcgataagggttacatctccgcttacttcatgaccgacgctgaaagaatggaagctgtcttcgacgatccatacatcttgatcgctaactctaagatctccgctgtcaaggacttgttgccaatcttggaaaaggttatgcaatctggtaaaccattggtcatcatcgctgaagacgttgaaggtgaagctttggctactttggttgtcaacaaggttagaggtactttcaagtctgtcgctgttaaggctccaggtttcggtgacagaagaaaggctatgttggaagacatcgctatcttgactggtggtgctgtcatctctgaagaagttggtttgaagttggatgctgctgacttgtccttgttgggtcaagctagaaaggttgtcatcaccaaggatgaaactaccgttgttgacggtgctggtaacggtgaacaaatccaaggtagagttaaccaaatcagagctgaaatcgaaagatctgactccgattacgacagagaaaagttgcaagaaagattggctaagttggctggtggtgtcgctgttatcaaggtcggtgctgctaccgaagttgaattgaaggaaagaaagcacagaatcgaagacgctgtcagaaacgctaaggctgctgtcgaagaaggtatcgttccaggtggtggtgtcgctttggttcaagctggtaaaactgctttcgataagttggacttggttggtgacgaagctaccggtgctaacatcgtcaaggttgctttggacgctccattgagacaaatcgctgtcaacgctggtttggaaggtggtgttgtcgttgaaaaggttagaaacttgtctgctggtcacggtttgaacgctgctactggtgaatacgtcgatttgttggctgctggtatcatcgacccagctaaggttaccagatctgctttgcaaaacgctgcttccatcgctgctttgttcttgactaccgaagctgtcgttgctgacaagccagaaaagaacccagctccagctggtgctccaggtggtggtgacatggacttc

SEQ ID NO: 7 - protein, 545 aa, Bacteroides thetaiotaomicron
MAKEILFNIDARDQLKKGVDALANAVKVTLGPKGRNVIIEKKFGAPHITKDGVTVAKEIELADAYQNTGAQLVKEVASKTGDDAGDGTTTATVLAQAIVAEGLKNVTAGASPMDIKRGIDKAVAKVVESIKAQAETVGDNYDKIEQVATVSANNDPVIGKLIADAMRKVSKDGVITIEEAKGTDTTIGVVEGMQFDRGYLSAYFVTNTEKMECEMEKPYILIYDKKISNLKDFLPILEPAVQTGRPLLVIAEDVDSEALTTLVVNRLRSQLKICAVKAPGFGDRRKEMLEDIAILTGGVVISEEKGLKLEQATIEMLGTADKVTVSKDYTTIVNGAGVKENIKERCDQIKAQIVATKSDYDREKLQERLAKLSGGVAVLYVGAASEVEMKEKKDRVDDALRATRAAIEEGIIPGGGVAYIRAIDSLEGMKGDNADETTGIGIIKRAIEEPLREIVANAGKEGAVVVQKVREGKGDFGYNARTDVYENLHAAGVVDPAKVARVALENAASIAGMFLTTECVIVEKKEDKPEMPMGAPGMGGMGGMM

SEQ ID NO: 8 - DNA, 1635 bp, Artificial sequence
  coding region codon optimized for expression in Saccharomyces cerevisiae
atggctaaggaaatcttgttcaacatcgacgctagagaccaattgaagaagggtgttgacgctttggctaacgctgttaaggttactttgggtccaaagggtagaaacgtcatcatcgaaaagaagttcggtgctccacacatcactaaggacggtgtcaccgttgctaaggaaatcgaattggctgacgcttaccaaaacactggtgctcaattggtcaaggaagttgcttctaagaccggtgacgatgctggtgacggtactactaccgctactgtcttggctcaagctatcgttgctgaaggtttgaagaacgttaccgctggtgcttctccaatggacatcaagagaggtatcgataaggctgtcgctaaggttgtcgaatccatcaaggctcaagctgaaaccgttggtgacaactacgataagatcgaacaagtcgctactgtttctgctaacaacgacccagtcatcggtaaattgatcgctgacgctatgagaaaggtctccaaggatggtgttatcactatcgaagaagctaagggtactgacactaccatcggtgttgtcgaaggtatgcaattcgacagaggttacttgtctgcttacttcgttactaacaccgaaaagatggaatgtgaaatggaaaagccatacatcttgatctacgacaagaagatctccaacttgaaggatttcttgccaatcttggaaccagctgtccaaactggtagaccattgttggtcatcgctgaagacgttgattctgaagctttgactaccttggttgtcaacagattgagatcccaattgaagatctgtgctgttaaggctccaggtttcggtgacagaagaaaggaaatgttggaagatatcgctatcttgaccggtggtgttgtcatctctgaagaaaagggtttgaagttggaacaagctactatcgaaatgttgggtactgctgacaaggtcaccgtttccaaggattacactaccatcgtcaacggtgctggtgttaaggaaaacatcaaggaaagatgtgaccaaatcaaggctcaaatcgtcgctaccaagtctgactacgatagagaaaagttgcaagaaagattggctaagttgtctggtggtgtcgctgttttgtacgtcggtgctgcttccgaagttgaaatgaaggaaaagaaggacagagttgacgatgctttgagagctactagagctgctatcgaagaaggtatcatcccaggtggtggtgttgcttacatcagagctatcgactccttggaaggtatgaagggtgacaacgctgatgaaactaccggtatcggtatcatcaagagagctatcgaagaaccattgagagaaatcgtcgctaacgctggtaaagaaggtgctgttgtcgttcaaaaggttagagaaggtaaaggtgacttcggttacaacgctagaaccgatgtttacgaaaacttgcacgctgctggtgtcgttgacccagctaaggtcgctagagttgctttggaaaacgctgcttctatcgctggtatgttcttgactaccgaatgtgtcatcgttgaaaagaaggaagacaagccagaaatgccaatgggtgctccaggtatgggtggtatgggtggtatgatg

SEQ ID NO: 9 - protein, 544 aa, Bacillus subtilis
MAKEIKFSEEARRAMLRGVDALADAVKVTLGPKGRNVVLEKKFGSPLITNDGVTIAKEIELEDAFENMGAKLVAEVASKTNDVAGDGTTTATVLAQAMIREGLKNVTAGANPVGVRKGMEQAVAVAIENLKEISKPIEGKESIAQVAAISAADEEVGSLIAEAMERVGNDGVITIEESKGFTTELEVVEGMQFDRGYASPYMVTDSDKMEAVLDNPYILITDKKITNIQEILPVLEQVVQQGKPLLLIAEDVEGEALATLVVNKLRGTFNAVAVKAPGFGDRRKAMLEDIAVLTGGEVITEDLGLDLKSTQIAQLGRASKVVVTKENTTIVEGAGETDKISARVTQIRAQVEETTSEFDREKLQERLAKLAGGVAVIKVGAATETELKERKLRIEDALNSTRAAVEEGIVSGGGTALVNVYNKVAAVEAEGDAQTGINIVLRALEEPIRQIAHNAGLEGSVIVERLKNEEIGVGFNAATGEWVNMIEKGIVDPTKVTRSALQNAASVAAMFLTTEAVVADKPEENGGGAGMPDMGGMGGMGGMM

SEQ ID NO: 10 - DNA, 1632 bp, Artificial sequence
  coding region codon optimized for expression in Saccharomyces cerevisiae
atggctaaggaaatcaagttctccgaagaagctagaagagctatgttgagaggtgtcgatgctttggctgacgctgttaaggttaccttgggtccaaagggtagaaacgttgtcttggaaaagaagttcggttctccattgatcactaacgacggtgtcaccatcgctaaggaaatcgaattggaagatgctttcgaaaacatgggtgctaagttggtcgctgaagttgcttctaagactaacgacgttgctggtgacggtactactaccgctaccgttttggctcaagctatgatcagagaaggtttgaagaacgttaccgctggtgctaacccagtcggtgttagaaagggtatggaacaagctgtcgctgttgctatcgaaaacttgaaggaaatctctaagccaatcgaaggtaaagaatccatcgctcaagtcgctgctatctctgctgctgacgaagaagttggttccttgatcgctgaagctatggaaagagtcggtaacgatggtgttatcactatcgaagaatctaagggtttcactaccgaattggaagttgtcgaaggtatgcaattcgacagaggttacgcttctccatacatggtcaccgactccgataagatggaagctgtcttggacaacccatacatcttgatcactgataagaagatcaccaacatccaagaaatcttgccagtcttggaacaagttgtccaacaaggtaaaccattgttgttgatcgctgaagacgttgaaggtgaagctttggctactttggttgtcaacaagttgagaggtactttcaacgctgtcgctgttaaggctccaggtttcggtgacagaagaaaggctatgttggaagatatcgctgtcttgactggtggtgaagttatcaccgaagacttgggtttggatttgaagtctactcaaatcgctcaattgggtagagcttccaaggttgtcgttaccaaggaaaacactaccatcgtcgaaggtgctggtgaaactgacaagatctctgctagagtcacccaaatcagagcccaagttgaagaaactacctccgaatttgacagagaaaagttgcaagaaagattggctaagttggctggtggtgtcgctgttatcaaggttggtgctgctactgaaaccgaattgaaggaaagaaagttgagaatcgaagacgctttgaactctactagagctgctgtcgaagaaggtatcgtttccggtggtggtactgctttggtcaacgtttacaacaaggtcgctgctgttgaagctgaaggtgacgctcaaactggtatcaacatcgtcttgagagctttggaagaaccaatcagacaaatcgctcacaacgctggtttggaaggttctgtcatcgttgaaagattgaagaacgaagaaatcggtgtcggtttcaacgctgctaccggtgaatgggttaacatgatcgaaaagggtatcgttgacccaactaaggttaccagatctgctttgcaaaacgctgcttccgttgctgctatgttcttgactaccgaagctgtcgttgctgacaagccagaagaaaacggtggtggtgctggtatgccagatatgggtggcatgggcggtatgggtggtatgatg

SEQ ID NO: 11 - protein, 542 aa, Ruminococcus champanellensis
MAKQIKYGEEARKALQAGIDSLADTVKITLGPKGRNVVLDKKFGAPLITNDGVTIAKEVELEDPFENMGAQLVKEVATKTNDAAGDGTTTATLLAQAMVREGMKNIAAGANPMIVKKGIQKAVDAAVNAIKANSKPVEGSADIARVGTVSSADENVGKLIAEAMEKVSTDGVITLEESKTAETYSEVVEGMQFDRGYISPYMVTDADKMEAVYDDAYILITDKKISSIQEILPLLEQVVQAGKKLVIIAEDMEGEALTTIILNNLRGTFKCAAVKAPGFGDRRKEMLKDIAILTGGEVITSELGLELKDTTIAQLGRAKQVVIQKENTIIVDGAGASEEIKARISQIRSQIETTTSDFDKEKLQERLAKLSGGVAVIKVGAATEIEMKEKKLRIEDALAATKAAVEEGIVAGGGTALINAIPAVEKLLPSLDGDEKTGAKIILKALEEPVRQIARNAGLEGSVIIDKIRRSRKVGYGFDAYNETYVDMIPAGIVDPTKVTRSALQNAASVAAMVLTTESLVADIKEENAAAAPAMPAGGMGF

SEQ ID NO: 12 - DNA, 1626 bp, Artificial sequence
  coding region codon optimized for expression in Saccharomyces cerevisiae
atggctaagcaaatcaagtacggtgaagaagctagaaaggctttgcaagctggtatcgactccttggctgacactgttaagatcactttgggtccaaagggtagaaacgttgtcttggataagaagttcggtgctccattgatcaccaacgacggtgttactatcgctaaggaagtcgaattggaagacccattcgaaaacatgggtgctcaattggttaaggaagtcgctaccaagactaacgacgctgctggtgacggtactactaccgctaccttgttggctcaagctatggttagagaaggtatgaagaacatcgctgctggtgctaacccaatgatcgtcaagaagggtatccaaaaggctgttgacgctgctgtcaacgctatcaaggctaactctaagccagttgaaggttccgctgatatcgctagagttggtactgtctcttccgctgacgaaaacgtcggtaaattgatcgctgaagctatggaaaaggtttctaccgatggtgtcatcactttggaagaatctaagaccgctgaaacttactccgaagttgtcgaaggtatgcaattcgacagaggttacatctccccatacatggttaccgacgctgataagatggaagctgtctacgacgatgcttacatcttgatcactgacaagaagatctcttccatccaagaaatcttgccattgttggaacaagttgtccaagctggtaaaaagttggttatcatcgctgaagacatggaaggtgaagctttgactaccatcatcttgaacaacttgagaggtactttcaagtgtgctgctgttaaggctccaggtttcggtgacagaagaaaggaaatgttgaaggatatcgctatcttgaccggtggtgaagtcatcacttctgaattgggtttggaattgaaggatactaccatcgctcaattgggtagagctaagcaagttgtcatccaaaaggaaaacaccatcatcgttgacggtgctggtgcttctgaagaaatcaaggctagaatctctcaaatcagatcccaaatcgaaactaccacttctgacttcgataaggaaaagttgcaagaaagattggctaagttgtccggtggtgttgctgtcatcaaggtcggtgctgctactgaaatcgaaatgaaggaaaagaagttgagaatcgaagacgctttggctgctaccaaggctgctgttgaagaaggtatcgtcgctggtggtggtactgctttgatcaacgctatcccagctgttgaaaagttgttgccatccttggacggtgacgaaaagaccggtgctaagatcatcttgaaggctttggaagaaccagtcagacaaatcgctagaaacgctggtttggaaggttctgttatcatcgacaagatcagaagatccagaaaggtcggttacggtttcgacgcttacaacgaaacttacgttgatatgatcccagctggtatcgttgacccaaccaaggtcactagatctgctttgcaaaacgctgcttccgttgctgctatggtcttgaccactgaatctttggtcgctgacatcaaggaagaaaacgctgctgctgctccagctatgccagctggtggtatgggtttc

SEQ ID NO: 13 - protein, 546 aa, Zymomonas mobilis
MAAKDVKFSRDARERILRGVDILADAVKVTLGPKGRNVVLDKAFGAPRITKDGVSVAKEIELKDKFENMGAQMLREVASKTNDLAGDGTTTATVLAQAIVREGMKSVAAGMNPMDLKRGIDLAATKVVESLRSRSKPVSDFNEVAQVGIISANGDEEVGRRIAEAMEKVGKEGVITVEEAKGFDFELDVVEGMQFDRGYLSPYFITNPEKMVAELADPYILIYEKKLSNLQSILPILESVVQSGRPLLIIAEDIEGEALATLVVNKLRGGLKVAAVKAPGFGDRRKAMLEDIAILTKGELISEDLGIKLENVTLNMLGSAKRVSITKENTTIVDGAGDQSTIKDRVEAIRSQIEATTSDYDREKLQERVAKLAGGVAVIKVGGATEVEVKERKDRVDDALHATRAAVQEGIVPGGGTALLYATKTLEGLNGVNEDQQRGIDIVRRALQAPVRQIAQNAGFDGAVVAGKLIDGNDDKIGFNAQTEKYEDLAATGVIDPTKVVRTALQDAASVAGLLITTEAAVGDLPEDKPAPAMPGGMGGMGGMDF

SEQ ID NO: 14 - DNA, 1638 bp, Artificial sequence
  coding region codon optimized for expression in Saccharomyces cerevisiae
atggctgctaaggacgttaagttctccagagacgctagagaaagaatcttgagaggtgttgacatcttggctgacgctgttaaggtcactttgggtccaaagggtagaaacgttgtcttggacaaggctttcggtgctccaagaatcaccaaggatggtgtttctgtcgctaaggaaatcgaattgaaggacaagttcgaaaacatgggtgctcaaatgttgagagaagttgcttccaagactaacgacttggctggtgacggtactactaccgctaccgttttggctcaagctatcgtcagagaaggtatgaagtctgtcgctgctggtatgaacccaatggacttgaagagaggtatcgatttggctgctaccaaggttgtcgaatctttgagatctagatccaagccagtttccgacttcaacgaagttgctcaagtcggtatcatctctgctaacggtgacgaagaagttggtagaagaatcgctgaagctatggaaaaggtcggtaaagaaggtgttatcactgtcgaagaagctaagggtttcgacttcgaattggatgttgtcgaaggtatgcaattcgacagaggttacttgtctccatacttcatcaccaacccagaaaagatggtcgctgaattggctgacccatacatcttgatctacgaaaagaagttgtctaacttgcaatccatcttgccaatcttggaatctgttgtccaatccggtagaccattgttgatcatcgctgaagacatcgaaggtgaagctttggctactttggttgtcaacaagttgagaggtggtttgaaggttgctgctgtcaaggctccaggtttcggtgacagaagaaaggctatgttggaagatatcgctatcttgaccaagggtgaattgatctctgaagacttgggtatcaagttggaaaacgttactttgaacatgttgggttctgctaagagagtttccatcaccaaggaaaacactaccatcgttgacggtgctggtgaccaatccactatcaaggacagagtcgaagctatcagatctcaaatcgaagctactacctccgactacgatagagaaaagttgcaagaaagagttgctaagttggctggtggtgttgctgtcatcaaggtcggtggtgctaccgaagttgaagtcaaggaaagaaaggacagagttgacgatgctttgcacgctactagagctgctgttcaagaaggtatcgtcccaggtggtggtactgctttgttgtacgctactaagaccttggaaggtttgaacggtgtcaacgaagaccaacaaagaggtatcgatatcgttagaagagctttgcaagctccagtcagacaaatcgctcaaaacgctggtttcgacggtgctgttgtcgctggtaaattgatcgatggtaacgacgataagatcggtttcaacgctcaaactgaaaagtacgaagacttggctgctaccggtgttatcgatccaactaaggttgtcagaaccgctttgcaagacgctgcttctgttgctggtttgttgatcactaccgaagctgctgtcggtgacttgccagaagataagccagctccagctatgccaggtggtatgggcggcatgggtggtatggacttc

SEQ ID NO: 15 - protein, 97 aa, Escherichia coli [sequence omitted]

SEQ ID NO: 16 - DNA, 291 bp, Artificial sequence
  coding region codon optimized for expression in Saccharomyces cerevisiae
atgaatattagaccattgcatgatagagttattgttaagagaaaggaagttgaaaccaaatctgcaggtggtattgttttgactggttccgctgcagctaagagtacaagaggtgaagttttggctgttggtaatggtagaattttagaaaacggtgaagttaagcctttggatgttaaggttggtgacattgttattttcaatgatggttacggtgttaagtcagaaaagattgataacgaagaagttttgatcatgtctgaatcagatatcttggcaattgttgaagca

SEQ ID NO: 17 - protein, 104 aa, Actinoplanes missouriensis
MPVTTATKVAIKPLEDRIVVQANEAETTTASGIVIPDTAKEKPQEGTVLAVGPGRIDDKGNRVPLDVKVGDVVLYSKYGGTEVKYAGEEYLVLSARDVLAVIEK

SEQ ID NO: 18 - DNA, 312 bp, Artificial sequence
  coding region codon optimized for expression in Saccharomyces cerevisiae
atgccagtcaccaccgctactaaggtcgctatcaagccattggaagacagaatcgttgttcaagctaacgaagctgaaaccactaccgcttctggtatcgttatcccagacaccgctaaggaaaagccacaagaaggtactgttttggctgtcggtccaggtagaatcgacgataagggtaacagagtcccattggacgttaaggtcggtgacgttgtcttgtactctaagtacggtggtactgaagtcaagtacgctggtgaagaatacttggtcttgtccgctagagatgttttggctgtcatcgaaaag

SEQ ID NO: 19 - protein, 112 aa, Actinoplanes missouriensis
MSADTRTDAGLPIRMLHDRVLVRQDGGEGERRSSAGIVIPATATIGRRLSWAVAVGVGPNVRSIVVGDRVLFDPDDRSEVELHGKEYVLLRERDVHAVAANRVESDGTGLYL

SEQ ID NO: 20 - DNA, 336 bp, Artificial sequence
  coding region codon optimized for expression in Saccharomyces cerevisiae
atgtccgctgatactagaaccgatgctggtttgccaatcagaatgttgcacgatagagttttggtcagacaagatggtggtgaaggtgaaagaagatcttccgctggtatcgtcatcccagctaccgctactatcggtagaagattgtcttgggctgttgctgtcggtgttggtccaaacgtcagatccatcgttgtcggtgacagagttttgttcgatccagacgatagatctgaagtcgaattgcacggtaaagaatacgttttgttgagagaaagagacgttcacgctgttgctgctaacagagttgaatccgatggtactggtttgtacttg

SEQ ID NO: 21 - protein, 90 aa, Bacteroides thetaiotaomicron
MNIKPLADRVLILPAPAEEKTIGGIIIPDTAKEKPLKGEVVAVGHGTKDEEMVLKVGDTVLYGKYAGTELEVEGTKYLIMRQSDVLAILG

SEQ ID NO: 22 - DNA, 270 bp, Artificial sequence
  coding region codon optimized for expression in Saccharomyces cerevisiae
atgaacatcaagccattggctgacagagttttgatcttgccagctccagctgaagaaaagactatcggtggtatcatcatcccagacaccgctaaggaaaagccattgaagggtgaagttgtcgctgttggtcacggtactaaggacgaagaaatggttttgaaggtcggtgacactgttttgtacggtaaatacgctggtactgaattggaagtcgaaggtactaagtacttgatcatgagacaatctgacgttttggctatcttgggt

SEQ ID NO: 23 - protein, 94 aa, Bacillus subtilis
MLKPLGDRVVIELVESEEKTASGIVLPDSAKEKPQEGKIVAAGSGRVLESGERVALEVKEGDRIIFSKYAGTEVKYEGTEYLILRESDILAVIG

SEQ ID NO: 24 - DNA, 282 bp, Artificial sequence
  coding region codon optimized for expression in Saccharomyces cerevisiae
atgttgaagccattgggtgacagagttgttatcgaattggttgaatccgaagaaaagactgcttccggtatcgttttgccagactccgctaaggaaaagccacaagaaggtaaaatcgttgctgctggttctggtagagtcttggaatccggtgaaagagttgctttggaagtcaaggaaggtgacagaatcatcttctctaagtacgctggtactgaagtcaagtacgaaggtactgaatacttgatcttgagagaatccgatatcttggctgtcatcggt

SEQ ID NO: 25 - protein, 94 aa, Ruminococcus champanellensis
MTIKPLADRVVIKMMEAEETTKGGIILAASAQEKPQVAEIVAVGSGGVVDGKEVKMYLKVGDKVLLSKYAGTEVKLDGEDYTILRQSDILAIVE

SEQ ID NO: 26 - DNA, 282 bp, Artificial sequence
  coding region codon optimized for expression in Saccharomyces cerevisiae
atgactatcaagccattggctgacagagtcgttatcaagatgatggaagctgaagaaactactaagggtggtatcatcttggctgcttctgctcaagaaaagccacaagttgctgaaatcgttgctgtcggttccggtggtgttgttgacggtaaagaagtcaagatgtacttgaaggttggtgacaaggtcttgttgtctaagtacgctggtactgaagtcaagttggacggtgaagattacactatcttgagacaatccgacatcttggctatcgtcgaa

SEQ ID NO: 27 - protein, 95 aa, Zymomonas mobilis
MNFRPLHDRVLVRRVAAEEKTAGGIIIPDTAKEKPQEGEVIAAGNGTHSEDGKVVPLDVKAGDRVLFGKWSGTEVRVDGEDLLIMKESDILGIIS

SEQ ID NO: 28 - DNA, 285 bp, Artificial sequence
  coding region codon optimized for expression in Saccharomyces cerevisiae
atgaacttcagaccattgcacgacagagttttggttagaagagtcgctgctgaagaaaagaccgctggtggtatcatcatcccagataccgctaaggaaaagccacaagaaggtgaagttatcgctgctggtaacggtactcactctgaagacggtaaagttgtcccattggacgttaaggctggtgacagagtcttgttcggtaaatggtccggtactgaagttagagttgacggtgaagatttgttgatcatgaaggaatctgatatcttgggtatcatctcc

SEQ ID NO: 29 - protein, 394 aa, Actinoplanes missouriensis
MSVQATREDKFSFGLWTVGWQARDAFGDATRTALDPVEAVHKLAEIGAYGITFHDDDLVPFGSDAQTRDGIIAGFKKALDETGLIVPMVTTNLFTHPVFKDGGFTSNDRSVRRYAIRKVLRQMDLGAELGAKTLVLWGGREGAEYDSAKDVSAALDRYREALNLLAQYSEDRGYGLRFAIEPKPNEPRGDILLPTAGHAIAFVQELERPELFGINPETGHEQMSNLNFTQGIAQALWHKKLFHIDLNGQHGPKFDQDLVFGHGDLLNAFSLVDLLENGPDGAPAYDGPRHFDYKPSRTEDYDGVWESAKANIRMYLLLKERAKAFRADPEVQEALAASKVAELKTPTLNPGEGYAELLADRSAFEDYDADAVGAKGFGFVKLNQLAIEHLLGAR

SEQ ID NO: 30 - DNA, 1182 bp, Artificial sequence
  coding region codon optimized for expression in Saccharomyces cerevisiae
atgtccgttcaagccacaagagaagacaagtttagtttcggtttatggactgtaggttggcaagcaagagacgcattcggtgacgcaaccagaactgccttggatccagttgaagctgtccataaattggcagaaatcggtgcctacggtattacattccacgatgacgatttggttccttttggttccgatgctcaaaccagagacggtattatagccggtttcaaaaaggctttagatgaaactggtttgatcgtaccaatggttactacaaatttgtttactcatcctgtcttcaaggacggtggttttacatctaacgatagatcagtcagaagatacgctataagaaaggtattgagacaaatggatttgggtgctgaattgggtgcaaagacattagtcttgtggggtggtagagaaggtgcagaatacgattccgccaaagacgttagtgctgcattggacagatatagagaagcattgaatttgttggcacaatactctgaagatagaggttacggtttgagatttgctatagaaccaaagcctaacgaaccaagaggtgacatattgttacctactgcaggtcatgcaatcgccttcgttcaagaattggaaagaccagaattgttcggtattaatcctgaaaccggtcacgaacaaatgtctaatttgaacttcactcaaggtattgctcaagcattatggcataaaaagttgttccacatcgatttgaacggtcaacatggtccaaaattcgaccaagatttggtatttggtcacggtgacttgttgaacgctttctcattggttgatttgttggaaaacggtccagatggtgcccctgcttatgacggtccaagacattttgattacaaaccttctagaacagaagactatgatggtgtttgggaatcagcaaaggccaacatcagaatgtacttgttgttgaaggaaagagctaaggcattcagagcagatccagaagttcaagaagccttagccgcttccaaagtcgcagaattgaagacaccaaccttaaatcctggtgaaggttacgccgaattattggctgatagaagtgcatttgaagactatgatgccgacgctgttggtgctaaaggttttggttttgtcaagttaaatcaattagcaatcgaacacttattaggtgccaga

SEQ ID NO: 31 - protein, 440 aa, Escherichia coli
MQAYFDQLDRVRYEGSKSSNPLAFRHYNPDELVLGKRMEEHLRFAACYWHTFCWNGADMFGVGAFNRPWQQPGEALALAKRKADVAFEFFHKLHVPFYCFHDVDVSPEGASLKEYINNFAQMVDVLAGKQEESGVKLLWGTANCFTNPRYGAGAATNPDPEVFSWAATQVVTAMEATHKLGGENYVLWGGREGYETLLNTDLRQEREQLGRFMQMVVEHKHKIGFQGTLLIEPKPQEPTKHQYDYDAATVYGFLKQFGLEKEIKLNIEANHATLAGHSFHHEIATAIALGLFGSVDANRGDAQLGWDTDQFPNSVEENALVMYEILKAGGFTTGGLNFDAKVRRQSTDKYDLFYGHIGAMDTMALALKIAARMIEDGELDKRIAQRYSGWNSELGQQILKGQMSLADLAKYAQEHHLSPVHQSGRQEQLENLVNHYLFDK

SEQ ID NO: 32 - DNA, 1320 bp, Artificial sequence
  coding region codon optimized for expression in Saccharomyces cerevisiae
atgcaagcctattttgaccaattagacagagtaagatacgaaggttccaagtcctccaatccattagcctttagacactacaaccctgatgaattggtattgggtaaaagaatggaagaacatttgagatttgctgcatgttattggcacactttctgctggaatggtgctgatatgtttggtgttggtgcattcaacagaccatggcaacaacctggtgaagcattggccttagctaaaagaaaggctgacgtcgcatttgaatttttccataaattgcacgtaccattctattgtttccatgatgtcgacgtatcccctgaaggtgctagtttgaaggaatacataaacaacttcgcccaaatggttgatgtcttagcaggtaaacaagaagaatctggtgttaagttgttatggggtactgctaattgctttacaaacccaagatacggtgcaggtgccgctaccaatccagatcctgaagttttctcatgggcagccacccaagttgtcactgccatggaagctacacataaattgggtggtgaaaactacgtcttgtggggtggtagagaaggttacgaaacattgttaaacaccgatttgagacaagaaagagaacaattaggtagattcatgcaaatggtagttgaacataaacacaagattggtttccaaggtactttgttaatagaaccaaaacctcaagaaccaaccaagcaccaatatgattacgacgctgcaactgtctatggtttcttgaaacaattcggtttggaaaaggaaattaagttgaacatcgaagcaaaccatgccacattagctggtcactcctttcatcacgaaatcgcaaccgccattgctttgggtttattcggtagtgttgatgcaaatagaggtgacgcccaattgggttgggatacagaccaatttcctaattccgtagaagaaaacgctttggttatgtacgaaatcttgaaggcaggtggttttactacaggtggtttgaacttcgatgctaaagttagaagacaatctactgataagtacgacttattttacggtcatattggtgctatggacacaatggcattggccttaaaaatagccgctagaatgatcgaagatggtgaattggacaagagaatcgctcaaagatattctggttggaactctgaattgggtcaacaaatcttgaagggtcaaatgtctttggcagatttggccaagtacgctcaagaacatcacttatcacctgttcatcaatcaggtagacaagaacaattagaaaacttagtcaaccattacttattcgacaaa

SEQ ID NO: 33 - protein, 445 aa, Bacillus subtilis
MAQSHSSSINYFGSANKVVYEGKDSTNPLAFKYYNPQEVIGGKTLKEHLRFSIAYWHTFTADGTDVFGAATMQRPWDHYKGMDLAKMRVEAAFEMFEKLDAPFFAFHDRDIAPEGSTLKETNQNLDMIMGMIKDYMRNSGVKLLWNTANMFTNPRFVHGAATSCNADVFAYAAAQVKKGLETAKELGAENYVFWGGREGYETLLNTDLKFELDNLARFMHMAVDYAKEIGYTGQFLIEPKPKEPTTHQYDTDAATTIAFLKQYGLDNHFKLNLEANHATLAGHTFEHELRMARVHGLLGSVDANQGHPLLGWDTDEFPTDLYSTTLAMYEILQNGGLGSGGLNFDAKVRRSSFEPDDLIYAHIAGMDAFARGLKVAHKLIEDRVFEDVIQHRYRSFTEGIGLEIIEGRANFHTLEQYALNHKSIKNESGRQEKLKAILNQYILEV

SEQ ID NO: 34 - DNA, 1335 bp, Artificial sequence
  coding region codon optimized for expression in Saccharomyces cerevisiae
atggctcaatctcattccagttcaatcaactattttggaagcgcaaacaaagtggtttacgaagggaaagattcgactaatcctttagcatttaaatattataatcctcaagaagtaatcggcggaaaaacgctgaaagagcatttgcgattttctattgcctattggcatacatttactgctgatggtacagacgtttttggagcagctacgatgcaaagaccatgggatcactataaaggcatggatctagcgaagatgagagtagaagcagcatttgagatgtttgaaaaactagatgcaccattctttgcttttcatgaccgggatattgcaccagaaggcagtacgctaaaagagacaaaccaaaatttagatatgatcatgggcatgattaaagattacatgagaaatagcggcgttaagctattatggaatacagcaaacatgtttacgaatccccgtttcgtccatggtgccgcgacttcttgcaatgcagatgtgtttgcgtatgctgcagcacaagtgaaaaaagggttagaaacagcaaaagagcttggcgctgagaactatgtattttggggcggccgtgaaggatatgaaacattgttaaataccgatttaaaatttgagcttgataatttggctagatttatgcatatggcagtggattatgcgaaggaaatcgggtacacagggcagtttttgattgagccaaaaccaaaagagccgaccacccatcaatacgatacagatgcagcaacaaccattgcctttttgaagcaatatggcttagacaatcattttaaattaaatcttgaagccaatcatgccacattagccgggcatacattcgaacatgaattacgcatggcaagagtacatggtctgcttggctctgttgacgcaaaccagggtcatcctcttttaggctgggacacggatgaatttccgacggatttatattctacgacattagcaatgtacgaaatcctgcaaaatggcggccttggaagcggcggattaaactttgacgcgaaggtcagaagatcttctttcgagcctgatgatctaatatatgcccatattgcagggatggatgcatttgcaagaggattgaaagttgcccacaaattaatcgaagatcgtgtgtttgaagatgtgattcaacatcgttaccgcagctttactgaagggattggtcttgaaattatagaaggaagagctaatttccacacacttgagcaatatgcgctaaatcataaatcaattaaaaacgaatctggaagacaggagaaattaaaagcgatattgaaccaatacattttagaagta

SEQ ID NO: 35 - protein, 387 aa, Streptomyces rubiginosus
MNYQPTPEDRFTFGLWTVGWQGRDPFGDATRRALDPVESVRRLAELGAHGVTFHDDDLIPFGSSDSEREEHVKRFRQALDDTGMKVPMATTNLFTHPVFKDGGFTANDRDVRRYALRKTIRNIDLAVELGAETYVAWGGREGAESGGAKDVRDALDRMKEAFDLLGEYVTSQGYDIRFAIEPKPNEPRGDILLPTVGHALAFIERLERPELYGVNPEVGHEQMAGLNFPHGIAQALWAGKLFHIDLNGQNGIKYDQDLRFGAGDLRAAFWLVDLLESAGYSGPRHFDFKPPRTEDFDGVWASAAGCMRNYLILKERAAAFRADPEVQEALRASRLDELARPTAADGLQALLDDRSAFEEFDVDAAAARGMAFERLDQLAMDHLLGAR

SEQ ID NO: 36 - DNA, 1164 bp, Artificial sequence
  coding region codon optimized for expression in Saccharomyces cerevisiae
atgaactaccaaccaactccagaagatagattcactttcggtttgtggactgtcggttggcaaggtagagacccattcggtgacgctaccagaagagctttggacccagttgaatctgtcagaagattggctgaattgggtgctcacggtgttactttccacgacgatgacttgatcccattcggttcttccgactccgaaagagaagaacacgtcaagagattcagacaagctttggatgacaccggtatgaaggttccaatggctaccactaacttgttcacccacccagtcttcaaggacggtggtttcactgctaacgatagagacgttagaagatacgctttgagaaagaccatcagaaacatcgacttggctgttgaattgggtgctgaaacttacgtcgcttggggtggtagagaaggtgctgaatctggtggtgctaaggatgttagagacgctttggatagaatgaaggaagctttcgacttgttgggtgaatacgtcacctcccaaggttacgacatcagattcgctatcgaaccaaagccaaacgaaccaagaggtgacatcttgttgccaactgttggtcacgctttggctttcatcgaaagattggaaagaccagaattgtacggtgttaacccagaagtcggtcacgaacaaatggctggtttgaacttcccacacggtatcgctcaagctttgtgggctggtaaattgttccacatcgacttgaacggtcaaaacggtatcaagtacgatcaagacttgagattcggtgctggtgacttgagagctgctttctggttggttgatttgttggaatctgctggttactccggtccaagacacttcgacttcaagccaccaagaaccgaagatttcgacggtgtctgggcttctgctgctggttgtatgagaaactacttgatcttgaaggaaagagctgctgctttcagagctgacccagaagttcaagaagctttgagagcttctagattggacgaattggctagaccaactgctgctgatggtttgcaagctttgttggatgacagatccgctttcgaagaatttgacgttgacgctgctgctgctagaggtatggctttcgaaagattggaccaattggctatggatcacttgttgggtgctagaggt

SEQ ID NO: 37 - protein, 440 aa, Burkholderia phytofirmans
MSYFEHIPEIRYEGPQSDNPLAYRHYDKSKKVLGKTLEEHLRIAVCYWHTFVWPGVDIFGQGTFRRPWQQAGDAMERAQQKADSAFEFFSKLGTPYYTFHDTDVSPEGSNLKEYSENFLRITDYLARKQESTGIKLLWGTANLFSHPRYAAGAATSPDPEVFAFAATQVRHALDATQRLGGDNYVLWGGREGYDTLLNTDLVRERDQLARFLHMVVDHAHKIGFKGSLLIEPKPQEPTKHQYDYDVATVHGFLLQHGLDKEIRVNIEANHATLAGHSFHHEIATAYALGIFGSVDANRGDPQNGWDTDQFPNSVEELTLAFYEILKHGGFTTGGMNFDSKVRRQSVDPEDLFYGHIGAIDNLALAVERAAVLIENDRLDQFKRQRYSGWDAEFGRKISSGDYSLSALAEEAMARGLNPQHASGHQELMENIVNQAIYSGR

SEQ ID NO: 38 - DNA, 1320 bp, Artificial sequence
  coding region codon optimized for expression in Saccharomyces cerevisiae
atgtcctacttcgaacacatcccagaaatcagatacgaaggtccacaatccgataacccattggcttacagacactacgacaagtccaagaaggttttgggtaaaactttggaagaacacttgagaatcgctgtctgttactggcacactttcgtttggccaggtgttgacatcttcggtcaaggtactttcagaagaccatggcaacaagctggtgacgctatggaaagagcccaacaaaaggctgactctgctttcgaatttttctctaagttgggtactccatactacactttccacgacaccgatgtttctccagaaggttccaacttgaaggaatactctgaaaacttcttgagaatcactgactacttggctagaaagcaagaatccactggtatcaagttgttgtggggtactgctaacttgttctctcacccaagatacgctgctggtgctgctacctccccagacccagaagttttcgctttcgctgctactcaagtcagacacgctttggatgctacccaaagattgggtggtgacaactacgttttgtggggtggtagagaaggttacgacactttgttgaacaccgatttggtcagagaaagagaccaattggctagattcttgcacatggttgttgaccacgctcacaagatcggtttcaagggttctttgttgatcgaaccaaagccacaagaaccaactaagcaccaatacgactacgatgttgctaccgtccacggtttcttgttgcaacacggtttggacaaggaaatcagagtcaacatcgaagctaaccacgctactttggctggtcactctttccaccacgaaatcgctaccgcttacgctttgggtatcttcggttccgttgacgctaacagaggtgacccacaaaacggttgggacactgatcaattcccaaactctgtcgaagaattgaccttggctttctacgaaatcttgaagcacggtggtttcaccactggtggtatgaacttcgactctaaggttagaagacaatccgttgacccagaagatttgttctacggtcacatcggtgctatcgacaacttggctttggctgttgaaagagctgctgtcttgatcgaaaacgacagattggatcaattcaagagacaaagatactctggttgggatgctgaatttggtagaaagatctcttccggtgactactctttgtccgctttggctgaagaagctatggctagaggtttgaacccacaacacgcttctggtcaccaagaattgatggaaaacatcgttaaccaagctatctactccggtaga

SEQ ID NO: 39 - protein, 441 aa, Burkholderia phymatum
MSYFEHLPAVRYEGPQTDNPFAYRHYDKDKLVLGKRMEDHLRVAVCYWHTFVWPGADMFGPGTFERPWHHAGDALEMAHAKADHAFELFSKLGTPFYTFHDLDVAPEGDSIKSYVNNFKAMTDVLARKQEQTGIKLLWGTANLFSHPRYAAGAATNPNPDVFAFAATQVLNALEATQRLGGANYVLWGGREGYETLLNTDLKREREQLGRFMSMVVEHKHKTGFKGALLIEPKPQEPTKHQYDYDVATVHGFLTQFGLQDEIRVNIEANHATLAGHSFHHEIANAFALGIFGSVDANRGDAQNGWDTDQFPNSVEELTLAFYEILRNGGFTTGGMNFDAKVRRQSIDPEDIVHGHIGAIDVLAVALERAAHLIEHDRLAAFKQQRYAGWDSDFGRKILAGGYSLESLASDAVQRNIAPRHVSGQQERLENIVNQAIFSSAK

SEQ ID NO: 40 - DNA, 1323 bp, Artificial sequence
  coding region codon optimized for expression in Saccharomyces cerevisiae
atgtcctacttcgaacacttgccagctgtcagatacgaaggtccacaaaccgataacccattcgcttacagacactacgataaggataagttggttttgggtaaaagaatggaagaccacttgagagttgctgtctgttactggcacaccttcgtctggccaggtgctgacatgttcggtccaggtactttcgaaagaccatggcaccacgctggtgacgctttggaaatggctcacgctaaggctgatcacgctttcgaattgttctccaagttgggtactccattctacactttccacgacttggatgttgctccagaaggtgactctatcaagtcctacgttaacaacttcaaggctatgaccgatgtcttggctagaaagcaagaacaaaccggtatcaagttgttgtggggtactgctaacttgttctctcacccaagatacgctgctggtgctgctactaacccaaacccagacgttttcgctttcgctgctacccaagtcttgaacgctttggaagctactcaaagattgggtggtgctaactacgttttgtggggtggtagagaaggttacgaaaccttgttgaacactgacttgaagagagaaagagaacaattgggtagattcatgtctatggttgtcgaacacaagcacaagaccggtttcaagggtgctttgttgatcgaaccaaagccacaagaaccaactaagcaccaatacgactacgatgttgctaccgtccacggtttcttgactcaattcggtttgcaagacgaaatcagagtcaacatcgaagctaaccacgctaccttggctggtcactccttccaccacgaaatcgctaacgctttcgctttgggtatcttcggttctgttgacgctaacagaggtgacgctcaaaacggttgggacaccgatcaattcccaaactccgtcgaagaattgactttggctttctacgaaatcttgagaaacggtggtttcaccactggtggtatgaacttcgacgctaaggttagaagacaatctatcgacccagaagatatcgtccacggtcacatcggtgctatcgacgttttggctgtcgctttggaaagagctgctcacttgatcgaacacgatagattggctgctttcaagcaacaaagatacgctggttgggactccgatttcggtagaaagatcttggctggtggttactctttggaatccttggcttctgacgctgttcaaagaaacatcgctccaagacacgtctctggtcaacaagaaagattggaaaacatcgtcaaccaagctatcttctcttccgctaag

SEQ ID NO: 41 - protein, 444 aa, Citrobacter youngae
MELIMQAYFDQLDRVRFEGTKSTNPLAFRHYNPDEIVLGKRMEDHLRFAACYWHTFCWNGADMFGMGAFDRPWQQPGEALALAKRKADVAFEFFHKLNVPYYCFHDVDVSPEGASLKEYKNNFAQMVDVLAAKQEQSGVKLLWGTANCFTNPRYGAGAATNPDPEVFSWAATQVVTAMDATHKLGGENYVLWGGREGYETLLNTDLRQEREQIGRFMQLVVEHKHKIGFQGTLLIEPKPQEPTKHQYDYDAATVYGFLKQFGLEKEIKLNIEANHATLAGHSFHHEIATAIALGLFGSVDANRGDAQLGWDTDQFPNSVEENALVMYEILKAGGFTTGGLNFDAKVRRQSTDKYDLFYGHIGAMDTMALSLKIAARMIEDGGLDQRVAKRYAGWNGELGQQILKGQMTLTEIAQYAEQHNLAPVHQSGHQEQLENLVNHYLFDK

SEQ ID NO: 42 - DNA, 1332 bp, Artificial sequence
  coding region codon optimized for expression in Saccharomyces cerevisiae
atggaattgatcatgcaagcttacttcgaccaattggacagagtcagattcgaaggtactaagtctactaacccattggctttcagacactacaacccagacgaaatcgttttgggtaaaagaatggaagatcacttgagattcgctgcttgttactggcacaccttctgttggaacggtgctgacatgttcggtatgggtgctttcgatagaccatggcaacaaccaggtgaagctttggctttggctaagagaaaggctgacgttgctttcgaatttttccacaagttgaacgtcccatactactgtttccacgacgttgatgtctctccagaaggtgcttccttgaaggaatacaagaacaacttcgctcaaatggttgacgttttggctgctaagcaagaacaatctggtgtcaagttgttgtggggtactgctaactgtttcactaacccaagatacggtgctggtgctgctaccaacccagacccagaagttttctcctgggctgctacccaagttgtcactgctatggatgctactcacaagttgggtggtgaaaactacgtcttgtggggtggtagagaaggttacgaaaccttgttgaacactgacttgagacaagaaagagaacaaatcggtagattcatgcaattggttgtcgaacacaagcacaagatcggtttccaaggtactttgttgatcgaaccaaagccacaagaaccaaccaagcaccaatacgactacgatgctgctactgtttacggtttcttgaagcaattcggtttggaaaaggaaatcaagttgaacatcgaagctaaccacgctaccttggctggtcactctttccaccacgaaatcgctactgctatcgctttgggtttgttcggttccgttgacgctaacagaggtgacgctcaattgggttgggacactgatcaattcccaaactctgttgaagaaaacgctttggtcatgtacgaaatcttgaaggctggtggtttcaccactggtggtttgaacttcgacgctaaggttagaagacaatctaccgacaagtacgatttgttctacggtcacatcggtgctatggacactatggctttgtccttgaagatcgctgctagaatgatcgaagacggtggtttggatcaaagagtcgctaagagatacgctggttggaacggtgaattgggtcaacaaatcttgaagggtcaaatgaccttgactgaaatcgctcaatacgctgaacaacacaacttggctccagttcaccaatctggtcaccaagaacaattggaaaacttggtcaaccactacttgttcgacaag

SEQ ID NO: 43 - protein, 440 aa, Escherichia blattae
MPTYFDQIDRVRFEGPKTTNPLAFRHYNPDELVLGKRMEDHLRFAACYWHNFCWNGADMFGVGSFDRPWQHPGSALEMARQKADVAFEFFHKLNVPYYCFHDVDVSPEGASLKEYLENFAHMVDVLAEKQQQSGVKLLWGTANCFTNPRFGAGAATNPDPEVFAMAATQVFTAMNATQKLGGENYVLWGGREGYESLLNTDLRQEREQIGRFMQMVVEHKHKIGFRGTLLIEPKPQEPTKHQYDYDVATVYGFLKQFGLEKEIKVNIEANHATLAGHSFHHEIASAIALGIFGSVDANRGDAQLGWDTDQFPNSVEENSLVMYEILKAGGFTTGGLNFDAKVRRQSTDKYDLFYGHIGAMDTMALSLKIAARMIEDGELDKRVARRYSGWSSELGQQILKGQMSLAQLAQYAQQHQLDPHHQSGHQELLENLVNHYIFDK

SEQ ID NO: 44 - DNA, 1320 bp, Artificial sequence
  coding region codon optimized for expression in Saccharomyces cerevisiae
atgccaacttacttcgatcaaatcgacagagtcagattcgaaggtccaaagaccactaacccattggctttcagacactacaacccagacgaattggttttgggtaaaagaatggaagatcacttgagattcgctgcttgttactggcacaacttctgttggaacggtgctgacatgttcggtgtcggttctttcgatagaccatggcaacacccaggttccgctttggaaatggctagacaaaaggctgacgttgctttcgaatttttccacaagttgaacgtcccatactactgtttccacgacgttgatgtctctccagaaggtgcttccttgaaggaatacttggaaaacttcgctcacatggttgacgttttggctgaaaagcaacaacaatctggtgttaagttgttgtggggtactgctaactgtttcactaacccaagattcggtgctggtgctgctaccaacccagacccagaagttttcgctatggctgctacccaagtcttcactgctatgaacgctactcaaaagttgggtggtgaaaactacgtcttgtggggtggtagagaaggttacgaatctttgttgaacaccgacttgagacaagaaagagaacaaatcggtagattcatgcaaatggttgtcgaacacaagcacaagatcggtttcagaggtactttgttgatcgaaccaaagccacaagaaccaaccaagcaccaatacgactacgatgttgctactgtctacggtttcttgaagcaattcggtttggaaaaggaaatcaaggttaacatcgaagctaaccacgctaccttggctggtcactctttccaccacgaaatcgcttccgctatcgctttgggtatcttcggttctgttgacgctaacagaggtgacgctcaattgggttgggacactgatcaattcccaaactctgttgaagaaaactccttggtcatgtacgaaatcttgaaggctggtggtttcaccactggtggtttgaacttcgacgctaaggttagaagacaatctaccgacaagtacgatttgttctacggtcacatcggtgctatggacactatggctttgtccttgaagatcgctgctagaatgatcgaagacggtgaattggataagagagtcgctagaagatactctggttggtcttccgaattgggtcaacaaatcttgaagggtcaaatgtccttggctcaattggctcaatacgctcaacaacaccaattggacccacaccaccaatctggtcaccaagaattgttggaaaacttggttaaccactacatcttcgataag

SEQ ID NO: 45 - protein, 438 aa, Pseudomonas fluorescens
MPYFPGVEKVRFEGPASTSALAFRHYDANKLILGKPMREHLRMAACYWHTFVWPGADMFGMGTFKRPWQRSGDPMEVAIGKAEAAFEFFSKLGIDYYSFHDTDVAPEGSSLKEYRNHFAQMVDHLERHQEQTGIKLLWGTANCFSNPRFAAGAASNPDPEVFAFAAAQVFSAMNATLRLKGANYVLWGGREGYETLLNTDLKREREQLGRFMRMVVEHKHKIGFKGDLLIEPKPQEPTKHQYDYDSATVFGFLHEYGLEHEIKVNIEANHATLAGHSFHHEIATAVSLGIFGSIDANRGDPQNGWDTDQFPNSVEEMTLATYEILKAGGFKNGGYNFDSKVRRQSLDEVDLFHGHVAAMDVLALALERAAAMVQDDRLQQFKEQRYAGWQQPLGQAVLAGEFSLESLAEHAFANELNPQAVSGRQEMLEGVVNRFIYR

SEQ ID NO: 46 - DNA, 1314 bp, Artificial sequence
  coding region codon optimized for expression in Saccharomyces cerevisiae
atgccatacttcccaggtgttgaaaaggtcagattcgaaggtccagcttccacttccgctttggctttcagacactacgacgctaacaagttgatcttgggtaaaccaatgagagaacacttgagaatggctgcttgttactggcacaccttcgtctggccaggtgctgacatgttcggtatgggtactttcaagagaccatggcaaagatctggtgacccaatggaagttgctatcggtaaagctgaagctgctttcgaatttttctctaagttgggtatcgactactactccttccacgacaccgatgttgctccagaaggttcttccttgaaggaatacagaaaccacttcgctcaaatggttgaccacttggaaagacaccaagaacaaaccggtatcaagttgttgtggggtactgctaactgtttctctaacccaagattcgctgctggtgctgcttccaacccagacccagaagttttcgctttcgctgctgctcaagtcttctctgctatgaacgctactttgagattgaagggtgctaactacgtcttgtggggtggtagagaaggttacgaaaccttgttgaacactgacttgaagagagaaagagaacaattgggtagattcatgagaatggttgtcgaacacaagcacaagatcggtttcaagggtgacttgttgatcgaaccaaagccacaagaaccaaccaagcaccaatacgactacgattctgctactgttttcggtttcttgcacgaatacggtttggaacacgaaatcaaggtcaacatcgaagctaaccacgctaccttggctggtcactccttccaccacgaaatcgctactgctgtctctttgggtatcttcggttccatcgatgctaacagaggtgacccacaaaacggttgggacaccgatcaattcccaaactctgttgaagaaatgaccttggctacttacgaaatcttgaaggctggtggtttcaagaacggtggttacaacttcgactctaaggttagaagacaatccttggacgaagtcgatttgttccacggtcacgttgctgctatggatgtcttggctttggctttggaaagagctgctgctatggttcaagacgatagattgcaacaattcaaggaacaaagatacgctggttggcaacaaccattgggtcaagctgtcttggctggtgaattttctttggaatccttggctgaacacgctttcgctaacgaattgaacccacaagctgtttctggtagacaagaaatgttggaaggtgttgtcaacagattcatctacaga

SEQ ID NO: 47 - protein, 439 aa, Photobacterium profundum
MTEFFKNINKIQFEGTDAINPLAFRHYDAERMILGKSMKEHLRFAACYWHNFCWPGSDVFGAATFDRPWLQSGNAMEMAHMKADAAFDFFSKLGVPYYCFHDTDIAPEGTSLKEYVNNFAQMVDVLEQKQDETGLKLLWGTANAFSNPRYMSGAGTNPDPKVFAYAATQIFNAMGATQRLGGENYVLWGGREGYETLLNTDLRQEREQLGRLMQMVVEHKHKIGFKGTILIEPKPQEPTKHQYDYDTATVYGFLKQFGLENEIKVNIEANHATLAGHSFQHEIATATSLGLFGSIDANRGDPQLGWDTDQFPNSVEENTLVMYEILKAGGFTTGGFNFDSHVRRPSIDAEDLFYGHIGGMDTMALALERAANMIENDVLSKNIAQRYAGWNEDLGKKILSGDHSLETLAKFALDSNIAPVKESGRQEHLENIVNGFIYK

SEQ ID NO: 48 - DNA, 1317 bp, Artificial sequence
  coding region codon optimized for expression in Saccharomyces cerevisiae
atgaccgagttcttcaagaacatcaacaagatccaattcgaaggtactgacgctatcaacccattggctttcagacactacgacgctgaaagaatgatcttgggtaaatctatgaaggaacacttgagattcgctgcttgttactggcacaacttctgttggccaggttctgacgttttcggtgctgctaccttcgatagaccatggttgcaatccggtaacgctatggaaatggctcacatgaaggctgacgctgctttcgatttcttctctaagttgggtgttccatactactgtttccacgacaccgatatcgctccagaaggtacttccttgaaggaatacgtcaacaacttcgctcaaatggttgacgttttggaacaaaagcaagatgaaaccggtttgaagttgttgtggggtactgctaacgctttctctaacccaagatacatgtccggtgctggtactaacccagacccaaaggttttcgcttacgctgctacccaaatcttcaacgctatgggtgctactcaaagattgggtggtgaaaactacgtcttgtggggtggtagagaaggttacgaaaccttgttgaacactgacttgagacaagaaagagaacaattgggtagattgatgcaaatggttgtcgaacacaagcacaagatcggtttcaagggtactatcttgatcgaaccaaagccacaagaaccaactaagcaccaatacgactacgataccgctactgtttacggtttcttgaagcaattcggtttggaaaacgaaatcaaggtcaacatcgaagctaaccacgctaccttggctggtcactctttccaacacgaaatcgctaccgctacttctttgggtttgttcggttccatcgatgctaacagaggtgacccacaattgggttgggacaccgatcaattcccaaactctgttgaagaaaacactttggtcatgtacgaaatcttgaaggctggtggtttcaccactggtggtttcaacttcgactctcacgttagaagaccatccatcgacgctgaagatttgttctacggtcacatcggtggtatggacaccatggctttggctttggaaagagctgctaacatgatcgaaaacgacgttttgtctaagaacatcgctcaaagatacgctggttggaacgaagacttgggtaaaaagatcttgtctggtgaccactccttggaaactttggctaagttcgctttggactccaacatcgctccagttaaggaatctggtagacaagaacacttggaaaacatcgtcaacggtttcatctacaag

SEQ ID NO: 49 - protein, 440 aa, Pantoea stewartii
MHAYFDQLDRVRYEGAKTINPLAFRHYNPDEVILGKTMAEHLRFAACYWHTFCWNGADMFGVGAFDRPWQKAGDALALAKLKADVAFEFFHKLNVPYYCFHDVDVSPEGDSLKSYRENLAVMTDTLQAKQQETGLKLLWGTANCFTHPRYGAGAATNPDPEVFSWAASQVCSAMKATQTLGGENYVLWGGREGYETLLNTDLRQEREQIGRFMQMVVEHKHKIGFQGTLLIEPKPQEPTKHQYDYDVATVYGFLKQFGLEKEIKVNVEANHATLAGHSFHHEIATAIALGVFGSVDANRGDAQCGWDTDQFPVSVEENALVMYEIIKAGGFTTGGLNFDAKVRRQSTDKYDLFYGHIGAMDTMALALKVAARMLSDGELDQRVAQRYSGWNGEFGQQILKGEFSLETLAAHAHQQQFNPQHRSGRQEQLENLVNHYLYDF

SEQ ID NO: 50 - DNA, 1320 bp, Artificial sequence
  coding region codon optimized for expression in Saccharomyces cerevisiae
atgcacgcttacttcgatcaattggacagagtcagatacgaaggtgctaagaccatcaacccattggctttcagacactacaacccagacgaagttatcttgggtaaaaccatggctgaacacttgagattcgctgcttgttactggcacactttctgttggaacggtgctgacatgttcggtgtcggtgctttcgatagaccatggcaaaaggctggtgacgctttggctttggctaagttgaaggctgacgttgctttcgaatttttccacaagttgaacgtcccatactactgtttccacgacgttgatgtctctccagaaggtgactctttgaagtcctacagagaaaacttggctgttatgaccgacactttgcaagctaagcaacaagaaaccggtttgaagttgttgtggggtactgctaactgtttcactcacccaagatacggtgctggtgctgctactaacccagacccagaagttttctcttgggctgcttcccaagtctgttctgctatgaaggctacccaaactttgggtggtgaaaactacgtcttgtggggtggtagagaaggttacgaaaccttgttgaacactgacttgagacaagaaagagaacaaatcggtagattcatgcaaatggttgtcgaacacaagcacaagatcggtttccaaggtactttgttgatcgaaccaaagccacaagaaccaaccaagcaccaatacgactacgatgttgctactgtctacggtttcttgaagcaattcggtttggaaaaggaaatcaaggttaacgtcgaagctaaccacgctaccttggctggtcactccttccaccacgaaatcgctactgctatcgctttgggtgttttcggttctgttgacgctaacagaggtgacgctcaatgtggttgggacactgatcaattcccagtttccgtcgaagaaaacgctttggttatgtacgaaatcatcaaggctggtggtttcaccactggtggtttgaacttcgatgctaaggtcagaagacaatctaccgacaagtacgatttgttctacggtcacatcggtgctatggacactatggctttggctttgaaggttgctgctagaatgttgtccgacggtgaattggatcaaagagtcgctcaaagatactctggttggaacggtgaatttggtcaacaaatcttgaagggtgaattttctttggaaaccttggctgctcacgctcaccaacaacaattcaacccacaacacagatctggtagacaagaacaattggaaaacttggttaaccactacttgtacgacttc

SEQ ID NO: 51 - protein, 440 aa, Plautia stali symbiont
MHAYFDQLERVGYEGANTTNALAFRHYNPQEVILGKTMAEHLRFAACYWHTFCWNGADMFGVGAFDRPWQKNGDALQLAKLKADVAFEFFYKLNVPYYCFHDVDVSPEGDSLRSYQENLAVITDKLLEKQQETGVKLLWGTANCFTHPRYAAGAATSPDPEIFAWAASQVCSAMQATQTLGGENYVLWGGREGYETLLNTDLRQEREQIGRFMQMVVEHKHKIGFQGMLLIEPKPQEPTKHQYDFDVAMVYGFLRQFGLEKEIKVNVEANHATLAGHSFHHEIATAIALGIFGSVDANRGDSQCGWDTDQFPNSVEENALVMYEILKAGGFTTGGLNFDAKVRRQSTDKYDLFYGHIGAMDTMALALKVAARMVSDGELDKRVAQRYSGWNGEFGQQILKGEFSLASLAAHAQQLQLNPQHRSGRQEQLENLVNHYLYNF

SEQ ID NO: 52 - DNA, 1320 bp, Artificial sequence
  Artificial sequence
atgcacgcttacttcgatcaattggaaagagtcggttacgaaggtgctaacactactaacgctttggctttcagacactacaacccacaagaagttatcttgggtaaaaccatggctgaacacttgagattcgctgcttgttactggcacactttctgttggaacggtgctgacatgttcggtgtcggtgctttcgatagaccatggcaaaagaacggtgacgctttgcaattggctaagttgaaggctgacgttgctttcgaatttttctacaagttgaacgtcccatactactgtttccacgacgttgatgtctctccagaaggtgactctttgagatcctaccaagaaaacttggctgttatcaccgacaagttgttggaaaagcaacaagaaactggtgtcaagttgttgtggggtactgctaactgtttcactcacccaagatacgctgctggtgctgctacctccccagacccagaaatcttcgcttgggctgcttctcaagtttgttccgctatgcaagctacccaaactttgggtggtgaaaactacgtcttgtggggtggtagagaaggttacgaaaccttgttgaacactgacttgagacaagaaagagaacaaatcggtagattcatgcaaatggttgtcgaacacaagcacaagatcggtttccaaggtatgttgttgatcgaaccaaagccacaagaaccaaccaagcaccaatacgacttcgatgttgctatggtctacggtttcttgagacaattcggtttggaaaaggaaatcaaggttaacgtcgaagctaaccacgctaccttggctggtcactctttccaccacgaaatcgctactgctatcgctttgggtatcttcggttctgttgacgctaacagaggtgactcccaatgtggttgggacactgatcaattcccaaactctgttgaagaaaacgctttggtcatgtacgaaatcttgaaggctggtggtttcaccactggtggtttgaacttcgacgctaaggttagaagacaatccaccgacaagtacgatttgttctacggtcacatcggtgctatggacactatggctttggctttgaaggttgctgctagaatggtctctgacggtgaattggataagagagtcgctcaaagatactccggttggaacggtgaatttggtcaacaaatcttgaagggtgaattttctttggcttctttggctgctcacgctcaacaattgcaattgaacccacaacacagatctggtagacaagaacaattggaaaacttggtcaaccactacttatacaacttc

SEQ ID NO: 53 - protein, 438 aa, Pseudomonas syringae
MSYFPTVDKVIYEGPDSDSPLAFRHYDADRRVLGKPMREHLRMAACYWHSFVWPGADMFGVGTFKRPWQRAGDPMELAIGKAEAAFEFFSKLGIDYYSFHDTDVAPEGSSIREYQNNFAQMVDRLERHQEQSGIKLLWGTANCFSNPRFAAGAASNPDPEVFAYAGAQVFSAMNATQRLKGSNYVLWGGREGYETLLNTDLKREREQLGRFMRMVVEHKHKIGFKGDLLIEPKPQEPTKHQYDYDSATVFGFLHQYGLQDEIKVNIEANHATLAGHSFHHEIATAVSLGIFGSIDANRGDPQNGWDTDQFPNSVEEMTLATYEILKAGGFTHGGYNFDSKVRRQSLDDVDLFHGHVAAMDVLALSLERAAAMVQNDKLQQFKDQRYAGWQQPFGQSVLSGGFSLASLAEHAFANELNPQAVSGRQELLEGVVNRFIYT

SEQ ID NO: 54 - DNA, 1314 bp, Artificial sequence
  coding region codon optimized for expression in Saccharomyces cerevisiae
atgtcctacttcccaaccgttgataaggtcatctacgaaggtccagactccgactccccattggctttcagacactacgacgctgatagaagagtcttgggtaaaccaatgagagaacacttgagaatggctgcttgttactggcactctttcgtttggccaggtgctgacatgttcggtgtcggtactttcaagagaccatggcaaagagctggtgacccaatggaattggctatcggtaaagctgaagctgctttcgaatttttctctaagttgggtatcgactactactccttccacgacactgatgttgctccagaaggttcttccatcagagaataccaaaacaacttcgctcaaatggttgacagattggaaagacaccaagaacaatctggtatcaagttgttgtggggtactgctaactgtttctctaacccaagattcgctgctggtgctgcttccaacccagacccagaagttttcgcttacgctggtgctcaagtcttctctgctatgaacgctactcaaagattgaagggttccaactacgttttgtggggtggtagagaaggttacgaaaccttgttgaacactgacttgaagagagaaagagaacaattgggtagattcatgagaatggttgtcgaacacaagcacaagatcggtttcaagggtgacttgttgatcgaaccaaagccacaagaaccaaccaagcaccaatacgactacgattctgctactgttttcggtttcttgcaccaatacggtttgcaagacgaaatcaaggtcaacatcgaagctaaccacgctaccttggctggtcactccttccaccacgaaatcgctactgctgtctctttgggtatcttcggttccatcgatgctaacagaggtgacccacaaaacggttgggacaccgatcaattcccaaactctgttgaagaaatgaccttggctacttacgaaatcttgaaggctggtggtttcactcacggtggttacaacttcgactctaaggttagaagacaatccttggacgacgttgacttgttccacggtcacgttgctgctatggatgtcttggctttgtctttggaaagagctgctgctatggttcaaaacgacaagttgcaacaattcaaggatcaaagatacgctggttggcaacaaccattcggtcaatctgtcttgtccggtggtttctctttggcttccttggctgaacacgctttcgctaacgaattgaacccacaagctgtttctggtagacaagaattgttggaaggtgttgtcaacagattcatctacacc

SEQ ID NO: 55 - protein, 439 aa, Vibrio sp.
MTEFFKNINKINFEGAESTNPLAFRHYDADKMILGKSMAEHLRFAACYWHNFRWGGADIFGDGTFEHAWLNAADPMEQALMKADAAFEFFTKLGVPYYCFHDTDVAPEGNSIKEYINNFQTMVDVLEQKQEETGMKLLWGTANAFSNARYMAGAGTNPDPKVFAYAATQIFNAMGATQRLGGENYVLWGGREGYETLLNTDLRQEREQLGRLMQMVVEHKHKIGFKGSILIEPKPQEPTKHQYDYDTATVYGFLKQFGLENEIKVNIEANHATLAGHSFHHEVATATSLGLFGSIDANRGDPQLGWDTDQFPNSVEENTLVMYEILKAGGFTTGGFNFDARVRRPSTELEDLFHGHIGGMDTMALSLERAANMIENDVLSKNIAERYAGWNDDLGQKILKGDLSLAGLAAFTEETNINPVKESGRQEYLENVVNGFIYK

SEQ ID NO: 56 - DNA, 1317 bp, Artificial sequence
  coding region codon optimized for expression in Saccharomyces cerevisiae
atgaccgagttcttcaagaacatcaacaagatcaacttcgaaggtgctgaatccactaacccattggctttcagacactacgacgctgacaagatgatcttgggtaaatctatggctgaacacttgagattcgctgcttgttactggcacaacttcagatggggtggtgctgacatcttcggtgacggtactttcgaacacgcttggttgaacgctgctgacccaatggaacaagctttgatgaaggctgatgctgctttcgaatttttcaccaagttgggtgttccatactactgtttccacgacactgatgtcgctccagaaggtaactctatcaaggaatacatcaacaacttccaaaccatggttgacgttttggaacaaaagcaagaagaaaccggtatgaagttgttgtggggtactgctaacgctttctccaacgctagatacatggctggtgctggtactaacccagacccaaaggttttcgcttacgctgctacccaaatcttcaacgctatgggtgctactcaaagattgggtggtgaaaactacgtcttgtggggtggtagagaaggttacgaaaccttgttgaacactgacttgagacaagaaagagaacaattgggtagattgatgcaaatggttgtcgaacacaagcacaagatcggtttcaagggttctatcttgatcgaaccaaagccacaagaaccaaccaagcaccaatacgactacgataccgctactgtttacggtttcttgaagcaattcggtttggaaaacgaaatcaaggtcaacatcgaagctaaccacgctactttggctggtcactccttccaccacgaagttgctaccgctacttctttgggtttgttcggttccatcgacgctaacagaggtgacccacaattgggttgggacaccgatcaattcccaaactctgttgaagaaaacactttggtcatgtacgaaatcttgaaggctggtggtttcaccactggtggtttcaacttcgacgctagagttagaagaccatccaccgaattggaagacttgttccacggtcacatcggtggtatggatactatggctttgtctttggaaagagctgctaacatgatcgaaaacgacgttttgtccaagaacatcgctgaaagatacgctggttggaacgacgatttgggtcaaaagatcttgaagggtgacttgtctttggctggtttggctgctttcaccgaagaaactaacatcaacccagttaaggaatctggtagacaagaatacttggaaaacgtcgtcaacggtttcatctacaag

SEQ ID NO: 57 - protein, 444 aa, Yokenella regensburgei
MEFIMQSYFDQLERVRYEGPKSENPLAFRHYNPDELVLGKRMEEHLRFAACYWHTFCWNGADMFGVGAFERPWQQAGDALALAKRKADVAFEFFHKLNVPYYCFHDVDVSPEGASLKEYRNNFAQMVDVLAQKQQESGVKLLWGTANCFTNPRYGAGAATNPDPEVFSWAATQVVTAMDATHRLGGENYVLWGGREGYETLLNTDLRQEREQIGRFMQMVVEHKHKTGFQGTLLIEPKPQEPTKHQYDYDAATVYGFLKQFGLEKEIKLNIEANHATLAGHSFHHEIATAIALGLFGSVDANRGDAQLGWDTDQFPNSVEENALVMYEILKAGGFTTGGLNFDAKVRRQSTDKYDLFYGHIGAMDTMALALKVAARMVEDGQLDKRVAKRYAGWNGELGQQILKGQMSLTELATYAEQHNLAPQHHSGHQELLENLVNHYLFDK

SEQ ID NO: 58 - DNA, 1332 bp, Artificial sequence
  coding region codon optimized for expression in Saccharomyces cerevisiae
atggagttcatcatgcaatcctacttcgatcaattggaaagagttagatacgaaggtccaaagtccgaaaacccattggctttcagacactacaacccagacgaattggttttgggtaaaagaatggaagaacacttgagattcgctgcttgttactggcacaccttctgttggaacggtgctgacatgttcggtgtcggtgctttcgaaagaccatggcaacaagctggtgacgctttggctttggctaagagaaaggctgatgttgctttcgaatttttccacaagttgaacgtcccatactactgtttccacgacgttgatgtctctccagaaggtgcttccttgaaggaatacagaaacaacttcgctcaaatggttgacgttttggctcaaaagcaacaagaatctggtgttaagttgttgtggggtactgctaactgtttcactaacccaagatacggtgctggtgctgctaccaacccagacccagaagttttctcctgggctgctacccaagttgtcactgctatggatgctactcacagattgggtggtgaaaactacgtcttgtggggtggtagagaaggttacgaaaccttgttgaacactgacttgagacaagaaagagaacaaatcggtagattcatgcaaatggttgtcgaacacaagcacaagaccggtttccaaggtactttgttgatcgaaccaaagccacaagaaccaaccaagcaccaatacgactacgatgctgctactgtttacggtttcttgaagcaattcggtttggaaaaggaaatcaagttgaacatcgaagctaaccacgctaccttggctggtcactctttccaccacgaaatcgctactgctatcgctttgggtttgttcggttccgttgacgctaacagaggtgacgctcaattgggttgggacactgatcaattcccaaactctgttgaagaaaacgctttggtcatgtacgaaatcttgaaggctggtggtttcaccactggtggtttgaacttcgacgctaaggttagaagacaatccaccgacaagtacgatttgttctacggtcacatcggtgctatggacactatggctttggctttgaaggttgctgctagaatggtcgaagacggtcaattggataagagagtcgctaagagatacgctggttggaacggtgaattgggtcaacaaatcttgaagggtcaaatgtctttgaccgaattggctacttacgctgaacaacacaacttggctccacaacaccactccggtcaccaagaattgttggaaaacttggtcaaccactacttgttcgataag

SEQ ID NO: 59 - DNA, 1182 bp, Artificial sequence
  coding region codon optimized for expression in Saccharomyces cerevisiae
atgtccgttcaagctaccagagaagacaagttctccttcggtttgtggactgtcggttggcaagctagagacgctttcggtgacgctaccagaactgctttggacccagttgaagctgtccacaagttggctgaaatcggtgcttacggtatcaccttccacgacgatgacttggttccattcggttctgacgctcaaactagagatggtatcatcgctggtttcaagaaggctttggacgaaaccggtttgatcgttccaatggtcaccactaacttgttcacccacccagtcttcaaggatggtggtttcacttctaacgacagatccgttagaagatacgctatcagaaaggtcttgagacaaatggacttgggtgctgaattgggtgctaagactttggttttgtggggtggtagagaaggtgctgaatacgactctgctaaggatgtctccgctgctttggatagatacagagaagctttgaacttgttggctcaatactctgaagacagaggttacggtttgagattcgctatcgaaccaaagccaaacgaaccaagaggtgacatcttgttgccaaccgctggtcacgctatcgctttcgttcaagaattggaaagaccagaattgttcggtatcaacccagaaaccggtcacgaacaaatgtctaacttgaacttcactcaaggtatcgctcaagctttgtggcacaagaagttgttccacatcgacttgaacggtcaacacggtccaaagttcgatcaagacttggttttcggtcacggtgacttgttgaacgctttctctttggttgacttgttggaaaacggtccagacggtgctccagcttacgatggtccaagacacttcgactacaagccatctagaactgaagattacgacggtgtctgggaatccgctaaggctaacatcagaatgtacttgttgttgaaggaaagagctaaggctttcagagctgacccagaagttcaagaagctttggctgcttctaaggtcgctgaattgaagaccccaactttgaacccaggtgaaggttacgctgaattgttggctgacagatccgctttcgaagattacgacgctgatgctgttggtgctaagggtttcggtttcgttaagttgaaccaattggctatcgaacacttgttgggtgctaga

SEQ ID NO: 60 - DNA, 1320 bp, Artificial sequence; identical to SEQ ID NO: 32
  coding region codon optimized for expression in Saccharomyces cerevisiae

SEQ ID NO: 61 - DNA, 3036 bp, Artificial sequence
  chimeric AMxylA expression cassette  ILV5p-Am XI coding-ILV5t with a 5′ NotI site and a 3′ PmeI site
gcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatgtccgttcaagccacaagagaagacaagtttagtttcggtttatggactgtaggttggcaagcaagagacgcattcggtgacgcaaccagaactgccttggatccagttgaagctgtccataaattggcagaaatcggtgcctacggtattacattccacgatgacgatttggttccttttggttccgatgctcaaaccagagacggtattatagccggtttcaaaaaggctttagatgaaactggtttgatcgtaccaatggttactacaaatttgtttactcatcctgtcttcaaggacggtggttttacatctaacgatagatcagtcagaagatacgctataagaaaggtattgagacaaatggatttgggtgctgaattgggtgcaaagacattagtcttgtggggtggtagagaaggtgcagaatacgattccgccaaagacgttagtgctgcattggacagatatagagaagcattgaatttgttggcacaatactctgaagatagaggttacggtttgagatttgctatagaaccaaagcctaacgaaccaagaggtgacatattgttacctactgcaggtcatgcaatcgccttcgttcaagaattggaaagaccagaattgttcggtattaatcctgaaaccggtcacgaacaaatgtctaatttgaacttcactcaaggtattgctcaagcattatggcataaaaagttgttccacatcgatttgaacggtcaacatggtccaaaattcgaccaagatttggtatttggtcacggtgacttgttgaacgctttctcattggttgatttgttggaaaacggtccagatggtgcccctgcttatgacggtccaagacattttgattacaaaccttctagaacagaagactatgatggtgtttgggaatcagcaaaggccaacatcagaatgtacttgttgttgaaggaaagagctaaggcattcagagcagatccagaagttcaagaagccttagccgcttccaaagtcgcagaattgaagacaccaaccttaaatcctggtgaaggttacgccgaattattggctgatagaagtgcatttgaagactatgatgccgacgctgttggtgctaaaggttttggttttgtcaagttaaatcaattagcaatcgaacacttattaggtgccagatgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgag

SEQ ID NO: 62 - DNA, 1247 bp, Artificial sequence
  GPDp-ECgroES-CYC1t  with a  5′ PacI site and a 3′ NotI site
agatctagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaatattagaccattgcatgatagagttattgttaagagaaaggaagttgaaaccaaatctgcaggtggtattgttttgactggttccgctgcagctaagagtacaagaggtgaagttttggctgttggtaatggtagaattttagaaaacggtgaagttaagcctttggatgttaaggttggtgacattgttattttcaatgatggttacggtgttaagtcagaaaagattgataacgaagaagttttgatcatgtctgaatcagatatcttggcaattgttgaagcataattaattaatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcgggcggccgc

SEQ ID NO: 63 - DNA, 2678 bp, Artificial sequence
  ADH1p-ECgroEL-ADH1t with a 5′ PacI site and a 3′ SpeI site
gaattcctgcagcccgggggatccttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatggctgctaaagatgtaaagttcggtaatgatgctagagtaaaaatgttgagaggtgtaaatgtattggctgacgctgtaaaagtaactttgggtccaaaaggtagaaatgttgtcttggataagtcttttggtgctcctaccataactaaagacggtgtttcagtcgcaagagaaatcgaattggaggataagttcgaaaacatgggtgctcaaatggtcaaagaagtcgcctctaaggctaacgatgctgcaggtgacggtactacaaccgctactgttttggctcaagcaattataacagaaggtttaaaagcagttgccgctggtatgaatccaatggatttgaaaagaggtattgacaaggccgtcactgcagccgtagaagaattgaaagcattatcagtcccttgttctgattcaaaggccatcgctcaagtaggtaccatttccgctaacagtgatgaaactgttggtaaattaattgcagaagccatggacaaagtcggtaaagaaggtgtaataaccgttgaagatggtactggtttgcaagatgaattagacgtagttgagggtatgcaatttgatagaggttatttgtcaccatacttcatcaataagcctgaaacaggtgctgttgaattggaatccccttttattttgttggcagataaaaagattagtaacataagagaaatgttgccagttttagaagctgtcgcaaaagccggtaaacctttgttaatcattgctgaagatgttgaaggtgaagcattggcaacattagtcgtaaataccatgagaggtattgtaaaagttgctgcagttaaggctccaggtttcggtgacagaagaaaagctatgttgcaagacattgcaacattaaccggtggtacagttatctccgaagaaattggtatggaattggaaaaggccaccttggaagatttgggtcaagctaagagagttgtcattaataaggatactacaaccatcatcgacggtgtaggtgaagaagccgctatacaaggtagagttgctcaaataagacaacaaatcgaagaagcaacttctgattatgacagagaaaaattgcaagaaagagttgcaaagttagccggtggtgtcgctgtaattaaagttggtgcagccaccgaagtcgaaatgaaggaaaagaaagcaagagtagaagatgctttgcatgcaacaagagctgcagttgaagaaggtgtagttgcaggtggtggtgtcgccttaattagagtagcctccaaattggctgatttgagaggtcaaaatgaagaccaaaacgtaggtatcaaggttgccttaagagctatggaagcaccattgagacaaatcgttttgaactgtggtgaagaacctagtgtcgtagctaacactgttaaaggtggtgacggtaattatggttacaacgccgctacagaagaatacggtaacatgatcgatatgggtatattggacccaactaaggtcacaagatctgcattgcaatacgcagcctcagttgccggtttaatgattactacagaatgcatggttacagatttgcctaaaaacgacgctgccgacttgggtgccgcaggtggtatgggtggtatgggtggtatgggtggtatgatgtgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtactagt

SEQ ID NO: 64 - DNA, 9766 bp, Artificial sequence
  constructed plasmid
ggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatgtccgttcaagccacaagagaagacaagtttagtttcggtttatggactgtaggttggcaagcaagagacgcattcggtgacgcaaccagaactgccttggatccagttgaagctgtccataaattggcagaaatcggtgcctacggtattacattccacgatgacgatttggttccttttggttccgatgctcaaaccagagacggtattatagccggtttcaaaaaggctttagatgaaactggtttgatcgtaccaatggttactacaaatttgtttactcatcctgtcttcaaggacggtggttttacatctaacgatagatcagtcagaagatacgctataagaaaggtattgagacaaatggatttgggtgctgaattgggtgcaaagacattagtcttgtggggtggtagagaaggtgcagaatacgattccgccaaagacgttagtgctgcattggacagatatagagaagcattgaatttgttggcacaatactctgaagatagaggttacggtttgagatttgctatagaaccaaagcctaacgaaccaagaggtgacatattgttacctactgcaggtcatgcaatcgccttcgttcaagaattggaaagaccagaattgttcggtattaatcctgaaaccggtcacgaacaaatgtctaatttgaacttcactcaaggtattgctcaagcattatggcataaaaagttgttccacatcgatttgaacggtcaacatggtccaaaattcgaccaagatttggtatttggtcacggtgacttgttgaacgctttctcattggttgatttgttggaaaacggtccagatggtgcccctgcttatgacggtccaagacattttgattacaaaccttctagaacagaagactatgatggtgtttgggaatcagcaaaggccaacatcagaatgtacttgttgttgaaggaaagagctaaggcattcagagcagatccagaagttcaagaagccttagccgcttccaaagtcgcagaattgaagacaccaaccttaaatcctggtgaaggttacgccgaattattggctgatagaagtgcatttgaagactatgatgccgacgctgttggtgctaaaggttttggttttgtcaagttaaatcaattagcaatcgaacacttattaggtgccagatgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatctcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagtcaccggtggc

SEQ ID NO: 65 - DNA, 13921 bp, Artificial sequence
  constructed plasmid
ggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatgtccgttcaagccacaagagaagacaagtttagtttcggtttatggactgtaggttggcaagcaagagacgcattcggtgacgcaaccagaactgccttggatccagttgaagctgtccataaattggcagaaatcggtgcctacggtattacattccacgatgacgatttggttccttttggttccgatgctcaaaccagagacggtattatagccggtttcaaaaaggctttagatgaaactggtttgatcgtaccaatggttactacaaatttgtttactcatcctgtcttcaaggacggtggttttacatctaacgatagatcagtcagaagatacgctataagaaaggtattgagacaaatggatttgggtgctgaattgggtgcaaagacattagtcttgtggggtggtagagaaggtgcagaatacgattccgccaaagacgttagtgctgcattggacagatatagagaagcattgaatttgttggcacaatactctgaagatagaggttacggtttgagatttgctatagaaccaaagcctaacgaaccaagaggtgacatattgttacctactgcaggtcatgcaatcgccttcgttcaagaattggaaagaccagaattgttcggtattaatcctgaaaccggtcacgaacaaatgtctaatttgaacttcactcaaggtattgctcaagcattatggcataaaaagttgttccacatcgatttgaacggtcaacatggtccaaaattcgaccaagatttggtatttggtcacggtgacttgttgaacgctttctcattggttgatttgttggaaaacggtccagatggtgcccctgcttatgacggtccaagacattttgattacaaaccttctagaacagaagactatgatggtgtttgggaatcagcaaaggccaacatcagaatgtacttgttgttgaaggaaagagctaaggcattcagagcagatccagaagttcaagaagccttagccgcttccaaagtcgcagaattgaagacaccaaccttaaatcctggtgaaggttacgccgaattattggctgatagaagtgcatttgaagactatgatgccgacgctgttggtgctaaaggttttggttttgtcaagttaaatcaattagcaatcgaacacttattaggtgccagatgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgaggtcgacggtatcgataagcttgatatcgaattcctgcagcccgggggatccttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatggctgctaaagatgtaaagttcggtaatgatgctagagtaaaaatgttgagaggtgtaaatgtattggctgacgctgtaaaagtaactttgggtccaaaaggtagaaatgttgtcttggataagtcttttggtgctcctaccataactaaagacggtgtttcagtcgcaagagaaatcgaattggaggataagttcgaaaacatgggtgctcaaatggtcaaagaagtcgcctctaaggctaacgatgctgcaggtgacggtactacaaccgctactgttttggctcaagcaattataacagaaggtttaaaagcagttgccgctggtatgaatccaatggatttgaaaagaggtattgacaaggccgtcactgcagccgtagaagaattgaaagcattatcagtcccttgttctgattcaaaggccatcgctcaagtaggtaccatttccgctaacagtgatgaaactgttggtaaattaattgcagaagccatggacaaagtcggtaaagaaggtgtaataaccgttgaagatggtactggtttgcaagatgaattagacgtagttgagggtatgcaatttgatagaggttatttgtcaccatacttcatcaataagcctgaaacaggtgctgttgaattggaatccccttttattttgttggcagataaaaagattagtaacataagagaaatgttgccagttttagaagctgtcgcaaaagccggtaaacctttgttaatcattgctgaagatgttgaaggtgaagcattggcaacattagtcgtaaataccatgagaggtattgtaaaagttgctgcagttaaggctccaggtttcggtgacagaagaaaagctatgttgcaagacattgcaacattaaccggtggtacagttatctccgaagaaattggtatggaattggaaaaggccaccttggaagatttgggtcaagctaagagagttgtcattaataaggatactacaaccatcatcgacggtgtaggtgaagaagccgctatacaaggtagagttgctcaaataagacaacaaatcgaagaagcaacttctgattatgacagagaaaaattgcaagaaagagttgcaaagttagccggtggtgtcgctgtaattaaagttggtgcagccaccgaagtcgaaatgaaggaaaagaaagcaagagtagaagatgctttgcatgcaacaagagctgcagttgaagaaggtgtagttgcaggtggtggtgtcgccttaattagagtagcctccaaattggctgatttgagaggtcaaaatgaagaccaaaacgtaggtatcaaggttgccttaagagctatggaagcaccattgagacaaatcgttttgaactgtggtgaagaacctagtgtcgtagctaacactgttaaaggtggtgacggtaattatggttacaacgccgctacagaagaatacggtaacatgatcgatatgggtatattggacccaactaaggtcacaagatctgcattgcaatacgcagcctcagttgccggtttaatgattactacagaatgcatggttacagatttgcctaaaaacgacgctgccgacttgggtgccgcaggtggtatgggtggtatgggtggtatgggtggtatgatgtgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtactagttctagagcggccgcccgcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgattaattaattatgcttcaacaattgccaagatatctgattcagacatgatcaaaacttcttcgttatcaatcttttctgacttaacaccgtaaccatcattgaaaataacaatgtcaccaaccttaacatccaaaggcttaacttcaccgttttctaaaattctaccattaccaacagccaaaacttcacctcttgtactcttagctgcagcggaaccagtcaaaacaataccacctgcagatttggtttcaacttcctttctcttaacaataactctatcatgcaatggtctaatattcattttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactagatctatccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatctcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagtcaccggtggc

SEQ ID NO: 66 - DNA, 9684 bp, Artificial sequence
  constructed plasmid
ccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatggctgctaaagatgtaaagttcggtaatgatgctagagtaaaaatgttgagaggtgtaaatgtattggctgacgctgtaaaagtaactttgggtccaaaaggtagaaatgttgtcttggataagtcttttggtgctcctaccataactaaagacggtgtttcagtcgcaagagaaatcgaattggaggataagttcgaaaacatgggtgctcaaatggtcaaagaagtcgcctctaaggctaacgatgctgcaggtgacggtactacaaccgctactgttttggctcaagcaattataacagaaggtttaaaagcagttgccgctggtatgaatccaatggatttgaaaagaggtattgacaaggccgtcactgcagccgtagaagaattgaaagcattatcagtcccttgttctgattcaaaggccatcgctcaagtaggtaccatttccgctaacagtgatgaaactgttggtaaattaattgcagaagccatggacaaagtcggtaaagaaggtgtaataaccgttgaagatggtactggtttgcaagatgaattagacgtagttgagggtatgcaatttgatagaggttatttgtcaccatacttcatcaataagcctgaaacaggtgctgttgaattggaatccccttttattttgttggcagataaaaagattagtaacataagagaaatgttgccagttttagaagctgtcgcaaaagccggtaaacctttgttaatcattgctgaagatgttgaaggtgaagcattggcaacattagtcgtaaataccatgagaggtattgtaaaagttgctgcagttaaggctccaggtttcggtgacagaagaaaagctatgttgcaagacattgcaacattaaccggtggtacagttatctccgaagaaattggtatggaattggaaaaggccaccttggaagatttgggtcaagctaagagagttgtcattaataaggatactacaaccatcatcgacggtgtaggtgaagaagccgctatacaaggtagagttgctcaaataagacaacaaatcgaagaagcaacttctgattatgacagagaaaaattgcaagaaagagttgcaaagttagccggtggtgtcgctgtaattaaagttggtgcagccaccgaagtcgaaatgaaggaaaagaaagcaagagtagaagatgctttgcatgcaacaagagctgcagttgaagaaggtgtagttgcaggtggtggtgtcgccttaattagagtagcctccaaattggctgatttgagaggtcaaaatgaagaccaaaacgtaggtatcaaggttgccttaagagctatggaagcaccattgagacaaatcgttttgaactgtggtgaagaacctagtgtcgtagctaacactgttaaaggtggtgacggtaattatggttacaacgccgctacagaagaatacggtaacatgatcgatatgggtatattggacccaactaaggtcacaagatctgcattgcaatacgcagcctcagttgccggtttaatgattactacagaatgcatggttacagatttgcctaaaaacgacgctgccgacttgggtgccgcaggtggtatgggtggtatgggtggtatgggtggtatgatgtgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtactagttctagagcggccgcccgcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgattaattaattatgcttcaacaattgccaagatatctgattcagacatgatcaaaacttcttcgttatcaatcttttctgacttaacaccgtaaccatcattgaaaataacaatgtcaccaaccttaacatccaaaggcttaacttcaccgttttctaaaattctaccattaccaacagccaaaacttcacctcttgtactcttagctgcagcggaaccagtcaaaacaataccacctgcagatttggtttcaacttcctttctcttaacaataactctatcatgcaatggtctaatattcattttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactagatctatccgcggtggagct

SEQ ID NO: 67 - DNA, 12642 bp, Artificial sequence
  constructed plasmid
ggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatgtccgttcaagccacaagagaagacaagtttagtttcggtttatggactgtaggttggcaagcaagagacgcattcggtgacgcaaccagaactgccttggatccagttgaagctgtccataaattggcagaaatcggtgcctacggtattacattccacgatgacgatttggttccttttggttccgatgctcaaaccagagacggtattatagccggtttcaaaaaggctttagatgaaactggtttgatcgtaccaatggttactacaaatttgtttactcatcctgtcttcaaggacggtggttttacatctaacgatagatcagtcagaagatacgctataagaaaggtattgagacaaatggatttgggtgctgaattgggtgcaaagacattagtcttgtggggtggtagagaaggtgcagaatacgattccgccaaagacgttagtgctgcattggacagatatagagaagcattgaatttgttggcacaatactctgaagatagaggttacggtttgagatttgctatagaaccaaagcctaacgaaccaagaggtgacatattgttacctactgcaggtcatgcaatcgccttcgttcaagaattggaaagaccagaattgttcggtattaatcctgaaaccggtcacgaacaaatgtctaatttgaacttcactcaaggtattgctcaagcattatggcataaaaagttgttccacatcgatttgaacggtcaacatggtccaaaattcgaccaagatttggtatttggtcacggtgacttgttgaacgctttctcattggttgatttgttggaaaacggtccagatggtgcccctgcttatgacggtccaagacattttgattacaaaccttctagaacagaagactatgatggtgtttgggaatcagcaaaggccaacatcagaatgtacttgttgttgaaggaaagagctaaggcattcagagcagatccagaagttcaagaagccttagccgcttccaaagtcgcagaattgaagacaccaaccttaaatcctggtgaaggttacgccgaattattggctgatagaagtgcatttgaagactatgatgccgacgctgttggtgctaaaggttttggttttgtcaagttaaatcaattagcaatcgaacacttattaggtgccagatgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgaggtcgacggtatcgataagcttgatatcgaattcctgcagcccgggggatccttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatggctgctaaagatgtaaagttcggtaatgatgctagagtaaaaatgttgagaggtgtaaatgtattggctgacgctgtaaaagtaactttgggtccaaaaggtagaaatgttgtcttggataagtcttttggtgctcctaccataactaaagacggtgtttcagtcgcaagagaaatcgaattggaggataagttcgaaaacatgggtgctcaaatggtcaaagaagtcgcctctaaggctaacgatgctgcaggtgacggtactacaaccgctactgttttggctcaagcaattataacagaaggtttaaaagcagttgccgctggtatgaatccaatggatttgaaaagaggtattgacaaggccgtcactgcagccgtagaagaattgaaagcattatcagtcccttgttctgattcaaaggccatcgctcaagtaggtaccatttccgctaacagtgatgaaactgttggtaaattaattgcagaagccatggacaaagtcggtaaagaaggtgtaataaccgttgaagatggtactggtttgcaagatgaattagacgtagttgagggtatgcaatttgatagaggttatttgtcaccatacttcatcaataagcctgaaacaggtgctgttgaattggaatccccttttattttgttggcagataaaaagattagtaacataagagaaatgttgccagttttagaagctgtcgcaaaagccggtaaacctttgttaatcattgctgaagatgttgaaggtgaagcattggcaacattagtcgtaaataccatgagaggtattgtaaaagttgctgcagttaaggctccaggtttcggtgacagaagaaaagctatgttgcaagacattgcaacattaaccggtggtacagttatctccgaagaaattggtatggaattggaaaaggccaccttggaagatttgggtcaagctaagagagttgtcattaataaggatactacaaccatcatcgacggtgtaggtgaagaagccgctatacaaggtagagttgctcaaataagacaacaaatcgaagaagcaacttctgattatgacagagaaaaattgcaagaaagagttgcaaagttagccggtggtgtcgctgtaattaaagttggtgcagccaccgaagtcgaaatgaaggaaaagaaagcaagagtagaagatgctttgcatgcaacaagagctgcagttgaagaaggtgtagttgcaggtggtggtgtcgccttaattagagtagcctccaaattggctgatttgagaggtcaaaatgaagaccaaaacgtaggtatcaaggttgccttaagagctatggaagcaccattgagacaaatcgttttgaactgtggtgaagaacctagtgtcgtagctaacactgttaaaggtggtgacggtaattatggttacaacgccgctacagaagaatacggtaacatgatcgatatgggtatattggacccaactaaggtcacaagatctgcattgcaatacgcagcctcagttgccggtttaatgattactacagaatgcatggttacagatttgcctaaaaacgacgctgccgacttgggtgccgcaggtggtatgggtggtatgggtggtatgggtggtatgatgtgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtactagttctagagcggccgcccgcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgattaattaattatgcttcaacaattgccaagatatctgattcagacatgatcaaaacttcttcgttatcaatcttttctgacttaacaccgtaaccatcattgaaaataacaatgtcaccaaccttaacatccaaaggcttaacttcaccgttttctaaaattctaccattaccaacagccaaaacttcacctcttgtactcttagctgcagcggaaccagtcaaaacaataccacctgcagatttggtttcaacttcctttctcttaacaataactctatcatgcaatggtctaatattcattttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactagatctatccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatgatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcgccagtagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggtggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacggaattatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccgtcgacggtatcgataagcttgatatcgaattcctgcagcccgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacagatcactagtcaccggtggc

SEQ ID NO: 68 - DNA, 8848 bp, Artificial sequence
  constructed plasmid
ccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatgatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcgccagtagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggtggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacggaattatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccgtcgacggtatcgataagcttgatatcgaattcctgcagcccgggggatccttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatggctgctaaagatgtaaagttcggtaatgatgctagagtaaaaatgttgagaggtgtaaatgtattggctgacgctgtaaaagtaactttgggtccaaaaggtagaaatgttgtcttggataagtcttttggtgctcctaccataactaaagacggtgtttcagtcgcaagagaaatcgaattggaggataagttcgaaaacatgggtgctcaaatggtcaaagaagtcgcctctaaggctaacgatgctgcaggtgacggtactacaaccgctactgttttggctcaagcaattataacagaaggtttaaaagcagttgccgctggtatgaatccaatggatttgaaaagaggtattgacaaggccgtcactgcagccgtagaagaattgaaagcattatcagtcccttgttctgattcaaaggccatcgctcaagtaggtaccatttccgctaacagtgatgaaactgttggtaaattaattgcagaagccatggacaaagtcggtaaagaaggtgtaataaccgttgaagatggtactggtttgcaagatgaattagacgtagttgagggtatgcaatttgatagaggttatttgtcaccatacttcatcaataagcctgaaacaggtgctgttgaattggaatccccttttattttgttggcagataaaaagattagtaacataagagaaatgttgccagttttagaagctgtcgcaaaagccggtaaacctttgttaatcattgctgaagatgttgaaggtgaagcattggcaacattagtcgtaaataccatgagaggtattgtaaaagttgctgcagttaaggctccaggtttcggtgacagaagaaaagctatgttgcaagacattgcaacattaaccggtggtacagttatctccgaagaaattggtatggaattggaaaaggccaccttggaagatttgggtcaagctaagagagttgtcattaataaggatactacaaccatcatcgacggtgtaggtgaagaagccgctatacaaggtagagttgctcaaataagacaacaaatcgaagaagcaacttctgattatgacagagaaaaattgcaagaaagagttgcaaagttagccggtggtgtcgctgtaattaaagttggtgcagccaccgaagtcgaaatgaaggaaaagaaagcaagagtagaagatgctttgcatgcaacaagagctgcagttgaagaaggtgtagttgcaggtggtggtgtcgccttaattagagtagcctccaaattggctgatttgagaggtcaaaatgaagaccaaaacgtaggtatcaaggttgccttaagagctatggaagcaccattgagacaaatcgttttgaactgtggtgaagaacctagtgtcgtagctaacactgttaaaggtggtgacggtaattatggttacaacgccgctacagaagaatacggtaacatgatcgatatgggtatattggacccaactaaggtcacaagatctgcattgcaatacgcagcctcagttgccggtttaatgattactacagaatgcatggttacagatttgcctaaaaacgacgctgccgacttgggtgccgcaggtggtatgggtggtatgggtggtatgggtggtatgatgtgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtactagttctagagcggccgcccgcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgattaattaattatgcttcaacaattgccaagatatctgattcagacatgatcaaaacttcttcgttatcaatcttttctgacttaacaccgtaaccatcattgaaaataacaatgtcaccaaccttaacatccaaaggcttaacttcaccgttttctaaaattctaccattaccaacagccaaaacttcacctcttgtactcttagctgcagcggaaccagtcaaaacaataccacctgcagatttggtttcaacttcctttctcttaacaataactctatcatgcaatggtctaatattcattttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactagatctatccgcggtggagct

SEQ ID NO: 69 - DNA, 21 bp, Artificial sequence
  primer
agagtgcgttcaaggctcttg

SEQ ID NO: 70 - DNA, 21 bp, Artificial sequence
  primer
gagggaacatcgttggtacca

SEQ ID NO: 71 - DNA, 25 bp, Artificial sequence
  probe
ttgccataagagaagccacctcgcc

SEQ ID NO: 72 - DNA, 21 bp, Artificial sequence
  primer
ttgcgaagagcgacaaagatt

SEQ ID NO: 73 - DNA, 22 bp, Artificial sequence
  primer
ccttcatctcttccacccatgt

SEQ ID NO: 74 - DNA, 24 bp, Artificial sequence
  probe
tgttatcggctttattgctcaaag

SEQ ID NO: 75 - DNA, 24 bp, Artificial sequence
  primer
cattgcaagatgtttacaagattg

SEQ ID NO: 76 - DNA, 22 bp, Artificial sequence
  primer
tgatgacaccggtttcaactct

SEQ ID NO: 77 - DNA, 23 bp, Artificial sequence
  probe
tggtattggtactgtgccagtcg

SEQ ID NO: 78 - DNA, 26 bp, Artificial sequence
  primer
ccgtagaagaattgaaagcattatca

SEQ ID NO: 79 - DNA, 25 bp, Artificial sequence
  primer
gttagcggaaatggtacctacttga

SEQ ID NO: 80 - DNA, 26 bp, Artificial sequence
  probe
cccttgttctgattcaaaggccatcg

SEQ ID NO: 81 - DNA, 19 bp, Artificial sequence
  primer
gcagcggaaccagtcaaaa

SEQ ID NO: 82 - DNA, 30 bp, Artificial sequence
  primer
gcatgatagagttattgttaagagaaagga

SEQ ID NO: 83 - DNA, 23 bp, Artificial sequence
  probe
ccacctgcagatttggtttcaac

SEQ ID NO: 84 - DNA, 20 bp, Artificial sequence
  primer
ggcaagcaagagacgcattc

SEQ ID NO: 85 - DNA, 25 bp, Artificial sequence
  primer
aatttatggacagcttcaactggat

SEQ ID NO: 86 - DNA, 22 bp, Artificial sequence
  probe
tgacgcaaccagaactgccttg

SEQ ID NO: 87 - DNA, 16404 bp, Artificial sequence
  constructed plasmid
gatccacgatcgcattgcggattacgtattctaatgttcagtaccgttcgtataatgtatgctatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtacccgccaactctgttcgagaatgatgtaatcaagaaggtctcacaaaaccatccaggcagtaccacttcccaagtattgcttagatgggcaactcagagaggcattgccgtcattccaaaatcttccaagaaggaaaggttacttggcaacctagaaatcgaaaaaaagttcactttaacggagcaagaattgaaggatatttctgcactaaatgccaacatcagatttaatgatccatggacctggttggatggtaaattccccacttttgcctgatccagccagtaaaatccatactcaacgacgatatgaacaaatttccctcattccgatgctgtatatgtgtataaatttttacatgctcttctgtttagacacagaacagctttaaataaaatgttggatatactttttctgcctgtggtgtcatccacgcttttaattcatctcttgtatggttgacaatttggctattttttaacagaacccaacggtaattgaaattaaaagggaaacgagtgggggcgatgagtgagtgatacggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattaggcgcctacttctagggggcctatcaagtaaattactcctggtacactgaagtatataagggatatagaagcaaatagttgtcagtgcaatccttcaagacgattgggaaaatactgtaatataaatcgtaaaggaaaattggaaattttttaaagatgtcttcactggttactcttaataacggtctgaaaatgcccctagtcggcttagggtgctggaaaattgacaaaaaagtctgtgcgaatcaaatttatgaagctatcaaattaggctaccgtttattcgatggtgcttgcgactacggcaacgaaaaggaagttggtgaaggtatcaggaaagccatctccgaaggtcttgtttctagaaaggatatatttgttgtttcaaagttatggaacaattttcaccatcctgatcatgtaaaattagctttaaagaagaccttaagcgatatgggacttgattatttagacctgtattatattcacttcccaatcgccttcaaatatgttccatttgaagagaaataccctccaggattctatacgggcgcagaaggattctatacgggcgcagaactagtgatctcgaggttccagagctcggatccaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcatcccgggttagatgagagtcttttccagttcgcttaaggggacaatcttggaattatagcgatcccaattttcattatccacatcggatatgctttccattacatgccatggaaaattgtcattcagaaatttatcaaaaggaactgcaattttattagagtcatataacaatgaccacatggccttataacaaccaccaagggcacatgagtttggtgtttctagcctaaaattaccctttgtagcaccaatgacttgagcaaacttcttcacaatagcatcgtttttagaagccccacctacaaaaaaagtcctttctggccttttatttaggtagtcccgcagcggagattcatcgtaatcaaacttcacgattgtatcttcgttcagtctctgttgtgagcttgcgtttgaatccgaaagcaggggagatattcttaccctgcaacttaaagcctgtgattctacaatatttttggcatcgtgcctcttgtctttgaacttggccacctctctttcaatcatacccgtttttggattgaagataacccttttgtttatggcttttacgctaggaacgatctcccccagaggaaaatatacacctaattcattttcactactttctgagtcatctagcacagcttgattaaaaagagtccaatcgttagtcttctcataattattttcccgttctttgtttaactcgtctcttatcctctcccttgccaaagaaccattacaataacaaatcatacccatataatggtttggcagagttggatgaatgaaaagatgatagttcggagaggggtgatacttatcggtgaccagaagaactgtagtacttgttcctagggaaacgagaacgtcattcttccgcaggggtaaagaacatatagtggctaaattatccccagtcatgggagagaccttgcagtttgtattgaaaccgtacttctcaataaaatatttacagatggtacccgctatcaaatttttcatgggtgctctcattaatttttgtctgatagttttatccttagaagaactatcaattagatgtagtagctcatcactgaattttctttcacgtatatcataaaggttcataccacaggcatctgcctcctctaattcaacaagatggcccactaagatagaagtcaaaaaattagacactaaagaaatggtctttgttttttcgtaagcttctggttctaattgtgcaattttcagaatttgaggaccagtaaatctaaaatgggctctggaccctgttaattgagccattttttcaggcccacctatgcactcttcaaactcttgacattgctttgcagtactgtggtcttgccaattgggggcggtttgccttgcaaatgctacagagctcacgtagtgcaataaatctttttccggtttcttattcaattgctctaacagagattcggcttgggaggaccagtagacagacccgtgctgctggcaggaccctgagacggccataactttgttcaatggaaatttagcctcgcgatatttcgagagaaccagatctagagcctctaaccacatggctacgggacattcgatagtgtcgccgtgtatatagacacccttctttgtgtgataatgcggaagatccttttcaaattccactgtttctgaatggacaatttttaggtcctggttaatggcgagacatttcagttgttgggtcgaaagatcaaacccaagatagtatgagtctaaagacattgtgttggaaacctctcttgtctgtctctgaattactgaacacaacatactagtcgtacggttttattttttacttatattgctggtagggtaaaaaaatataactcctaggaataggttgtctatatgtttttgtcttgcttctataattgtaacaaacaaggaaagggaaaatactgggtgtaaaagccattgagtcaagttaggtcatcccttttatacaaaatttttcaattttttttccaagattcttgtacgattaattattttttttttgcgtcctacagcgtgatgaaaatttccgcctgctgcaagatgagcgggaacgggcgaaatgtgcacgcgcacaacttacgaaacgcggatgagtcactgacagccaccgcagaggttctgactcctactgagctctattggaggtggcagaaccggtaccggaggagaccgctataaccggtttgaatttattgtcacagtgtcacatcagcggcaactcagaagtttgacagcaagcaagttcatcattcgaactagccttattgttttagttcagtgacagcgaactgccgtactcgatgctttatttctcacggtagagcggaagaacagataggggcagcgtgagaagagttagaaagtaaatttttatcacgtctgaagtattcttattcataggaaattttgcaaggttttttagctcaataacgggctaagttatataaggtgttcacgcgattttcttgttatgtatacctcttctggcgcgcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacacgtacgactagtatgactcaattcactgacattgataagttggccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaaattctgatcgtagatcatcagatttgatatgatattatttgtgaaaaaatgaaataaaactttatacaacttaaatacaactttttttataaacgattaagcaaaaaaatagtttcaaacttttaacaatattccaaacactcagtccttttccttcttatattataggtgtacgtattatagaaaaatttcaatgattactttttctttctttttccttgtaccagcacatggccgagcttgaatgttaaacccttcgagagaatcacaccattcaagtataaagccaataaagaatataactcctaaaaggctaattgaaaccctgtgatttttgcccgggtttaaggcgcgccctttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccactagcatggctgccggtgtcccaaaaattgatgcgttagaatctttgggcaatcctttggaggatgccaagagagctgcagcatacagagcagttgatgaaaatttaaaatttgatgatcacaaaattattggaattggtagtggtagcacagtggtttatgttgccgaaagaattggacaatatttgcatgaccctaaattttatgaagtagcgtctaaattcatttgcattccaacaggattccaatcaagaaacttgattttggataacaagttgcaattaggctccattgaacagtatcctcgcattgatatagcgtttgacggtgctgatgaagtggatgagaatttacaattaattaaaggtggtggtgcttgtctatttcaagaaaaattggttagtactagtgctaaaaccttcattgtcgttgctgattcaagaaaaaagtcaccaaaacatttaggtaagaactggaggcaaggtgttcccattgaaattgtaccttcctcatacgtgagggtcaagaatgatctattagaacaattgcatgctgaaaaagttgacatcagacaaggaggttctgctaaagcaggtcctgttgtaactgacaataataacttcattatcgatgcggatttcggtgaaatttccgatccaagaaaattgcatagagaaatcaaactgttagtgggcgtggtggaaacaggtttattcatcgacaacgcttcaaaagcctacttcggtaattctgacggtagtgttgaagttaccgaaaagtgagcggccgcgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagatgcagtaatatacacagattcccgcggacgtgggaaggaaaaaattagataacaaaatctgagtgatatggaaattccgctgtatagctcatatctttccctccaccgcggtggtcgactttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaacttaagactcgagatggtcaaaccaattatagctcccagtatccttgcttctgacttcgccaacttgggttgcgaatgtcataaggtcatcaacgccggcgcagattggttacatatcgatgtcatggacggccattttgttccaaacattactctgggccaaccaattgttacctccctacgtcgttctgtgccacgccctggcgatgctagcaacacagaaaagaagcccactgcgttcttcgattgtcacatgatggttgaaaatcctgaaaaatgggtcgacgattttgctaaatgtggtgctgaccaatttacgttccactacgaggccacacaagaccctttgcatttagttaagttgattaagtctaagggcatcaaagctgcatgcgccatcaaacctggtacttctgttgacgttttatttgaactagctcctcatttggatatggctcttgttatgactgtggaacctgggtttggaggccaaaaattcatggaagacatgatgccaaaagtggaaactttgagagccaagttcccccatttgaatatccaagtcgatggtggtttgggcaaggagaccatcccgaaagccgccaaagccggtgccaacgttattgtcgctggtaccagtgttttcactgcagctgacccgcacgatgttatctccttcatgaaagaagaagtctcgaaggaattgcgttctagagatttgctagattagacgtctgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacgtacgactagtatgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttagtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaactactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcg

SEQ ID NO: 88 - DNA, 95 bp, Artificial sequence
  as a URA3 deletion scar in the genome -After removal of the KanMX marker using the cre recombinase, a 95 bp sequence consisting of a loxP site flanked by the primer binding sites remained
gcattgcggattacgtattctaatgttcagataacttcgtatagcatacattatacgaagttatccagtgatgatacaacgagttagccaaggtg

SEQ ID NO: 89 - DNA, 100 bp, Saccharomyces cerevisiae
gtccataaagcttttcaattcatcttttttttttttgttcttttttttgattccggtttctttgaaatttttttgattcggtaatctccgagcagaagga

SEQ ID NO: 90 - DNA, 100 bp, Saccharomyces cerevisiae
aaaactgtattataagtaaatgcatgtatactaaactcacaaattagagcttcaatttaattatatcagttattacccgggaatctcggtcgtaatgatt

SEQ ID NO: 91 - DNA, 100 bp, saccharomyces cerevisiae
attggcattatcacataatgaattatacattatataaagtaatgtgatttcttcgaagaatatactaaaaaatgagcaggcaagataaacgaaggcaaag

SEQ ID NO: 92 - DNA, 100 bp, Saccharomyces cerevisiae
tagtgacaccgattatttaaagctgcagcatacgatatatatacatgtgtatatatgtatacctatgaatgtcagtaagtatgtatacgaacagtatgat

SEQ ID NO: 93 - DNA, 6728 bp, Artificial sequence
  constructed vector
acatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaatgtccaatttactgcccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtgttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggat

SEQ ID NO: 94 - DNA, 9353 bp, Artificial sequence
  constructed plasmid
ccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaactagtatggctgctaaagatgtaaagttcggtaatgatgctagagtaaaaatgttgagaggtgtaaatgtattggctgacgctgtaaaagtaactttgggtccaaaaggtagaaatgttgtcttggataagtcttttggtgctcctaccataactaaagacggtgtttcagtcgcaagagaaatcgaattggaggataagttcgaaaacatgggtgctcaaatggtcaaagaagtcgcctctaaggctaacgatgctgcaggtgacggtactacaaccgctactgttttggctcaagcaattataacagaaggtttaaaagcagttgccgctggtatgaatccaatggatttgaaaagaggtattgacaaggccgtcactgcagccgtagaagaattgaaagcattatcagtcccttgttctgattcaaaggccatcgctcaagtaggtaccatttccgctaacagtgatgaaactgttggtaaattaattgcagaagccatggacaaagtcggtaaagaaggtgtaataaccgttgaagatggtactggtttgcaagatgaattagacgtagttgagggtatgcaatttgatagaggttatttgtcaccatacttcatcaataagcctgaaacaggtgctgttgaattggaatccccttttattttgttggcagataaaaagattagtaacataagagaaatgttgccagttttagaagctgtcgcaaaagccggtaaacctttgttaatcattgctgaagatgttgaaggtgaagcattggcaacattagtcgtaaataccatgagaggtattgtaaaagttgctgcagttaaggctccaggtttcggtgacagaagaaaagctatgttgcaagacattgcaacattaaccggtggtacagttatctccgaagaaattggtatggaattggaaaaggccaccttggaagatttgggtcaagctaagagagttgtcattaataaggatactacaaccatcatcgacggtgtaggtgaagaagccgctatacaaggtagagttgctcaaataagacaacaaatcgaagaagcaacttctgattatgacagagaaaaattgcaagaaagagttgcaaagttagccggtggtgtcgctgtaattaaagttggtgcagccaccgaagtcgaaatgaaggaaaagaaagcaagagtagaagatgctttgcatgcaacaagagctgcagttgaagaaggtgtagttgcaggtggtggtgtcgccttaattagagtagcctccaaattggctgatttgagaggtcaaaatgaagaccaaaacgtaggtatcaaggttgccttaagagctatggaagcaccattgagacaaatcgttttgaactgtggtgaagaacctagtgtcgtagctaacactgttaaaggtggtgacggtaattatggttacaacgccgctacagaagaatacggtaacatgatcgatatgggtatattggacccaactaaggtcacaagatctgcattgcaatacgcagcctcagttgccggtttaatgattactacagaatgcatggttacagatttgcctaaaaacgacgctgccgacttgggtgccgcaggtggtatgggtggtatgggtggtatgggtggtatgatgtgagcggccgcacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacaggcgcgcctcacttttcgatgacagccaaaacatctctagcggacaagaccaagtattcttcaccagcgtacttgacttcagtaccaccgtacttagagtacaagacaacgtcaccgaccttaacgtccaatgggactctgttacccttatcgtcgattctacctggaccgacagccaaaacagtaccttcttgtggcttttccttagcggtgtctgggataacgataccagaagcggtagtggtttcagcttcgttagcttgaacaacgattctgtcttccaatggcttgatagcgaccttagtagcggtggtgactggcatactgtttaaactttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactagatctatccgcggtggagct

SEQ ID NO: 95 - DNA, 9353 bp, Artificial sequence
  constructed plasmid
ccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaactagtatggctaagatcatcgctttcgacgaagaagctagaagaggtttggaaagaggtatgaaccaattggctgacgctgttaaggtcactttgggtccaaagggtagaaacgttgtcttggaaaagaagtggggtgctccaactatcaccaacgatggtgtctctatcgctaaggaaatcgaattggaagactcctacgaaaagatcggtgctgaattggtcaaggaagttgctaagaagactgacgatgtcgctggtgacggtactactaccgctaccgtcttggctcaagctttggttagagaaggtttgagaaacgttgctgctggtgctaacccaatggctttgaagagaggtatcgaagctgctgtcgcttctgtttccgaaggtttgcaacaattggctaaggacgttgaaactaaggaacaaatcgcttctaccgcttctatctctgctggtgactccactgtcggtgaaatcatcgctgaagctatggacaaggttggtaaagaaggtgtcatcactgttgaagaatctaacaccttcggtttggaattggaattgactgaaggtatgagattcgataagggttacatctccgcttacttcatgaccgacgctgaaagaatggaagctgtcttcgacgatccatacatcttgatcgctaactctaagatctccgctgtcaaggacttgttgccaatcttggaaaaggttatgcaatctggtaaaccattggtcatcatcgctgaagacgttgaaggtgaagctttggctactttggttgtcaacaaggttagaggtactttcaagtctgtcgctgttaaggctccaggtttcggtgacagaagaaaggctatgttggaagacatcgctatcttgactggtggtgctgtcatctctgaagaagttggtttgaagttggatgctgctgacttgtccttgttgggtcaagctagaaaggttgtcatcaccaaggatgaaactaccgttgttgacggtgctggtaacggtgaacaaatccaaggtagagttaaccaaatcagagctgaaatcgaaagatctgactccgattacgacagagaaaagttgcaagaaagattggctaagttggctggtggtgtcgctgttatcaaggtcggtgctgctaccgaagttgaattgaaggaaagaaagcacagaatcgaagacgctgtcagaaacgctaaggctgctgtcgaagaaggtatcgttccaggtggtggtgtcgctttggttcaagctggtaaaactgctttcgataagttggacttggttggtgacgaagctaccggtgctaacatcgtcaaggttgctttggacgctccattgagacaaatcgctgtcaacgctggtttggaaggtggtgttgtcgttgaaaaggttagaaacttgtctgctggtcacggtttgaacgctgctactggtgaatacgtcgatttgttggctgctggtatcatcgacccagctaaggttaccagatctgctttgcaaaacgctgcttccatcgctgctttgttcttgactaccgaagctgtcgttgctgacaagccagaaaagaacccagctccagctggtgctccaggtggtggtgacatggacttctgagcggccgcacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacaggcgcgcctcacaagtacaaaccagtaccatcggattcaactctgttagcagcaacagcgtgaacgtctctttctctcaacaaaacgtattctttaccgtgcaattcgacttcagatctatcgtctggatcgaacaaaactctgtcaccgacaacgatggatctgacgtttggaccaacaccgacagcaacagcccaagacaatcttctaccgatagtagcggtagctgggatgacgataccagcggaagatcttctttcaccttcaccaccatcttgtctgaccaaaactctatcgtgcaacattctgattggcaaaccagcatcggttctagtatcagcggacatactgtttaaactttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactagatctatccgcggtggagct

SEQ ID NO: 96 - protein, 439 aa, Ruminococcus flavefaciens
MEFFKNISKIPYEGKDSTNPLAFKYYNPDEVIDGKKMRDIMKFALSWWHTMGGDGTDMFGCGTADKTWGENDPAARAKAKVDAAFEIMQKLSIDYFCFHDRDLSPEYGSLKDTNAQLDIVTDYIKAKQAETGLKCLWGTAKCFDHPRFMHGAGTSPSADVFAFSAAQIKKALESTVKLGGTGYVFWGGREGYETLLNTNMGLELDNMARLMKMAVEYGRSIGFKGDFYIEPKPKEPTKHQYDFDTATVLGFLRKYGLDKDFKMNIEANHATLAQHTFQHELCVARTNGAFGSIDANQGDPLLGWDTDQFPTNIYDTTMCMYEVIKAGGFTNGGLNFDAKARRGSFTPEDIFYSYIAGMDAFALGYKAASKLIADGRIDSFISDRYASWSEGIGLDIISGKADMAALEKYALEKGEVTDSISSGRQELLESIVNNVIFNL

SEQ ID NO: 97 - protein, 441 aa, Ruminococcus champanellensis
MSEFFTGISKIPFEGKASNNPMAFKYYNPDEVVGGKTMREQLKFALSWWHTMGGDGTDMFGVGTTNKKFGGTDPMDIAKRKVNAAFELMDKLSIDYFCFHDRDLAPEADNLKETNQRLDEITEYIAQMMQLNPDKKVLWGTANCFGNPRYMHGAGTAPNADVFAFAAAQIKKAIEITVKLGGKGYVFWGGREGYETLLNTNMGLELDNMARLLHMAVDYARSIGFTGDFYIEPKPKEPTKHQYDFDTATVIGFLRKYNLDKDFKMNIEANHATLAQHTFQHELRVARENGFFGSIDANQGDTLLGWDTDQFPTNTYDAALCMYEVLKAGGFTNGGLNFDSKARRGSFEMEDIFHSYIAGMDTFALGLKIAQKMIDDGRIDQFVADRYASWNTGIGADIISGKATMADLEAYALSKGDVTASLKSGRQELLESILNNIMFNL

SEQ ID NO: 98 - protein, 439 aa, Unknown
  uncultured bacteria from cow rumen
MGEIFSNIPVIKYEGPDSKNPLAFKYYDPERVILGKKMKEHLPFAMAWWHNLCANGVDMFGRGTIDKLFGAAEAGTMEHAKAKVDAGIEFMQKLGIEYYCFHDVDLVPEADDINETNRRLDELTDYLKEKTAGTNIKCLWGTANMFSNPRFMNGAGSTNDVDVYCFAAAQVKKAIEMTVKLGGRGYVFWGGREGYETLLNTKVQMELENIANLMKMARDYGRSIGFKGTFLIEPKPKEPMKHQYDYDAATAIGFLRQYGLDQDFKMNIEANHATLAGHTFQHELRISRINGMLGSIDANQGDIMLGWDTDCFPSNVYDTTLAMYEIVRNGGLPVGINFDSKNRRPSNTYEDMFHAFILGMDSFAFGLIKAAQIIEDGRIEGFTEKKYESFNTELGQKIRKGEATLEELAAHAADLKAPKVPVSGRQEYLEGVLNNIILS

SEQ ID NO: 99 - DNA, 1317 bp, artificial sequence
  coding region for Ru2 optimized for expression in Saccharomyces cerevisiae
atgggtgaaatcttctctaacatcccagtcatcaagtacgaaggtccagactctaagaacccattggctttcaagtactacgatccagaaagagtcatcttgggtaaaaagatgaaggaacacttgccattcgctatggcttggtggcacaacttgtgtgctaacggtgttgacatgttcggtagaggtactatcgataagttgttcggtgctgctgaagctggtactatggaacacgctaaggctaaggttgacgctggtatcgagttcatgcaaaagttgggtatcgaatactactgtttccacgacgttgatttggtcccagaagctgacgatatcaacgaaaccaacagaagattggacgaattgactgattacttgaaggaaaagaccgctggtactaacatcaagtgtttgtggggtactgctaacatgttctctaacccaagattcatgaacggtgctggttccactaacgacgttgatgtctactgtttcgctgctgctcaagttaagaaggctatcgaaatgaccgtcaagttgggtggtagaggttacgttttctggggtggtagagaaggttacgaaaccttgttgaacactaaggtccaaatggaattggaaaacatcgctaacttgatgaagatggctagagactacggtagatctatcggtttcaagggtactttcttgatcgaaccaaagccaaaggaaccaatgaagcaccaatacgactacgatgctgctactgctatcggtttcttgagacaatacggtttggaccaagatttcaagatgaacatcgaagctaaccacgctaccttggctggtcacactttccaacacgaattgagaatctctagaatcaacggtatgttgggttccatcgacgctaaccaaggtgacatcatgttgggttgggacaccgattgtttcccatctaacgtttacgacaccactttggctatgtacgaaatcgttagaaacggtggtttgccagtcggtatcaacttcgactctaagaacagaagaccatccaacacttacgaagacatgttccacgctttcatcttgggtatggactctttcgctttcggtttgatcaaggctgctcaaatcatcgaagacggtagaatcgaaggtttcaccgaaaagaagtacgaatccttcaacactgaattgggtcaaaagatcagaaagggtgaagctactttggaagaattggctgctcacgctgctgacttgaaggctccaaaggttccagtctctggtagacaagaatacttggaaggtgttttgaacaacatcatcttgtcc

SEQ ID NO: 100 - protein, 395 aa, Unknown
  uncultured bacteria from cow rumen
MAWWHNMCANGKDMFGTGTADKSFGAEPGTMEHAKAKVDAAIEFMQKLGIEYYCFHDVDLVPEDEDDINVTNARLDEISDYILEKTKGTNIRCLWGTANMFNNPRFMNGAGSTNSADVYCFAAAQIKKALDITVKLGGRGYVFWGGREGYETLLNTDVKLEQENIANLMHMAVEYGRSIGFKGDFLIEPKPKEPMKHQYDFDAATAIGFLRQYGLDKDFKLNIEANHATLAGHTFQHELRISAMNGMLGSIDANQGDMLLGWDTDEFPFNVYDTTLAMYEVLKAGGINGGFNFDSKNRRPSNTYEDMFYGYILGMDSFALGLIKAAAIIEDGRIEKQLADRYSSYSNTEIGKKIRNHTATLKELAEYAATLKKPGDPGSGRQELLEQIMNEVMFG

SEQ ID NO: 101 - DNA, 1185 bp, artificial sequence
  coding region for Ru3 optimized for expression in Saccharomyces cerevisiae
atggcttggtggcacaacatgtgtgctaacggcaaggatatgttcggtactggtactgctgataagtctttcggtgctgaaccaggcaccatggaacacgctaaggctaaggttgacgctgctatcgagttcatgcaaaagttgggtatcgaatactactgtttccacgacgttgatttggtcccagaagacgaagacgatatcaacgtcactaacgctagattggacgaaatctctgattacatcttggaaaagaccaagggtactaacatcagatgtttgtggggtactgctaacatgttcaacaacccaagattcatgaacggtgctggttctactaactccgctgacgtttactgtttcgctgctgctcaaatcaagaaggctttggacatcaccgttaagttgggtggtagaggttacgtcttctggggtggtagagaaggttacgaaaccttgttgaacactgacgttaagttggaacaagaaaacatcgctaacttgatgcacatggctgtcgaatacggtagatctatcggtttcaagggtgacttcttgatcgaaccaaagccaaaggaaccaatgaagcaccaatacgacttcgatgctgctactgctatcggtttcttgagacaatacggtttggacaaggatttcaagttgaacatcgaagctaaccacgctaccttggctggtcacactttccaacacgaattgagaatctctgctatgaacggtatgttgggttccatcgacgctaaccaaggtgacatgttgttgggttgggacaccgatgaatttccattcaacgtttacgacaccactttggctatgtacgaagtcttgaaggctggtggtatcaacggtggtttcaacttcgactctaagaacagaagaccatccaacacttacgaagacatgttctacggttacatcttgggtatggattctttcgctttgggtttgatcaaggctgctgctatcatcgaagacggtagaatcgaaaagcaattggctgatagatactcttcctactccaacaccgaaatcggtaaaaagatcagaaaccacaccgctactttgaaggaattggctgaatacgctgctactttgaagaagccaggtgacccaggttccggtagacaagaattgttggaacaaatcatgaacgaagttatgttcggt

(19) United States
(12) Patent Application Publication
(10) Pub. No.: US 2014/0178954 A1
(43) Pub. Date: Jun. 26, 2014

(54) EXPRESSION OF XYLOSE ISOMERASE ACTIVITY IN YEAST

(71) Applicant: E I DU PONT DE NEMOURS AND COMPANY, Wilmington, DE (US)

(72) Inventors: WILLIAM D HITZ, Wilmington, DE (US); Min Qi, Hockessin, DE (US); Sarah Eve Rush, Hockessin, DE (US); Luan Tao, Wallingford, PA (US); Paul V. Viitanen, West Chester, PA (US); Jianjun Yang, Hockessin, DE (US); Rick W. Ye, Hockessin, DE (US)

(73) Assignee: E I Du Pont De Nemours and Company, Wilmington, DE (US)

(21) Appl. No.: 13/792,668

(22) Filed: Mar. 11, 2013

Related U.S. Application Data
(60) Provisional application No. 61/739,755, filed on Dec. 20, 2012.

(51) Int. Cl. C12N 15/81

(52) U.S. Cl. CPC C12N 15/81
USPC 435/158; 435/254.2; 435/471; 435/161; 435/160

(57) ABSTRACT

Expression of a xylose isomerase in a yeast cell that expresses the chaperonins GroES and GroEL was found to result in enzymatically active xylose isomerase, while there is little to no activity with expression of the bacterial xylose isomerase in a yeast cell lacking GroES and GroEL. A yeast cell expressing xylose isomerase activity, and a complete xylose utilization pathway, provides a yeast cell that can produce a target compound, such as ethanol, butanol, or 1,3-propanediol, using xylose derived from lignocellulosic biomass as a carbon source.

This application claims the benefit of U.S. Provisional Application 61/739,755, filed Dec. 20, 2012 and is incorporated by reference in its entirety.

### FIELD OF THE INVENTION

The invention relates to the field of genetic engineering of yeast. More specifically, Saccharomyces cerevisiae is engineered to express an active xylose isomerase enzyme by also expressing GroES and GroEL proteins, and thus can grow on xylose as the sole carbohydrate when the rest of the xylose-utilization pathway is active.

### BACKGROUND OF THE INVENTION

Currently fermentative production of ethanol is typically by yeasts, particularly Saccharomyces cerevisiae, using hexoses obtained from grains or mash as the carbohydrate source. Use of hydrolysate prepared from cellulosic biomass as a carbohydrate source for fermentation is desirable, as this is a readily renewable resource that does not compete with the food supply. After glucose, the second most abundant sugar in cellulosic biomass is xylose, a pentose. Saccharomyces cerevisiae is not naturally capable of metabolizing xylose, but can be engineered to metabolize xylose with expression of xylose isomerase activity to convert xylose to xylulose, and additional pathway engineering.

Success in expressing heterologous xylose isomerase enzymes that are active in yeast has been limited. Expression of xylose isomerase activity in S. cerevisiae was disclosed in U.S. Pat. No. 7,622,284 and US 20110318790. However many bacterial xylose isomerases do not provide significant amounts of catalytically active enzyme when expressed in yeast, as reported in Sarthy et. al. ((1987) Appl. Environ. Microbiol. 53: 1996-2000), Amore et al. ((1989) Appl. Environ. Microbiol. 30: 351-357), and Gardonyi et al. ((2003) Enzyme and Microbial Technology. 32: 252-259).

Chaperones, which include the chaperonins, are proteins that assist in the post-translational folding of a wide variety of proteins (reviewed in Hartl and Hayer-Hartl (2002) Science 295: 1852-1858). A proteomewide analysis of E. coli identified about 85 proteins that require the GroEL/GroES chaperonins for proper folding in vivo, called Class III proteins (Kerner et al. (2005) Cell 122:209-220). Xylose isomerase was predicted to belong to this Class III. E. coli xylose isomerase was found in a soluble fraction when expressed in S. cerevisiae along with E. coli GroEL and GroES (Hung-Chun Wang, PhD Thesis (2006) Ludwig-Maximilians-Universität München).

There remains a need for additional engineered yeast cells that express xylose isomerase activity for successful utilization of xylose, thereby allowing effective use of sugars from cellulosic biomass during fermentation.

### SUMMARY OF THE INVENTION

The invention provides recombinant yeast cells that are engineered to express chaperonins and bacterial xylose isomerase, and therefore have xylose isomerase enzyme activity to enable the utilization of xylose as a carbon source.

Accordingly, the invention provides a recombinant yeast cell comprising:

a) at least one gene encoding each amino acid sequence of an interacting pair of Group I chaperonin polypeptides; and

b) at least one gene encoding a bacterial xylose isomerase polypeptide;

wherein:

i) the interacting pair of Group I chaperonins are active in the cytosol of the cell;

ii) the xylose isomerase polypeptide is converted to an active xylose isomerase enzyme; and

iii) the specific activity of the xylose isomerase enzyme is higher as compared with the specific activity of the same xylose isomerase enzyme expressed in the absence of the interacting pair of Group I chaperonin polypeptides.

In another aspect the invention provides a method for producing a yeast strain that has xylose isomerase activity comprising:
a) providing a yeast cell;
b) introducing a heterologous nucleic acid molecule encoding a GroEL polypeptide and a heterologous nucleic acid molecule encoding a GroES polypeptide; and
c) introducing a heterologous nucleic acid molecule encoding a bacterial xylose isomerase polypeptide;

wherein:
i) the GroEL and GroES polypeptides are expressed in the cytosol of the cell;
ii) the xylose isomerase polypeptide is converted to an active xylose isomerase enzyme; and
iii) the specific activity of the xylose isomerase enzyme is higher as compared with the specific activity of the same xylose isomerase enzyme expressed in the absence of the GroEL and GroES polypeptides.

In yet another aspect the invention provides a method for expressing an active bacterial xylose isomerase enzyme in yeast comprising:
a) providing a recombinant yeast cell described above; and
b) growing the yeast cell of a) whereby xylose isomerase polypeptide is converted to an active xylose isomerase enzyme.

### BRIEF DESCRIPTION OF THE FIGURES AND SEQUENCE DESCRIPTIONS

FIG. 1A shows a plasmid map of pHR81-AMXA.

FIG. 1B shows a plasmid map of pHR81-AMXA-GELS.

FIG. 2 shows a plasmid map of pRS423-GELS.

FIG. 3A shows a plasmid map of pRS313-AMXA-GELS.

FIG. 3B shows a plasmid map of pRS313-GELS.

The invention can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

TABLE 1
SEQ ID NOs for GroEL polypeptides, and coding regions that
are codon optimized for expression in S. cerevisiae

| | SEQ ID NO: | SEQ ID NO: |
|---|---|---|
| Organism | amino acid | nucleotide codon opt. |
| E. coli | 1 | 2 |
| Actinoplanes missouriensis 1 | 3 | 4 |
| Actinoplanes missouriensis 2 | 5 | 6 |
| Bacteroides thetaiotaomicron | 7 | 8 |
| Bacillus subtilis | 9 | 10 |
| Ruminococcus champanellensis | 11 | 12 |
| Zymomonas mobilis | 13 | 14 |

TABLE 2
SEQ ID NOs for GroES polypeptides, and coding regions that
are codon optimized for expression in S. cerevisiae

| | SEQ ID NO: | SEQ ID NO: |
|---|---|---|
| Organism | amino acid | nucleotide codon opt. |
| E. coli | 15 | 16 |
| Actinoplanes missouriensis 1 | 17 | 18 |
| Actinoplanes missouriensis 2 | 19 | 20 |
| Bacteroides thetaiotaomicron | 21 | 22 |
| Bacillus subtilis | 23 | 24 |
| Ruminococcus champanellensis | 25 | 26 |
| Zymomonas mobilis | 27 | 28 |

TABLE 3
SEQ ID NOs for xylose isomerase polypeptides, and coding regions
that are codon optimized for expression in S. cerevisiae

| | SEQ ID NO: | SEQ ID NO: |
|---|---|---|
| Organism | amino acid | nucleotide codon opt. |
| Actinoplanes missouriensis | 29 | 30, 59* |
| E. coli | 31 | 32, 60* |
| Bacillus subtilis | 33 | 34 |
| Streptomyces rubiginosus | 35 | 36 |
| Burkholderia phytofirmans | 37 | 38 |
| Burkholderia phymatum | 39 | 40 |
| Citrobacter youngae | 41 | 42 |
| Escherichia blattae | 43 | 44 |
| Pseudomonas fluorescens | 45 | 46 |
| Photobacterium profundum | 47 | 48 |
| Pantoea stewartii | 49 | 50 |
| Plautia stali symbiont | 51 | 52 |
| Pseudomonas syringae | 53 | 54 |
| Vibrio sp. XY-214 | 55 | 56 |
| Yokenella regensburgei | 57 | 58 |

*Two different codon-optimized sequences for the same amino acid sequence
SEQ ID NO: 61 is the nucleotide sequence of a chimeric AMxylA expression cassette.
SEQ ID NO: 62 is the nucleotide sequence of a chimeric ECgroES expression cassette.
SEQ ID NO: 63 is the nucleotide sequence of a chimeric ECgroEL expression cassette.
SEQ ID NO: 64 is the nucleotide sequence of pHR81-AMXA.
SEQ ID NO: 65 is the nucleotide sequence of pHR81-AMXA-GELS.
SEQ ID NO: 66 is the nucleotide sequence of pRS423-GELS.
SEQ ID NO: 67 is the nucleotide sequence of pRS313-AMXA-GELS.
SEQ ID NO: 68 is the nucleotide sequence of pRS313-GELS.
SEQ ID NOs: 68-86 are the nucleotide sequences of primers and probes.
SEQ ID NO: 87 is the nucleotide sequence of P5 Integration Vector.
SEQ ID NO: 88 is the nucleotide sequence of a URA3 deletion scar.
SEQ ID NO: 89 is the nucleotide sequence of the upstream ura3Δ post deletion region.
SEQ ID NO: 90 is the nucleotide sequence of the downstream ura3Δ post deletion region.
SEQ ID NO: 91 is the nucleotide sequence of the upstream his3Δ post deletion region.
SEQ ID NO: 92 is the nucleotide sequence of the downstream his3Δ post deletion region.
SEQ ID NO: 93 is the nucleotide sequence of pJT254.
SEQ ID NO: 94 is the nucleotide sequence of pRS423 Am 104GroES 550 GroEL
SEQ ID NO: 95 is the nucleotide sequence of pRS423 Am 112GroES 540 GroEL.
SEQ ID NO: 96 is the amino acid sequence of the xylose isomerase from Ruminococcus flavefaciens FD-1.
SEQ ID NO: 97 is the amino acid sequence of the xylose isomerase from Ruminococcus champanellensis 18P13.
SEQ ID NO: 98 is the amino acid sequence of Ru2.
SEQ ID NO: 99 is the nucleotide sequence of xylA(Ru2), the codon optimized coding region for Ru2.
SEQ ID NO: 100 is the amino acid sequence of Ru3.
SEQ ID NO: 101 is the nucleotide sequence of xylA(Ru3), the codon optimized coding region for Ru3.

### DETAILED DESCRIPTION

The following definitions may be used for the interpretation of the claims and specification:

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “chaperones” refers to proteins that assist in folding of certain newly synthesized proteins to prevent misfolding and aggregation.

The term “chaperonins” refers to a class of chaperones that are large double-ring complexes of about 800-1,000 kD enclosing a central cavity. There are two groups of chaperonins with similar architecture but distantly related sequence: Group I and Group II.

The term “Group I chaperonins” refers to a group of chaperonins which includes the GroELs or Hsp60s, which are found in eubacteria, and in mitochondria and chloroplasts of eukaryotic cells. These proteins interact with cofactors referred to as GroES or Hsp10. Together a GroEL or Hsp60 protein and a GroES or Hsp10 protein interact to form an active chaperonin complex are referred to herein as an “interacting pair” of Group I chaperonin polypeptides.

The term “Group II chaperonins” refers to a group of chaperonins found in archaeal bacteria and the eukaryotic cytosol, which are GroES and Hsp10 independent. An example is TRiC (TCP-1 ring complex, also called CCT for chaperonin-containing TCP-1).

The term “xylose isomerase” refers to an enzyme that catalyzes the interconversion of D-xylose and D-xylulose. Xylose isomerases (XI) belong to the group of enzymes classified as EC 5.3.1.5.

The term “Group I xylose isomerase” refers herein to a xylose isomerase (XI) protein that belongs to Group I as defined by at least one of the following criteria: a) it falls within a 50% threshold sequence identity grouping that includes the A. missouriensis XI that is prepared using molecular phylogenetic bioinformatics analysis as in Example 4 of US 20110318801, which is incorporated herein by reference; b) it substantially fits the amino acids for Group I in the specificity determining positions (SDP) identified using GroupSim analysis of the Group I and Group II XI sets determined from molecular phylogenetic analysis that are given in Table 6 in Example 4 of US 20110318801; and/or c) it has an E-value of 1E-15 or less when queried using a Profile Hidden Markov Model prepared using SEQ ID NOs: 2, 24, 32, 34, 42, 54, 66, 68, 78, 96, 100, 106, 108, 122, 126, 128, 130, 132, 135, 137, and 142 of US 20110318801; where the query is carried out using the hmmsearch algorithm with the Z parameter set to 1 billion, as in Example 4 of US 20110318801. It is understood that although “Group 1” xylose isomerases are known and defined in the literature that the definition provided herein is more precise than the literature definition and is the definition that informs the following discussion.

The term “Group II xylose isomerase” refers herein to a xylose isomerase (XI) protein that belongs to Group II as defined in the art, such as in Park and Batt ((2004) Applied and Environmental Microbiology 70:4318-4325), wherein Group II XIs are distinguished from Group I XIs in being typically longer than Group I XIs: about 440 to 460 amino acids vs about 380 to 390 amino acids, respectively. Group II XIs have only 20-30% amino acid identity with Group I XIs, while among Group I XIs there is amino acid identity of at least about 50%. Analysis of Group I and Group II XIs is more fully disclosed in US 20110318801, which includes a phylogenetic tree.

The term “E-value”, as known in the art of bioinformatics, is “Expect-value” which provides the probability that a match will occur by chance. It provides the statistical significance of the match to a sequence. The lower the E-value, the more significant the hit.

The term “gene” refers to a nucleic acid fragment that expresses a specific protein or functional RNA molecule, which may optionally include regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

The term “promoter” or “Initiation control regions” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”.

The term “expression”, as used herein, refers to the transcription and stable accumulation of coding (mRNA) or functional RNA derived from a gene. Expression may also refer to translation of mRNA into a polypeptide. “Overexpression” refers to the production of a gene product in transgenic organisms that exceeds levels of production in normal or non-transformed organisms.

The term “transformation” as used herein, refers to the transfer of a nucleic acid fragment into a host organism, resulting in genetically stable inheritance. The transferred nucleic acid may be in the form of a plasmid maintained in the host cell, or some transferred nucleic acid may be integrated into the genome of the host cell. Host organisms containing the transformed nucleic acid fragments are referred to as “transgenic” or “recombinant” or “transformed” organisms.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “selectable marker” means an identifying factor, usually an antibiotic or chemical resistance gene, that is able to be selected for based upon the marker gene's effect, i.e., resistance to an antibiotic, wherein the effect is used to track the inheritance of a nucleic acid of interest and/or to identify a cell or organism that has inherited the nucleic acid of interest.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “carbon substrate” or “fermentable carbon substrate” refers to a carbon source capable of being metabolized by microorganisms. A type of carbon substrate is “fermentable sugars” which refers to oligosaccharides and monosaccharides that can be used as a carbon source by a microorganism in a fermentation process.

The term “lignocellulosic” refers to a composition comprising both lignin and cellulose. Lignocellulosic material may also comprise hemicellulose.

The term “cellulosic” refers to a composition comprising cellulose and additional components, which may include hemicellulose and lignin.

The term “saccharification” refers to the production of fermentable sugars from polysaccharides.

The term “pretreated biomass” means biomass that has been subjected to thermal, physical and/or chemical pretreatment to increase the availability of polysaccharides in the biomass to saccharification enzymes.

“Biomass” refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn cobs, crop residues such as corn husks, corn stover, grasses, wheat, wheat straw, barley straw, hay, rice straw, switchgrass, waste paper, sugar cane bagasse, sorghum, components obtained from milling of grains, trees, branches, roots, leaves, wood chips, sawdust, shrubs and bushes, vegetables, fruits, flowers and animal manure.

“Biomass hydrolysate” refers to the product resulting from saccharification of biomass. The biomass may also be pretreated or pre-processed prior to saccharification.

The term “heterologous” means not naturally found in the location of interest. For example, a heterologous gene refers to a gene that is not naturally found in the host organism, but that is introduced into the host organism by gene transfer. For example, a heterologous nucleic acid molecule that is present in a chimeric gene is a nucleic acid molecule that is not naturally found associated with the other segments of the chimeric gene, such as the nucleic acid molecules having the coding region and promoter segments not naturally being associated with each other.

As used herein, an “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe. The term “complementary” is used to describe the relationship between nucleotide bases that are capable of hybridizing to one another. For example, with respect to DNA, adenosine is complementary to thymine and cytosine is complementary to guanine.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations may be performed using the MegAlign program of the LASERGENE bioinformatics computing suite (DNASTAR Inc., Madison, Wis.).

Multiple alignment of the sequences is performed using the “Clustal method of alignment” which encompasses several varieties of the algorithm including the “Clustal V method of alignment” corresponding to the alignment method labeled Clustal V (described by Higgins and Sharp, CABIOS. 5:151-153 (1989); Higgins, D. G. et al., Comput. Appl. Biosci., 8:189-191 (1992)) and found in the MegAlign v8.0 program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191(1992); Thompson, J. D. et al, Nucleic Acid Research, 22 (22): 4673-4680, 1994) and found in the MegAlign v8.0 program of the LASERGENE bioinformatics computing suite (DNASTAR Inc.). Default parameters for multiple alignment (stated as protein/nucleic acid (GAP PENALTY=10/15, GAP LENGTH PENALTY=0.2/6.66, Delay Divergen Seqs (%)=30/30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 50%, 55%, 60%, 65%, 70%, 75%, 80%, 85%, 90%, or 95%, or any integer percentage from 50% to 100% may be useful in identifying polypeptides of interest, such as 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above identities but typically encode a polypeptide having at least 50 amino acids, preferably at least 100 amino acids, and more preferably at least 125 amino acids.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et. al., Short Protocols in Molecular Biology, 5^{th} Ed. Current Protocols, John Wiley and Sons, Inc., N.Y., 2002. Additional methods used here are in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.).

The present invention relates to engineered yeast strains that have xylose isomerase enzyme activity. A challenge for engineering yeast to utilize xylose, which is the second most predominant sugar obtained from cellulosic biomass, is to produce sufficient xylose isomerase activity in the yeast cell. Xylose isomerase catalyzes the conversion of xylose to xylulose, which is the first step in a xylose utilization pathway. Applicants have found that expression of a bacterial xylose isomerase in a yeast cell that expresses the chaperonins GroES and GroEL results in enzymatically active xylose isomerase, while there is little to no activity with expression of the bacterial xylose isomerase in a yeast cell lacking GroES and GroEL. A yeast cell expressing xylose isomerase activity provides a host cell for expression of a complete xylose utilization pathway, thereby enabling the engineering of a yeast cell that can produce a target compound, such as ethanol, butanol, or 1,3-propanediol, using xylose derived from lignocellulosic biomass as a carbon source.

Yeast Host Cells Any yeast cells that either produce a target chemical, or can be engineered to produce a target chemical, may be used as host cells. Examples of such yeasts include, but are not limited to, yeasts of the genera Kluyveromyces, Candida, Pichia, Hansenula, Schizosaccharomyces, Kloeckera, Schwammiomyces, Yarrowia, and Saccharomyces.

Engineering of a yeast cell for expression of xylose isomerase activity as disclosed herein and for production of a target chemical may occur simultaneously or in any order. In one embodiment, yeast cells that produce ethanol may be used as host cells in engineering to produce the present cells. In one embodiment the yeast cells are capable of anaerobic alcoholic fermentation. The yeast cells may naturally produce ethanol, or may be engineered to produce ethanol, or to produce increased yields of ethanol.

In other embodiments yeast cells that are engineered to express a pathway for synthesis of butanol or 1,3-propanediol are host cells, with engineering steps occurring in any order. Engineering of pathways for butanol synthesis (including isobutanol, 1-butanol, and 2-butanol) have been disclosed, for example in U.S. Pat. No. 8,206,970, US 20070292927, US 20090155870, U.S. Pat. No. 7,851,188, and US 20080182308, which are incorporated herein by reference. Engineering of pathways for 1,3-propanediol have been disclosed in U.S. Pat. No. 6,514,733, U.S. Pat. No. 5,686,276, U.S. Pat. No. 7,005,291, U.S. Pat. No. 6,013,494, and U.S. Pat. No. 7,629,151, which are incorporated herein by reference.

For utilization of xylose as a carbon source, a yeast cell is engineered for expression of a complete xylose utilization pathway. Engineering of yeast such as S. cerevisiae for production of ethanol from xylose is described in Matsushika et al. (Appl. Microbiol. Biotechnol. (2009) 84:37-53) and in Kuyper et al. (FEMS Yeast Res. (2005) 5:399-409). In one embodiment, in addition to engineering a yeast cell as disclosed herein to have xylose isomerase activity, the activities of other pathway enzymes are increased in the cell. Typically the activity levels of five pentose pathway enzymes are increased: xylulokinase (XKS1), transaldolase (TAL1), transketolase 1 (TKL1), D-ribulose-5-phosphate 3-epimerase (RPE1), and ribose 5-phosphate ketol-isomerase (RKI1). Any method known to one skilled in the art for increasing expression of a gene may be used. For example, as described herein in Example 5 these activities may be increased by expressing the host coding region for each protein using a highly active promoter. Chimeric genes for expression are constructed and are integrated into the yeast genome. Alternatively, heterologous coding regions for these enzymes may be expressed in the yeast cell to obtain increased enzyme activities. For additional methods for engineering yeast capable of metabolizing xylose see for example US7622284B2, US8058040B2, U.S. Pat. No. 7,943,366 B2, WO2011153516A2, WO2011149353A1, WO2011079388A1, US20100112658A1, US20100028975A1, US20090061502A1, US20070155000A1, WO2006115455A1, US20060216804A1 and US8129171B2.

### GroES and GroEL Polypeptides in Yeast Host Cells

Bacteria, as well as mitochondria and chloroplasts of eukaryotic cells, have a variety of proteins that assist in the folding of other proteins which are called chaperones. Chaperones that are called chaperonins include proteins named GroEL, HSP60, GroES, and HSP10, which are proteins that mediate folding to produce active enzymes. These chaperonins function in interacting pairs to form active complexes, for example GroEL with GroES, and Hsp60 with Hsp10. These complexes mediate the proper folding of certain proteins to convert them into an enzymatically active form. The present yeast cells express an interacting pair of Group I chaperonin polypeptides. No additional chaperonins or other chaperones are needed in the present cells to convert a xylose isomerase polypeptide into an active enzyme.

Any interacting pair of Group I chaperonin polypeptides may be expressed in the present cells. The individual chaperonin polypeptides of the pair may be from the same organism, or from different organisms, as long as together they form a functional complex. The chaperonins are expressed so that they are active in the cytoplasm of the cell. Chaperonins that are expressed in the nucleus of a eukaryotic cell and are transported into the mitochondria or chloroplast may be engineered so that they remain in the cytoplasm. The coding region for the transit signal sequence, which directs transport into the organelle, can be deleted so that the polypeptide remains in the cytoplasm. For example, Hsp60 and Hsp10 with the transit signal sequences removed may be expressed in a yeast cell to provide the present interacting pair of Group I chaperonin polypeptides in the cytoplasm.

In one embodiment the interacting pair of Group I chaperonin polypeptides is derived from a bacteria. A wide variety of bacteria have chaperonins called GroEL and GroES polypeptides. In one embodiment the present yeast cells express bacterial GroEL and GroES polypeptides. Any bacteria-derived pair of GroEL and GroES polypeptides may be expressed in the present yeast cells. In various embodiments the GroEL and GroES proteins are encoded by genes of bacteria of the genera Actinoplanes, Escherichia, Bacillus, Streptomyces, Burkholderia, Citrobacter, Pseudomonas, Photobacterium, Pantoea, Plautia, Vibrio, Yokenella, Bacteroides, Ruminococcus, or Zymomonas. In one embodiment the GroEL and GroES polypeptides are derived from E. coli.

Typically a GroEL polypeptide is paired with its natural partner GroES polypeptide from the same bacterium. Examples of amino acid sequences of GroEL proteins that may be used in the present cells include, but are not limited to, SEQ ID NOs:1, 3, 5, 7, 9, 11, and 13. In various embodiments the GroEL polypeptide in the present cells has at least about 95% amino acid sequence identity to any of SEQ ID NOs: 1, 3, 5, 7, 9, 11, and 13. The GroEL polypeptide may have at least about 95%, 96%, 97%, 98%, or 99% identity to any of SEQ ID NOs: 1, 3, 5, 7, 9, 11, and 13. Because GroEL proteins are well known, and because of the prevalence of genomic sequencing, suitable GroEL polypeptides may be readily identified by one skilled in the art on the basis of sequence similarity using bioinformatics approaches. Typically BLAST (described above) searching of publicly available databases with known GroEL amino acid sequences, such as those provided herein, is used to identify GroEL polypeptides, and their encoding sequences, that may be used in the present strains. In one embodiment the GroEL polypeptide in the present cells has at least about 95% amino acid sequence identity to the amino acid sequence of the E. coli GroEL (SEQ ID NO:1) or to the amino acid sequence of the Actinoplanes missouriensis GroEL polypeptide of SEQ ID NO:3.

Examples of amino acid sequences of GroES polypeptides that may be used in the present cells include, but are not limited to, SEQ ID NOs:15, 17, 19, 21, 23, 25, and 27. In various embodiments the GroES polypeptide in the present cells has at least about 95% amino acid sequence identity to any of SEQ ID NOs: SEQ ID NOs:15, 17, 19, 21, 23, 25, and 27. The GroES polypeptide may have at least about 95%, 96%, 97%, 98%, or 99% identity to any of SEQ ID NOs:15, 17, 19, 21, 23, 25, and 27. Because GroES polypeptides are well known, and because of the prevalence of genomic sequencing, suitable GroES polypeptides may be readily identified by one skilled in the art on the basis of sequence similarity using bioinformatics approaches as described above. In one embodiment the GroES polypeptide in the present cells has at least about 95% amino acid sequence identity to amino acid sequence of the E. coli GroES (SEQ ID NO:15) or to the amino acid sequence of the Actinoplanes missouriensis GroES polypeptide of SEQ ID NO:17.

The coding region for each GroEL and GroES polypeptide is readily obtained from the genome of the bacterial strain in which it is natively expressed, as well known to one skilled in the art. Native nucleotide sequences encoding each of these proteins may be codon optimized for expression in the yeast host cell to be engineered, as is well known to one skilled in the art. For example, codon-optimized coding sequences for expression in yeast for GroEL polypeptides are provided as SEQ ID NOs:2, 4, 6, 8, 10, 12, and 14, and for GroES polypeptides are provided as SEQ ID NOs:16, 18, 20, 22, 24, 26, and 28. The coding regions for GroEL and GroES are heterologous to the yeast cell. Thus heterologous nucleic acid molecules encoding GroEL and GroES polypeptides are introduced into a yeast cell for expression.

Methods for gene expression in yeasts are known in the art (see for example Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). Expression of genes in yeast typically requires a promoter, operably linked to a coding region of interest, and a transcriptional terminator. A number of yeast promoters can be used in constructing expression cassettes for genes encoding GroES and GroEL, including, but not limited to constitutive promoters FBA1, GPD1, ADH1, GPM, TPI1, TDH3, PGK1, Ilv5, and the inducible promoters GAL1, GAL10, and CUP1. Suitable transcription terminators include, but are not limited to FBAt, GPDt, GPMt, ERG10t, GAL1t, CYC1t, ADH1t, TAL1t, TKL1t, ILV5t, and ADHt.

Suitable promoters, transcriptional terminators, and GroEL and GroES coding regions may be cloned into E. coli-yeast shuttle vectors, and transformed into yeast cells. These vectors allow strain propagation in both E. coli and yeast strains.

Typically the vector contains a selectable marker and sequences allowing autonomous replication or chromosomal integration in the desired host. Typically used plasmids in yeast are shuttle vectors pRS423, pRS424, pRS425, and pRS426 (American Type Culture Collection, Rockville, Md.), which contain an E. coli replication origin (e.g., pMB1), a yeast 2μ origin of replication, and a marker for nutritional selection. The selection markers for these four vectors are His3 (vector pRS423), Trp1 (vector pRS424), Leu2 (vector pRS425) and Ura3 (vector pRS426). Additional vectors that may be used include pHR81 (ATCC #87541), pRS313 (ATCC #77142). Construction of expression vectors with chimeric genes encoding GroEL and GroES may be performed by either standard molecular cloning techniques in E. coli or by the gap repair recombination method in yeast.

The gap repair cloning approach takes advantage of the highly efficient homologous recombination in yeast. Typically, a yeast vector DNA is digested (e.g., in its multiple cloning site) to create a “gap” in its sequence. The “gapped” vector and insert DNAs having sequentially overlapping ends (overlapping with each other and with the gapped vector ends, in the desired order of inserts) are then co-transformed into yeast cells which are plated on the medium containing the appropriate compound mixtures that allow complementation of the nutritional selection markers on the plasmids. The presence of correct insert combinations can be confirmed by PCR mapping using plasmid DNA prepared from the selected cells. The plasmid DNA isolated from yeast can then be transformed into an E. coli strain, e.g. TOP10, followed by mini preps and restriction mapping to further verify the plasmid construct. Finally the construct can be verified by sequence analysis.

Like the gap repair technique, integration into the yeast genome also takes advantage of the homologous recombination system in yeast. Typically, a cassette containing a coding region plus control elements (promoter and terminator) and auxotrophic marker is PCR-amplified with a high-fidelity DNA polymerase using primers that hybridize to the cassette and contain 40-70 base pairs of sequence homology to the regions 5′ and 3′ of the genomic area where insertion is desired. The PCR product is then transformed into yeast cells which are plated on medium containing the appropriate compound mixtures that allow selection for the integrated auxotrophic marker. Transformants can be verified either by colony PCR or by direct sequencing of chromosomal DNA.

### Xylose Isomerase Enzyme Activity in Yeast Host Cells

Expression of xylose isomerases in yeast cells has been problematic; some xylose isomerases have been found to have little to no activity when expressed in yeast cells. For example, the xylose isomerase typically expressed to provide a xylose utilization pathway in Zymomonas, that from E. coli, was found to be barely active in S. cerevisiae, producing about 1000-fold lower activity than expected (Sarthy et. al. (1987) Appl. Environ. Microbiol. 53: 1996-2000). A xylose isomerase disclosed in US 20110318801 as providing higher levels of activity in Zymomonas than the E. coli xylose isomerase, that from Actinoplanes missouriensis, is found herein to be inactive in S. cerevisiae.

In the present yeast cell, at least one gene encoding a xylose isomerase polypeptide is introduced together with at least one gene encoding each amino acid sequence of an interacting pair of Group I chaperonin polypeptides, that are described above. Expression of the xylose isomerase in the presence of the Group I chaperonins gives a higher xylose isomerase specific activity as compared with the specific activity of the same xylose isomerase enzyme expressed in the absence of the interacting pair of Group I chaperonin polypeptides.

Any polypeptide having increased xylose isomerase activity in the presence of Group I chaperonins, and belonging to the classification EC 5.3.1, may be expressed in the present yeast cells. In one embodiment the xylose isomerase is derived from a bacteria. In one embodiment the specific activity of the bacterial xylose isomerase is at least 50% of the xylose isomerase specific activity obtained in yeast cells expressing E. coli GroEL and GroESL chaperonins, and E. coli xylose isomerase. The activity may be at least about 50%, 55%, 60%, 65%, 70%, 75%, 80%, 85%, 90%, or 95% of this level.

Xylose isomerases are classified as belonging to Group I or Group II of xylose isomerases. In the present yeast cell a xylose isomerase polypeptide of either Group I or Group II may be introduced. Bacterial Group I and Group II xylose isomerases are described in US 20110318801, which is incorporated herein by reference. Examples of Group I xylose isomerases are disclosed in US 20110318801 as the even numbered sequences starting with SEQ ID NO:2 and ending with SEQ ID NO:130, as well as SEQ ID NOs:131-147 (Table 3 of US 20110318801). Coding regions for the xylose isomerases are the odd numbered sequences starting with SEQ ID NOs:1 and ending with SEQ ID NO:129 (Table 3 of US 20110318801). Examples of Group II xylose isomerases are disclosed in US 20110318801 as SEQ ID NOs:148-306 (Table 4 of US 20110318801). The following are xylose isomerase amino acid sequences with their SEQ ID NOs herein, and in US 20110318801, respectively: Actinoplanes missouriensis (SEQ ID NO:29; 66), E. coli (SEQ ID NO:31; 219), Streptomyces rubininosus (SEQ ID NO:35; 128), Burkholderia phytofirmans (SEQ ID NO:37; 272), Burkholderia phymatum (SEQ ID NO:39; 258), and Photobacterium profundum (SEQ ID NO:47; 177).

Additional examples of xylose isomerases that may be used in the present yeast cell include those from Bacillus subtilis (SEQ ID NO:33), Citrobacter youngae (SEQ ID NO:41), E. blattae (SEQ ID NO:43), Pseudomonas fluorescens (SEQ ID NO:45), Pantoea stewartii (SEQ ID NO:49), Plautia stali symbiont (SEQ ID NO:51), Pseudomonas syringae (SEQ ID NO:53), Vibrio sp. XY-214 (SEQ ID NO:55), and Yokenella regensburgei (SEQ ID NO:57).

Further examples of xylose isomerases that may be used in the present yeast cell include amino acid sequences identified among translated open reading frames of a metagenomic cow rumen database (Matthias Hess, et al. Science 331:463-467 (2011)) by BLAST searching using xylose isomerase sequences from Ruminococcus flavefaciens FD-1 (SEQ ID NO:96) and Ruminococcus champanellensis 18P13 (SEQ ID NO:97). The sequences identified and tested herein (in Example 9) from an uncultured bacterium from cow rumen were named Ru2 (SEQ ID NO:98) and Ru3 (SEQ ID NO:100).

In one embodiment the xylose isomerase is derived from a bacteria of the genera Actinoplanes, Escherichia, Bacillus, Streptomyces, Burkholderia, Citrobacter, Pseudomonas, Photobacterium, Pantoea, Plautia, Vibrio, Yokenella, Bacteroides, Ruminococcus, or Zymomonas.

In various embodiments the xylose isomerase polypeptide in the present cells has at least about 95% amino acid sequence identity to any of the SEQ ID NOs listed above for xylose isomerases: those disclosed in US 20110318801 as the even numbered sequences starting with SEQ ID NO:2 and ending with SEQ ID NO:130, as well as SEQ ID NOs:131-147 (Table 3 of US 20110318801), also SEQ ID NOs:148-306 (Table 4 of US 20110318801), and additionally sequences herein that are SEQ ID NOs:29, 31, 33, 35, 37, 39, 41, 43, 45, 47, 49, 51, 53, 55, and 57. The amino acid sequence has at least about 95%, 96%, 97%, 98%, or 99% sequence identity to any of the SEQ ID NOs for xylose isomerases listed above and those referred to in US 20110318801. Because xylose isomerase proteins are well known, and because of the prevalence of genomic sequencing, suitable xylose isomerase proteins may be readily identified by one skilled in the art on the basis of sequence similarity using bioinformatics approaches. Typically BLAST (described above) searching of publicly available databases with known xylose isomerase amino acid sequences, such as those provided herein, is used to identify xylose isomerase proteins, and their encoding sequences, that may be used in the present strains.

The coding region sequence for each xylose isomerase polypeptide is readily obtained from the genome of the bacterial strain in which it is natively expressed, as is well known to one skilled in the art. Native nucleotide sequences encoding each of these proteins may be codon optimized for expression in the yeast host cell to be engineered, as is well known to one skilled in the art. Examples of coding sequences that are codon optimized for expression in S. cerevisiae, for xylose isomerases of SEQ ID NOs that are odd numbers starting with 29 and ending with 57 are SEQ ID NOs that are even numbers starting with 30 and ending with 58, as well as 59 and 60.

Methods for gene expression in yeasts are as described above for GroEL and GroES, and exemplified in Examples herein. The coding region for a bacterial xylose isomerase is heterologous to the yeast cell. Thus a heterologous nucleic acid molecule encoding a xylose isomerase polypeptide is introduced into a yeast cell for expression.

The present invention provides a method for producing a yeast strain that has xylose isomerase activity following the teachings above. In one embodiment a heterologous nucleic acid molecule encoding a GroEL polypeptide and a heterologous nucleic acid molecule encoding a GroES polypeptide, as well as a heterologous nucleic acid molecule encoding a bacterial xylose isomerase polypeptide, are introduced into a yeast cell. In the yeast cell the GroEL and GroES polypeptides are expressed in the cytosol of the cell, the xylose isomerase polypeptide is converted to an active xylose isomerase enzyme, and the specific activity of the xylose isomerase enzyme is higher as compared with the specific activity of the same xylose isomerase enzyme expressed in the absence of the GroEL and GroES polypeptides.

In one embodiment of the present yeast cell the at least one gene encoding xylose isomerase and the at least one gene encoding each amino acid sequence of an interacting pair of Group I chaperonin polypeptides are derived from the same organism. In one embodiment of the present yeast cell the at least one gene encoding xylose isomerase and the at least one gene encoding each amino acid sequence of an interacting pair of Group I chaperonin polypeptides are derived from different organisms. For example, the coding regions for the Group I chaperonin polypeptides may be derived from E. coli while the coding regions for the xylose isomerase may be derived from Citrobacter youngae, Yokenella refensburgei, or Pseudomonas syringae as in Example 7 herein. In one embodiment the xylose isomerase specific activity in a yeast cell, having the coding regions for the interacting pair of Group I chaperonin polypeptides and the coding region for the xylose isomerase derived from different organisms, is at least 50% of the specific activity in a yeast cell in which the coding regions for the interacting pair of Group I chaperonin polypeptides and the coding region for the xylose isomerase are derived from the same bacterium. The activity may be at least about 50%, 55%, 60%, 65%, 70%, 75%, 80%, 85%, 90%, or 95% of this level.

In one embodiment the present yeast cell has, in combination, the features described above which provide xylose isomerase activity, as well as a complete xylose utilization pathway as described above, and thereby the cell is able to grow on xylose as a sole carbon source. In one embodiment the cell additionally produces a target compound that the cell either naturally synthesizes, or is engineered to synthesize. In various embodiments the target compound is ethanol, butanol, or 1,3-propanediol, pathways for which are referenced above. Thus the present cell is able to utilize xylose in the synthesis of a target compound. Xylose may be the sole carbon source, or it may be one component of the carbon source. Additional carbon source components may include glucose and other components that the cell is naturally able to metabolize, or is engineered to metabolize.

The present yeast cell expresses an active xylose isomerase enzyme when it is grown in a nutrient medium that supports growth of yeast cells. Thus the present invention provides a method for expressing an active bacterial xylose isomerase enzyme in yeast comprising:
a) providing a yeast host cell having at least one gene encoding each amino acid sequence of an interacting pair of Group I chaperonin polypeptides and at least one gene encoding a xylose isomerase polypeptide;
wherein:
i) the interacting pair of Group I chaperonins are active in the cytosol of the cell;
ii) the xylose isomerase polypeptide is converted to an active xylose isomerase enzyme; and
iii) the specific activity of the xylose isomerase enzyme is higher as compared with the specific activity of the same xylose isomerase enzyme expressed in the absence of the interacting pair of Group I chaperonin polypeptides; and
b) growing the yeast cell of a) whereby xylose isomerase polypeptide is converted to an active xylose isomerase enzyme.
   In one embodiment the yeast cell has a complete xylose utilization pathway and is grown in a medium using xylose as a sole carbon source. More typically, the yeast cell is grown in medium containing xylose as well as other sugars such as glucose and arabinose. This allows effective use of the sugars found in a hydrolysate medium that is prepared from cellulosic biomass by pretreatment and saccharification.

In one embodiment the yeast cell has a metabolic pathway that produces a target compound. In one embodiment the target compound is selected from the group consisting of ethanol, butanol, and 1,3-propanediol. Yeast cells having these metabolic pathways are described above.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

General Methods

The meaning of abbreviations is as follows: “kb” means kilobase(s), “bp” means base pairs, “nt” means nucleotide(s), “hr” means hour(s), “min” means minute(s), “sec” means second(s), “d” means day(s), “L” means liter(s), “ml” or “mL” means milliliter(s), “4” means microliter(s), “μg” means microgram(s), “ng” means nanogram(s), “mg” means milligram(s), “mM” means millimolar, “μM” means micromolar, “nm” means nanometer(s), “μmol” means micromole(s), “pmol” means picomole(s), “XI” is xylose isomerase, “nt” means nucleotide.

Standard recombinant DNA and molecular cloning techniques used here are well known in the art and are described by Sambrook, J., Fritsch, E. F. and Maniatis, T., Molecular Cloning: A Laboratory Manual, 2^{nd} ed., Cold Spring Harbor Laboratory: Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987), and by Methods in Yeast Genetics, 2005, Cold Spring Harbor Laboratory Press, Cold Spring Harbor, N.Y.

### Media and Plates

YPD medium: 10 g/L yeast extract, 20 g/L peptone (both from Difco), plus varied glucose concentration

CM+Glucose-Ura plates (Teknova Inc, Hollister, Calif.)

CM+Glucose-His plates (Teknova Inc, Hollister, Calif.)

CM+Glucose-Ura liquid medium: 6.7 g/L yeast nitrogen base without amino acids (Amresco, Solon, Ohio), 0.77 g/L minus ura Drop Out supplement (Clontech Laboratories, Mountain View, Calif.), 20 g/L glucose

CM+Glucose-His liquid medium: 6.7 g/L yeast nitrogen base without amino acids (Amresco, Solon, Ohio), 0.77 g/L minus his Drop Out supplement (Clontech Laboratories, Mountain View, Calif.), 20 g/L glucose

CM+Glucose-Ura-His liquid medium: 6.7 g/L yeast nitrogen base without amino acids (Amresco, Solon, Ohio), 0.77 g/L minus ura/his Drop Out supplement (Clontech Laboratories, Mountain View, Calif.), 20 g/L glucose

### HPLC Analysis

Fermentation samples were taken at timed intervals and analyzed for EtOH, and xylose using either a Waters HPLC system (Alliance system, Waters Corp., Milford, Mass.) or an Agilent 1100 Series LC; conditions=0.6 mL/min of 0.01 N H₂SO₄, injection volume=10 μL, autosampler temperature=10° C., column temperature=65° C., run time=25 min, detection by refractive index (maintained at 40° C.). The HPLC column was purchased from BioRad (Aminex HPX-87H, BioRad Inc., Hercules, Calif.). Analytes were quantified by refractive index detection and compared to known standards.

### Example 1

### AMxyIA, ECgroES, and ECgroEL Expression Cassettes Constructed in Yeast Shuttle Vectors

Vectors were prepared for yeast engineering to study whether the Actinoplanes missouriensis xylose isomerase (AMXI) can be expressed and function in Saccharomyces cerevisiae. AMXI is a group I xylose isomerase, which was found to provide higher activity than other prokaryotic xylose isomerases when expressed in Zymomonas mobilis, as described in US 20110318801. In addition, to study effects of co-expressing the Escherichia coli GroES and GroEL chaperonin coding sequences, ECgroEL and ECgroES, yeast shuttle vectors were constructed for their expression.

The AMxyIA, ECgroES, and ECgroEL genes encode a 394-aa AMXI protein (SEQ ID NO:29), a 548-aa ECGroEL protein (SEQ ID NO:1), and a 97-aa ECGroES protein (SEQ ID NO:15), respectively. The gene sequences are available in Gene Bank with accession numbers of X16042, NC313150, and NC313151, respectively. Coding sequences for the proteins of SEQ ID NOs:29, 1, and 15 were codon-optimized for expression in S. cerevisiae (SEQ ID NOs:30, 2, and 16, respectively) and synthesized de novo in chimeric genes by GenScript Corporation (Piscataway, N.J.). During the synthesis, a 1,184-nt promoter of the S. cerevisiae acetohydroxyacid reductoisomerase gene (ILV5p) with a 5′ NotI site and a 3′ PmeI site was added upstream of the 1,185-nt AmxyIA coding sequence, and a 635-nt terminator of the S. cerevisiae acetohydroxyacid reductoisomerase gene (ILV5t) with a 5′ SfiI site and a 3′ XhoI site was added downstream of the AMxyIA. The resulting synthesized DNA segment formed a 3,036-nt chimeric AMxyIA expression cassette (SEQ ID NO:61).

A chimeric ECgroES expression cassette was synthesized that included a 679-nt promoter of the S. cerevisiae glyceraldehyde-3-phosphate dehydrogenase gene (GPDp) with a 5′ BglII site, a 294-nt codon-optimized coding region of ECgroES and a 252-nt terminator of the S. cerevisiae iso-1-cytochrome C gene (CYC1t) with a 5′ PacI site and a 3′ NotI site. The resulting 1,247-nt chimeric ECgroES expression cassette is SEQ ID NO:62. A chimeric ECgroEL expression cassette was synthesized that included a 678-nt promoter of the S. cerevisiae alcohol dehydrogenase I gene (ADH1p) with a 5′ EcoRI site, a 1,647-nt codon-optimized coding region of ECgroEL, and a 314-nt terminator of the S. cerevisiae alcohol dehydrogenase I gene (ADH1t) with a 5′ PacI site and a 3′ SpeI site. The resulting 2,678-nt chimeric ECgroEL expression cassette is SEQ ID NO:63.

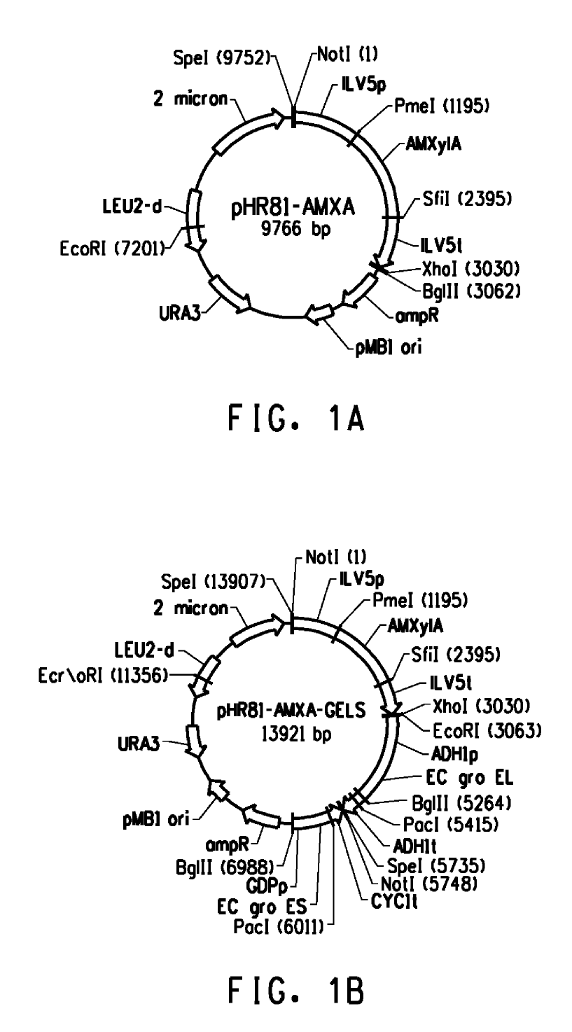

The AMxyIA expression cassette was cloned into a shuttle vector (ATCC #87541), generating a 9,766-bp vector called pHR81-AMXA (SEQ ID NO:64, see FIG. 1A diagram). The pHR81 vector contains a pMB1 origin and an ampicillin resistance (ampR) marker to allow plasmid propagation and selection, respectively, in E. coli. In addition, pHR81 has a 2 micron replication origin, a URA3 selection marker, and LEU 2-d for propagation and selection in yeast, which is correlated with high copy number in S. cerevisiae when grown in medium lacking leucine, Selection for URA3 produces a plasmid copy number of 20 to 40, while selection for LEU2-d produces a plasmid copy number of 100 to 200.

The AmxyIA, ECgroES and ECgroEL expression cassettes were cloned into a pHR81 vector, resulting in a 13,921-bp vector called pHR81-AMXA-GELS (SEQ ID NO:65, see FIG. 1B) In this vector the ECgroEL expression cassette is located downstream of the AMxyIA expression cassette and the ECgroES expression cassette is downstream of the ECgroEL expression cassette, in the opposite orientation.

The ECgroES and ECgroEL expression cassettes were also cloned in a pRS423 vector in opposite orientation, forming a 9,684-bp vector called pRS423-GELS (SEQ ID NO:66, FIG. 1A). Similar to pHR81, the pRS423 shuttle vector (ATCC 77104) provides a pMB1 origin and an ampR marker to allow plasmid propagation in E. coli. It also provides a 2 micron origin for plasmid propagation in S. cerevisiae but uses a HIS3 marker for selection, resulting in about 20 copies in S. cerevisiae.

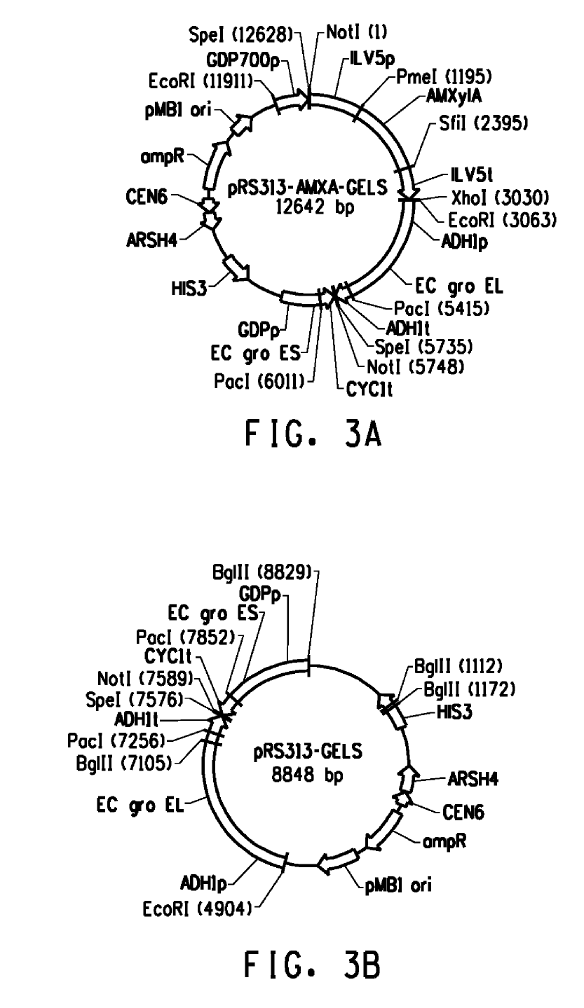

The AMxyIA, ECgroES, and ECgroEL expression cassettes were cloned together in a pRS313 shuttle vector in the same order as in pHR81-AMXA-GELS, forming a 12,642-bp vector called pRS313-AMXA-GELS (SEQ ID NO:67, see FIG. 3A). Also the ECgroES and ECgroEL expression cassettes were cloned into pSR313 in the same order as in pRS423-GELS, producing a vector of 8,848-bp called pRS313-GELS (SEQ ID NO:68, see FIG. 1B). The pRS313 backbone (ATCC #77142) contains a pMB1 origin and an ampR marker for propagation in E. coli. In addition it has a CEN6/ARSH4 origin and HIS3 marker for vector selection and maintenance in S. cerevisiae, resulting in 1 to 2 copies per cell.

### Example 2

### Characterization of A. missourinesis Xylose Isomerase Expression in Yeast Together with E. coli GroES and GroEL Expression

S. cerevisiae strain BY4741 (ATCC 4040002) is a common laboratory strain with a genotype of [MATa his3Δ1 leu2Δ0 met15Δ0 ura3Δ0]. In order to transform it with the constructed yeast shuttle vectors, competent cells of BY4741 were prepared using the Frozen-EZ Yeast Transformation II Kit from Zymo Research (Orange, Calif.). Briefly, 1 mL of overnight grown BY4741 strain was diluted 10 fold using fresh YPD medium and cultured for 4 to 6 hours at 30° C. to reach mid-log phase. Cells were collected by centrifuging at 500×g for 4 minutes, washed with EZ-1 solution, and then resuspended in 1 mL EZ-2 solution. The resulting competent cells could be stored at −80° C. To introduce pHR81-AMXA-GELS and pRS313-AMXA-GELS into BY4741, 50 μL of competent cells were mixed with 1 μg (<5 μL in volume) of vector DNA. Then, 500 μL of EZ-3 solution was added. The mixture was incubated at 30° C. for 1 hour, with vortexing every 15 minutes. The cells transformed with pHR81-AMXA-GELS were spread on CM+Glucose-Ura plates, while the cells transformed with pRS313-AMXA-GELS were spread on CM+Glucose-His plates. After 2 days incubation at 30° C., the transformants grew and became visible. Colonies were streaked to a fresh CM+Glucose-Ura or CM+Glucose-His plate and grown for another 2 days. The resulting transformants containing pHR81-AMXA-GELS were named BY4741 SC8 and those containing pRS313-AMXA-GELS were named BY4741SC9.

For characterization of AMxyIA, ECgroEL, and ECgroES expression, two BY4741SC8 transformants (#1 and #2), two BY4741SC9 transformants (#1 and #2), and the parental BY4741 strain were grown in CM+Glucose-Ura, CM+Glucose-His, and YPD liquid media, respectively at 30° C. overnight. Cell density reached an OD₆₀₀ value of 3. In order to estimate copy numbers of the vectors, 1 μL of each overnight culture was mixed with 46 μl of TE buffer and 1 μl Zymolyase (Zymoresearch. Orange, Calif.), incubated at 37° C. for 30 minutes, and then heated to 95° C. for 10 minutes. The prepared cell lysate samples were subjected to Real Time PCR to estimate plasmid copy number in each transformant, using an Applied Biosystems 7900 Sequence Detection System instrument. The target genes were URA3 for the pHR81 vector, HIS3 for the pRS313 vector, and TEF1 (encodes for Translational elongation factor EF-1 alpha) as an internal control. Wild type S. cerevisiae cell lysate was also prepared to use as a control since it has one copy of genomic URA3 and HIS3. A 20-μL Real Time PCR reaction included the following reagents (2×TaqMan master Mix from ABI-Gene): 10 μl of ABI TaqMan Universal PCR Master Mix w/o UNG, 0.2 μl of 100 μM forward and reverse primers, 0.05 μl of 100 μM TaqMan probe, 1 μl of cell lysate, and 8.55 μL RNase free water. The PCR primers and dual labeled TaqMan probes were designed using Primer Express v2.0 software from Applied Biosystems and were purchased from Sigma-Genosys (Woodlands, Tex.). Primers were qualified for real time quantitation using a dilution series of genomic DNA. A linear regression was performed for each primer and probe set and the efficiencies were confirmed to be within 90-110%. The primer and probe SEQ ID NOs are given in Table 4.

TABLE 4
Primers and probes used in Real Time PCR analysis

| Gene | Primer Name | Direction | SEQ ID NO |
|---|---|---|---|
| HIS3 | HIS3-566F | Fwd | 69 |
| | HIS3-638R | Rev | 70 |
| | HIS3-590T | Probe | 71 |
| URA3 | URA3-512F | Fwd | 72 |
| | URA3-581R | Rev | 73 |
| | URA3-534T | Probe | 74 |
| TEF1 | tef1-739F | Fwd | 75 |
| | tef1-811R | Rev | 76 |
| | tef1-765T | Probe | 77 |

PCR reactions were heated at 95° C. for 10 minutes, followed by 40 cycles of denaturing at 95° C. for 15 seconds and annealing/extending at 60° C. for 1 minute. All reactions were run in triplicate, and the results were averaged. The relative quantitation of the target genes URA3, HIS3, and TEF1 in the lysate samples was calculated using the ΔΔCt method (ABI User Bulletin). The Ct value of the TEF1 gene was used to normalize the quantitation of the URA3 and HIS3 genes for differences in the number of cells added to each reaction. The relative copy number (RCN) is the fold difference in the quantitation of the target genes in a strain relative to that in a wild type strain which has one copy of URA3 and HIS3. Though BY4741 has no URA3 and HIS3, in this experiment BY4741 showed one copy of URA3 and HIS3. Results are shown in Table 5.

TABLE 5
Relative Copy Numbers of URA3 and HIS genes in transformants
containing pHR81-AMXA-GELS (BY4741SC8) and those
containing pRS313-AMXA-GELS (BY4741SC9)

| Strain | RCN of URA3 | RCN of HIS3 |
|---|---|---|
| BY4741 | 0.9 | 1.4 |
| BY4741SC8-1 | 54.3 | 1.8 |
| BY4741SC8-2 | 79.2 | 2.7 |
| BY4741SC9-1 | 5.8 | 6.6 |
| BY4741SC9-2 | 3.2 | 9.0 |

These data show that BY4741 SC8-1 and BY4741 SC8-2 strains propagated a large number of the pHR81-AMXA-GELS vector, but BY4741 SC9-1 and BY4741 SC9-2 strains only had a few copies of the pRS313-AMXA-GELS vector. BY4741, BY4741 SC9-1, and BY4741 SC9-2 strains have no genomic or plasmid-based URA3, but they show a low RCN rather than zero. A similar situation appears for the HIS3 gene in BY4741, BY4741SC8-1, and BY4741SC8-2 strains. These low numbers indicate background in the real time PCR assay.

To measure expression of transcripts, total RNA was isolated from the above overnight cultures using Qiagen RNeasy Mini Kit, following the manufacture's protocol (Valencia, Calif.)). RNA concentration was determined by using Nanodrop ND-1000 (Thermo Fisher Scientific, Wilmington, Del.). Expression of AMxyIA, ECgroEL, and ECgroES transcripts were examined by quantitative Real Time RT-PCR analysis on an Applied Biosystems 7900 Sequence Detection System instrument using a two-step method. Expression of S. cerevisiae TEF1 RNA was examined as an internal control. In order to eliminate residual genomic DNA, 2 μg of total RNA was first treated with DNAse for 15 minutes at room temperature followed by inactivation for 5 min at 75° C. in the presence of 0.1 mM EDTA. cDNA was generated from 1 μg of DNAse treated RNA using the High Capacity cDNA Reverse Transcription Kit from Applied Biosystems according to the manufacturer's recommended protocol. A 20-μL Real Time PCR reaction included 10 μl ABI TaqMan Universal PCR Master Mix w/o UNG, 0.2 μL of 100 μM forward and reverse primers, 0.05 μl of 100 μM TaqMan probe, 2 μl of 1:10 diluted cDNA, and 7.55 μL of RNAse free water. The PCR primers and dual labeled TaqMan probes were designed using Primer Express v2.0 software from Applied Biosystems and were purchased from Sigma-Genosys. Primers were qualified for real time quantitation using a dilution series of genomic DNA and the PCR conditions detailed below. A linear regression was performed for each primer and probe set and the efficiencies were confirmed to be within 90-110%. The primer and probe SEQ ID NOs are given in Table 6.

TABLE 6
Primers and probes used in Real Time PCR analysis

| Gene | Primer Name | Direction | SEQ ID NO |
|---|---|---|---|
| groEL | groEL-380F | Fwd | 78 |
| | groEL-459R | Rev | 79 |
| | groEL-408T | Probe | 80 |
| groES | groES-200F | Fwd | 81 |
| | groES-277R | Rev | 82 |
| | groES-224T | Probe | 83 |
| xylA | xylA-59F | Fwd | 84 |
| | xylA-128R | Rev | 85 |
| | xylA-81T | Probe | 86 |
| TEF1 | tef1-739F | Fwd | 75 |
| | tef1-811R | Rev | 76 |
| | tef1-765T | Probe | 77 |

PCR reactions were heated at 95° C. for 10 minutes, followed by 40 cycles of denaturing at 95° C. for 15 seconds and annealing/extending at 60° C. for 1 minute. All reactions were run in triplicate and the results averaged. The relative quantitation of the AMxyIA, ECgroEL, ECgroES and S. cerevisiae TEF1 transcripts in the RNA samples was calculated using the ΔΔCt method (ABI). The Ct value of TEF1 RNA was used to normalize the quantitation of the target transcripts for differences in the amount of total RNA added to each reaction. The relative quantitation (RQ) value is the fold difference in expression of the target transcripts in a strain relative to that in the BY4741SC9-1 strain. The results in Table 7 show that, relative to BY4741 SC9-1, its sibling strain BY4741 SC9-2 that only contains a few copies of pRS313-AMXA-GELS expressed similar amounts of the target transcripts; BY4741 SC8-1 and BY4741 SC8-2 strains that contain more copies of the pHR81-AMXA-GELS vector expressed much more of the target transcripts, especially ECgroEL and EcgroES transcripts. The BY4741 control that contains no vector did not express any of the target transcripts.

TABLE 7
Relative Quantitation of transcripts in transformants
containing pHR81-AMXA-GELS (BY4741SC8) and those
containing pRS313-AMXA-GELS (BY4741SC9)

| Strain | RQ of AMxylA | RQ of ECgroEL | RQ of ECgroES |
|---|---|---|---|
| BY4741 | 0 | 0 | 0 |
| BY4741SC8-1 | 2.5 | 16.2 | 18.3 |
| BY4741SC8-2 | 2.6 | 17.2 | 14.4 |
| BY4741SC9-1 | 1.0 | 1.0 | 1.0 |
| BY4741SC9-2 | 0.5 | 2.3 | 3.2 |

In order to make protein extracts, cells from the above overnight cultures were collected by centrifugation and resuspended in Cell Breaking Buffer (CBB) which contains 10 mM TEA (pH 7.5), 10 mM MgSO₄, 10 mM MnCl₂, 1 mM DTT, and Roche cOmplete Mini EDTA-free protease inhibitor cocktail (Indianapolis, Ind.) in an amount of 1 tablet per 50 mL CBB. One milliliter of the cell re-suspension was added into a 2-mL screw-cap bead beating tube containing approximately 1 g of VWR 400 micron acid washed silica beads, and subjected to breakage on a Minibeadbeater (BioSpec products, Bartlesville, Okla.) using 3×1 minute cycles with chilling of the tubes on ice between cycles. The tubes were then centrifuged for 1 min at 15,000×g to pellet large particles and reduce foaming, and 600 μL of supernatant was transferred to a new microcentrifuge tube and centrifuged at 15,000×g for one hour at 4° C. Finally, 500 μL of the supernatant was transferred to a new microcentrifuge tube and stored as protein extract.

Total protein concentration in the protein extracts were determined in triplicate on a microtiter plate, using Thermo Scientific Coomassie protein assay reagent (Rockford, Ill.) and following the manufacturer's instruction. BSA was used as protein standard. Xylose isomerase (XI) activities in the protein extracts were measured on a Varian Cary 300 Bio spectrophotometer (Agilent Technologies, Santa Clara, Calif.) at 30° C. At first, 0.8 mL of XI assay stock solution (10 mM TEA, pH 7.5, 10 mM MgSO₄ heptahydrate, 10 mM MnCl₂, 0.28 mM NADH, 1 μl/mL sorbitol dehydrogenase) was added to a quartz cuvette and placed on the cuvette holder of the instrument to allow temperature equilibration for 10 minutes. Then, 0.1 mL of the diluted protein extract was added and A₃₄₀ₙₘ was monitored until a stable linear reading was reached. Finally, 0.1 mL of 0.5 M xylose was added to start the reaction. Monitoring at A₃₄₀ₙₘ resulted in a slope Of A₃₄₀ₙₘ change (dA₃₄₀/min), which was used to calculate XI activity. One unit of XI activity was defined as the formation of 1 μmole of D-xylulose per minute at 30° C. It was calculated in equations as follows: U (μmole/min)=slope (dA₃₄₀/min)*volume of reaction (μL)/6220/1 cm; Specific activity (μmole/min-mg)=μmole/min/protein concentration (mg) (US Patent Application 20080081358).

The results given in Table 8 demonstrate that both BY4741SC8 and BY4741SC9 strains had XI activity, but the BY4741 strain did not. Specific activity in the BY4741 SC8 strains was at least about 4-fold higher than that in the BY4741 SC9 strains, indicating that higher copy number of the pHR81-AMXA-GELS vector in BY4741SC8 strains supported a higher level of expression of AMXI activity.

TABLE 8
Xylose isomerase activity in transformants containing
pHR81-AMXA-GELS (BY4741SC8) and those containing
pRS313-AMXA-GELS (BY4741SC9)

| Strain | Specific XI Activity (μmole/min/mg) |
|---|---|
| BY4741 | No activity |
| BY4741SC8-1 | 0.273 |
| BY4741SC8-2 | 0.367 |
| BY4741SC9-1 | 0.048 |
| BY4741SC9-2 | 0.069 |

### Example 3

### Expression of A. missourinesis Xylose Isomerase in Yeast Alone or with E. coli GroES and GroEL

To determine whether the A. missouriensis xylose isomerase alone can be expressed as an active enzyme in yeast, or it requires E. coli chaperonins GroEL and GroES, the pHR81-AMXA vector was transformed into competent cells of the S. cerevisiae BY4741 strain as described in the Example 2. Transformants were selected on a CM+Glucose-Ura plate and recovered strains were named BY4741 SC5.

In addition, a 5-μL DNA mixture containing 1 μg pHR81-AMXA and 1 μg pRS423-GELS, and another 5-μL DNA mixture containing 1 μg pHR81-AMXA and 1 μg pRS313-GELS were each used to transform 50 μL of competent cells of the S. cerevisiae BY4741 strain. The transformants were selected on Teknova CM+Glucose-Ura-His plates. Resulting strains having pHR81-AMXA and pRS423-GELS were named BY4741SC6 while those containing pHR81-AMXA and pRS313-GELS were named BY4741SC7.

For characterization of AMxyIA, ECgroEL, and ECgroES expression in these strains, two BY4741SC6 transformants (#1 and #2) and two BY4741SC7 transformants (#1 and #2) were grown in CM+Glucose-Ura-His liquid medium at 30° C. for overnight, respectively. Two BY4741 SC5 transformants (#1 and #2) were grown in CM+Glucose-Ura liquid medium at 30° C. for overnight. Cell density reached an OD₆₀₀ value of 3.

In order to estimate relative copy number of the transformed vectors in these strains, the cell lysate was prepared, real time PCR was performed, and RCN was calculated as described in Example 2. The target genes were URA3 for the pHR81-AMXA vectors, HIS3 for pRS313-GELS and pRS423-GELS vectors, and TEF1 as an internal control. Wild type S. cerevisiae DNA was used as a standard for containing one copy of URA3 and HIS3. The RCN is the fold difference in the quantitation of the target genes in a strain relative to that in the wild type strain. Results shown in Table 9 confirm that all tested strains contained a large number of pHR81-AMXA vectors.

TABLE 9
Relative Copy Numbers of URA3 and HIS genes in transformants
containing no chaperonins (BY4741SC5), those containing
pHR81-AMXA and pRS423-GELS (BY4741SC6), and those containing
pHR81-AMXA and pRS313-GELS (BY4741SC7)

| Strain | RCN of URA3 | RCN of HIS3 |
|---|---|---|
| BY4741SC5-1 | 58.2 | 2.5 |
| BY4741SC5-2 | 50.7 | 2.5 |
| BY4741SC6-1 | 169.4 | 27.7 |
| BY4741SC6-2 | 157.4 | 41.7 |
| BY4741SC7-1 | 83.6 | 10.4 |
| BY4741SC7-2 | 88.3 | 11.7 |

The results showed that the BY4741SC6 strains contained almost 3 to 4 fold more copies of pRS423-GELS vector than the BY4741 SC7 strains contained of the pRS313-GELS vector (HIS3 assay). Since BY4741 SC5 strains did not receive either of the pRS313-GELS and pRS423-GELS vectors, the 2.5 RCN of HIS3 for these strains represents background in the real time RT-PCR assay. Copies of the pHR81-AMXA vector showed variation among the strains, potentially with some influence of the presence of a second vector.

In order to determine the expression levels of AMxyIA, ECgroEL, and ECgroES transcripts in these strains, total RNA was isolated, quantitative real time RT-PCR analysis was performed, and relative quantitation of transcripts was calculated as described in Example 2. Expression of S. cerevisiae TEF1 RNA was examined as an internal control. The RQ value is the fold difference in expression of the target transcript in a strain relative to that in the BY4741SC9-1 strain, which was shown in the previous example. The results given in Table 10 indicate that all strains express AMxyIA transcripts, though at different levels which correlate in relative level with the vector copy numbers in Table 9. The BY4741SC5 strains express no ECgroEL and ECgroES transcripts due to absence of these genes. The BY4741SC7 and BY4741SC6 strains expressed ECgroEL and ECgroES transcripts, again with relative levels correlating in relative level with the vector copy numbers in Table 9.

TABLE 10
Relative Quantitation of transcripts in transformants
containing no chaperonins (BY4741SC5), those containing
pHR81-AMXA and pRS423-GELS (BY4741SC6), and those containing
pHR81-AMXA and pRS313-GELS (BY4741SC7)

| Strain | RQ of AMxylA | RQ of ECgroEL | RQ of ECgroES |
|---|---|---|---|
| BY4741SC9-1 | 1.0 | 1.0 | 1.0 |
| BY4741SC5-1 | 1.3 | 0 | 0 |
| BY4741SC5-2 | 2.1 | 0 | 0 |
| BY4741SC6-1 | 11.2 | 10.6 | 11.0 |
| BY4741SC6-2 | 9.1 | 12.3 | 9.1 |
| BY4741SC7-1 | 2.9 | 6.8 | 2.3 |
| BY4741SC7-2 | 2.5 | 7.7 | 2.2 |

In order to measure xylose isomerase activity in these strains, protein extracts were prepared and XI activities were measured and calculated as described in Example 2. The results in Table 11 show that the BY4741 SC5 strains did not have XI activity indicating that though the AMxyIA transcript was present, enzymatically active protein was not produced. The BY4741SC7 and BY4741SC6 strains contained levels of XI activity correlating with the relative levels of transcripts and vector copy numbers. Thus expression of ECgroEL and ECgroES in either the pRS423-GELS or pRS313-GELS vector enabled functional expression of AMxyIA. Specific activity of AMXI in BY4741SC6 strains was significantly higher than that in BY4741SC7 strains as shown in Table 11.

TABLE 11
Xylose isomerase activity in transformants containing
no chaperonins (BY4741SC5), those containing pHR81-
AMXA and pRS423-GELS (BY4741SC6), and those containing
pHR81-AMXA and pRS313-GELS (BY4741SC7)

| | Specific XI Activity |
|---|---|
| Strain | (μmole/min/mg) |
| BY4741SC5-1 | No activity |
| BY4741SC5-2 | 0.020 |
| BY4741SC6-1 | 0.477 |
| BY4741SC6-2 | 0.513 |
| BY4741SC7-1 | 0.179 |
| BY4741SC7-2 | 0.355 |

### Example 4

### Expression of Additional Procaryotic Xylose Isomerases in S. cerevisiae with and without Co-Expression of GroEL/GroES

### Construction of Additional Procaryotic Xylose Isomerase Expression Plasmids

To test whether other bacterial xylose isomerases also require GroEL and GroES for functional expression in S. cerevisiae, three other proteins were evaluated using the same fungal host strain and the same expression plasmid that was used for the A. missouriensis xylose isomerase (AMXI). Enzymes tested were from both gram negative (E. coli) and gram positive (Bacillus subtilis and Streptomyces rubiginosus) bacteria. The amino acid sequences that were used for the three test proteins were based on the published amino acid sequences for E. coli xylose isomerase (ECXI), B. subtilis xylose isomerase (BSXI), and S. rubiginosus xylose isomerase (SRXI), which have Gen Bank accession numbers AAB18542 (SEQ ID NO:31), AFQ57693.1 (SEQ ID NO:33), and AAA26838.1 (SEQ ID NO:35), respectively. As was the case for AMXI, the nucleotide sequences for their open reading frames were codon-optimized for expression in S. cerevisiae and synthesized by GenScript Corporation (Piscataway, N.J.). All three synthetic DNA fragments were prepared with a PmeI site just upstream of the start codon and a unique SfiI site immediately following the stop codon. The two restriction sites were used for cloning purposes as described below. The codon-optimized nucleotide sequences for the ECXI, BSXI, and SRXI synthetic DNA fragments are given as SEQ ID NO:32, SEQ ID NO:34 and SEQ ID NO:36, respectively. Table 12 shows the similarity of these proteins to each other and to AMXI at the amino acid sequence level (% Identity). Note that the two most closely related proteins are only 67% identical.

TABLE 12
Xylose Isomerases amino acid sequence % identity

| | | | |
|---|---|---|---|
| AMXI | ECXI | BSXI | SRXI |

| | | | |
|---|---|---|---|
| AMXI | 20 | 22 | 68 |
| ECXI | | 51 | 23 |
| BSXI | | | 23 |

As described in Example 1, plasmid pHR81-AMXA (SEQ ID NO:64, FIG. 1A) is a high-copy number expression plasmid for AMXI. The 5′-end of the codon-optimized AMXA open reading frame is attached to the ILV5 promoter (ILV5p) and its 3′-end is attached to the ILV5 transcriptional terminator (ILV5t). The entire open reading frame is conveniently located between a unique PmeI site that is just upstream from the start codon and a unique SfiI site that is immediately after the stop codon. To generate corresponding expression plasmids for ECXI, BSXI, SRXI, plasmid pHR81-AMXA was digested with PmeI and SfiI and the large vector fragment was purified by agarose gel electrophoresis. The purified vector fragment was then ligated to each of the three synthetic, codon-optimized bacterial xylose isomerase DNA fragments described above after they too were digested with PmeI and SfiI. The resulting xylose isomerase expression plasmids were called pHR81 ilv5p xyIA (ECXI), pHR81 ilv5p xyIA (BSXI), and pHR81 ilv5p xyIA (SRXI).

Introduction of Xylose Isomerase and GroEL/GroES Plasmids into S. cerevisia

Competent BY4741 (ATCC 4040002) cells were prepared using the Frozen-EZ Yeast Transformation II Kit from Zymo Research (Orange, Calif.) and the vendor's protocol as described above. To generate strains that only express AMXI, ECXI, BSX, or SRX1, without co-expression of GroEL/GroES, 50 μL of ice-thawed BY4741 competent cells was mixed with 1 μg plasmid DNA (either pHR81-AMXA, pHR81 ilv5p xyIA (ECXI), pHR81 ilv5p xyIA (BSXI), or pHR81 ilv5p xyIA (SRXI)), and 500 μL of EZ-3 solution was added. After a 1-hr incubation period at 30° C. with shaking at 220 rpm, the mixtures were spread onto CM+Glucose-Ura plates, and the plates were incubated for two days at 30° C. until colonies appeared. Two colonies from each transformation reaction were randomly selected for further characterization, and were patched onto a fresh CM+Glucose-Ura plate. The resulting strains were named Am-A and Am-B for the AMXI strains; Ec-A and Ec-B for the ECXI strains; Bs-A and Bs-B for the BSXI strains; SR-A and SR-B for the SR-XI strains.

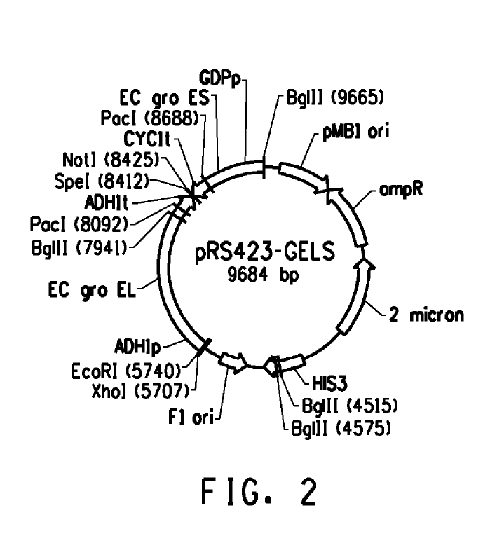

To generate an analogous series of strains that co-express the E. coli GroEL and GroES chaperonins in addition to the above bacterial xylose isomerases, we used the GroEL/GroES expression plasmid pRS423-GELS (SEQ ID NO:9, FIG. 2) that is described in detail in Example I. The transformation protocol was the same as that described above, but 1 μg of pRS423-GELS and 1 μg of xylose isomerase expression plasmid DNA was added to the competent cells, and transformants were plated onto CM+Glucose-Ura-His plates to select for both of the plasmids. The plates were incubated for 2 days at 30° C. until colonies appeared. Two colonies from each transformation reaction were randomly selected for further characterization, and they were patched onto a fresh CM+Glucose-Ura-His plate. These strains were named Am/GroEL/ES-A and —B; Ec/GroEL/ES-A and —B; Bs/GroEL/ES-A and —B; SR/GroEL/ES-A and -B.

### Preparation of Cell-Free Extracts and Protocol for Measuring Xylose Isomerase Activity

The eight strains that only had a xylose isomerase expression plasmid were grown overnight at 30° C. in CM+Glucose-Ura liquid medium to an OD600 value of 3.0-4.7. Thirty milliliter aliquots of the cultures were then harvested by centrifugation, and the drained cell pellets were rapidly frozen on dry ice and stored at −80° C. The same procedure was used for the eight strains that also had the GroEL/GroES expression plasmid but the growth medium was CM+Glucose-His-Ura.

Cell breaking buffer was prepared with 10 mM TEA, pH 7.5, 10 mM MgSO₄, 10 mM MnCl₂, 1 mM DTT, and one tablet of cOmplete Mini, EDTA-free protease inhibitor cocktail (Roche Diagnostics GmbH, Mannheim, Germany) in 50 mL total volume. Bead beating tubes were prepared with approximately 1 gram of 400 micron acid washed silica beads (VWR) in a 2-mL screw cap tube. Cell pellets were resuspended to a concentration of 100 OD units/mL of breaking buffer and 1 mL of this suspension was added to the bead beating tube. Tubes were stored on ice. Cell breakage was performed using a Minibeadbeater (Biospec Products; Bartlesville, Okla.) using 3×1 minute cycles with chilling of the tubes on ice between cycles. Tubes were centrifuged for 1 min at 15,000×g to pellet large particles and reduce foaming. A 600-μl aliquot was removed and transferred to a new microcentrifuge tube. These were centrifuged at 15,000×g for one hour at 4° C. A 500-μl aliquot of the supernatant was transferred to a new microcentrifuge tube and stored on ice. The samples were then diluted 15-fold by adding 20 μl of the extract to 280 μl of breaking buffer. The remaining extract was frozen on dry ice and transferred to the −80° C. freezer while the dilutions were stored on ice until analysis, which was carried out on the same day.

Xylose isomerase enzyme activity was measured spectrophotometrically by monitoring NADH disappearance at 340 nm, using a coupled enzyme assay with sorbitol dehydrogenase. A stock xylose isomerase assay solution was prepared by adding the volumes found in Table 13 to a tube that was stored at room temperature. A solution of 0.5 M xylose was also prepared and stored in a separate tube. All chemicals were obtained from Sigma Aldrich and the source of sorbitol dehydrogenase was sheep liver.

TABLE 13
Assay stock solution composition and final
assay concentration of components

| | volume for | | final concentration | |
|---|---|---|---|---|
| Reagent | stock solution | | in assay | |
| 1M TEA, pH 7.5 | 200 | μL | 10 | mM |
| 1M MgSO4 heptahydrate | 200 | μL | 10 | mM |
| 1M MnCl2 | 200 | μL | 10 | mM |
| NADH (1 mg/mL in water) | 4 | mL | 0.28 | mM |
| Sorbitol Dehydrogenase | 2 | mL | 1 | U/mL |
| (10 U/mL in water) | | | | |

| | | | |
|---|---|---|---|
| Water | 9.4 | mL | 1 mL assay |
| | | | volume |

A Cary 300 Bio spectrophotometer (Varian, Inc. purchased by Agilent Technologies, Santa Clara, Calif.) was set-up for a 10 minute assay time and the cuvette block heater was set to 30° C. Eight hundred microliters of the assay stock solution was added to a quartz cuvette and inserted into the instrument cuvette holder and the temperature was allowed to equilibrate for 10 minutes. One hundred microliters of the extraction dilution was added to the cuvette and monitoring at A₃₄₀ₙₘ was initiated. This was continued until a stable linear signal was obtained (background) which typically took 2-4 minutes. Next, 100 μl of 0.5 M xylose was added to start the reaction. Monitoring at A₃₄₀ₙₘ continued until a stable linear signal was obtained (signal) which typically took 2-4 minutes. Then the resulting change in slope at A₃₄₀ₙₘ was used to calculate XI activity. One unit of enzyme activity was defined as the formation of 1 μmole of D-xylulose per minute at 30° C. It was calculated in equations as follows: U (μmole/min)=slope (dA₃₄₀/min)*volume of reaction (μL)/6220/1 cm; Specific activity (μmole/min-mg)=μmole/min/protein concentration (mg) (US Patent Application 20080081358).

A protein assay was performed on the same dilutions used for the xylose isomerase activity assay. A 15-μl aliquot of each sample was added to a microtiter plate in triplicate. Standard BSA protein standards (ThermoFisher Scientific) were also added in triplicate. Then 300 μl of Coomassie Plus—The Better Bradford Assay Reagent (ThermoFisher Scientific) was added and the plate was equilibrated at room temperature for 15 minutes. The A₅₉₅ₙₘ was obtained. A trend line with a polynomial fit was used for the standards to calculate the protein concentration for the samples.

As already noted, the four bacterial xylose isomerases that were used in the present work were chosen because previous attempts in other laboratories to express them in S. cerevisiae did not result in significant amounts of catalytically active enzymes. Indeed, the results shown in Table 14 validate these earlier observations: none of the four proteins produced detectable amounts of enzyme activity when they were expressed in S. cerevisiae in the absence of E. coli GroEL and GroES. In marked contrast, all of the proteins yielded active enzymes when they were co-expressed with GroEL/GroES in the same fungal host. The highest enzyme activity was obtained with the E. coli homolog (ECXI), which had a specific activity of >0.5 U/mg protein. However, the three other test proteins also resulted in reasonable amounts of catalytically active enzyme based on literature values for other xylose isomerases that do not require GroEL and GroES for functional expression in S. cerevisiae. The above experiments provide a dramatic demonstration of the beneficial effects of bacterial molecular chaperones on the functional expression of prokaryotic xylose isomerases that would otherwise fail to fold properly in yeast cytosol.

TABLE 14
Bacillus subtilis and Streptomyces rubiginosus
xylose isomerase activity assay results

| | | | Specific Activity |
|---|---|---|---|
| strain | Xylose isomerase | GroEL/ES | (μmole/min/mg) |
| AM - A | A. missouriensis | No | 0.000 |
| AM - B | A. missouriensis | No | 0.002 |
| Ec - A | E. coli | No | 0.000 |
| Ec - B | E. coli | No | 0.003 |
| Bs-A | B. subtilis | No | 0.002 |
| Bs-B | B. subtilis | No | 0.004 |
| SR-A | S. rubiginosus | No | 0.000 |
| SR-B | S. rubiginosus | No | 0.000 |
| Am/GroEL/ES-A | A. missouriensis | Yes | 0.396 |
| Am/GroEL/ES-B | A. missouriensis | Yes | 0.378 |
| Ec/GroEL/ES-A | E. coli | Yes | 0.521 |
| Ec/GroEL/ES-B | E. coli | Yes | 0.542 |
| Bs/GroEL/ES-A | B. subtilis | Yes | 0.160 |
| Bs/GroEL/ES-B | B. subtilis | Yes | 0.176 |
| SR/GroEL/ES-A | S. rubiginosus | Yes | 0.185 |
| SR/GroEL/ES-B | S. rubiginosus | Yes | 0.185 |

### Example 5

### Up-Regulation of the Native Pentose Pathway in S. cerevisiae

In addition to expression of an active xylose isomerase enzyme, a robust pentose pathway is necessary for efficient use of xylose and ethanol production under oxygen-limiting conditions in S. cerevisiae. The pentose pathway consists of five enzymes. In S. cerevisiae, these proteins are xylulokinase (XKS1), transaldolase (TAL1), transketolase 1 (TKL1), D-ribulose-5-phosphate 3-epimerase (RPE1), and ribose 5-phosphate ketol-isomerase (RKI1). In order to increase the expression of these proteins, their coding regions from the S. cerevisiae genome were cloned for expression under different promoters and integrated in the S. cerevisiae chromosome. The GRE3 locus encoding aldose reductase was chosen for integration. To construct such this strain, the first step was the construction of an integration vector called P5 Integration Vector in GRE3.

The sequence of the P5 Integration Vector in GRE3 is given as SEQ ID NO:87, and the following numbers refer to nucleotide positions in this vector sequence. Gaps between the given nt numbers include sequence regions containing restriction sites. The TAL1 coding region (15210 to 16217) was expressed with the TPI1 promoter (14615 to 15197) and uses the TAL1t terminator. The RPE1 (13893 to 14609) coding region was expressed with the FBA1 promoter (13290 to 13879) and uses the terminator at the upstream end of the TPI1 promoter. RKI1 coding region (nt 11907 to 12680) was expressed with the TDH3 promoter (11229 to 11900) and uses the GPDt (previously called TDH3t) terminator. The TKL1 coding region (nt 8830 to 10872) was expressed with the PGK1 promoter (nt 8018 to 8817) and uses the TKL1t terminator. The XKS1 coding region (nt 7297 to 5495 to) was expressed with the Ilv5 promoter (nt 8009 to 7310) and uses the ADH terminator. In this integration vector, the URA3 marker (nt 332 to 1135) was flanked by loxP sites (nt 42 to 75 and nt 1513 to 1546) for recycling of the marker. The vector contains integration arms for the GRE3 locus (nt 1549 to 2089 and nt 4566 to 5137). This P5 Integration Vector in GRE3 can be linearized by digesting with the KasI enzyme before integration.

The yeast strain chosen for this study was BP1548 which is a haploid strain derived from prototrophic diploid strain CBS 8272 (Centraalbureau voor Schimmelcultures (CBS) Fungal Biodiversity Centre, Netherlands). This strain is in the CEN.PK lineage of Saccharomyces cerevisiae strains. BP1548 contains the MATa mating type and deletions of the URA3 and HIS3 genes.

To produce BP1548, first CBS 8272 was sporulated and a tetrad was dissected to yield four haploid strains using standard procedures (Amberg et al., Methods in Yeast Genetics, 2005). One of the MATa haploids, PNY0899, was selected for further modifications. The URA3 coding sequence (ATG through stop codon) and 130 bp of sequence upstream of the URA3 coding sequence was deleted by homologous recombination using a KanMX deletion cassette flanked by loxP sites, primer binding sites, and homologous sequences outside of the URA3 region to be deleted. After removal of the KanMX marker using the cre recombinase, a 95 bp sequence consisting of a loxP site flanked by the primer binding sites remained as a URA3 deletion scar in the genome (SEQ ID NO:88). This sequence is located in the genome between URA3 upstream sequence (SEQ ID NO:89) and URA3 downstream sequence (SEQ ID NO:90). The HIS3 coding sequence (ATG up to the stop codon) was deleted by homologous recombination using a scarless method. The deletion joins genomic sequences that were originally upstream (SEQ ID NO:91) and downstream (SEQ ID NO:92) of the HIS3 coding sequence. The KasI integration fragment containing all five pentose pathway genes in vector P5 Integration Vector in GRE3 was transformed into the BP1548 strain using the Frozen-EZ Yeast Transformation II Kit from Zymo Research (Irvine, Calif.). Transformants were selected on synthetic dropout (SD) medium lacking uracil. To recycle the URA3 marker, the CRE recombinase vector pJT254 (SEQ ID NO:93) was transformed into these integrated strains. This vector was derived from pRS413 and the cre coding region (nt 2562 to 3593) was under the control of the GAL1 promoter (nt 2119 to 2561). Strains that could no longer grow on SD (-uracil) medium were selected. Further passages on YPD medium was used to cure the plasmid pJT257. The resulting strain was designated as C52-79.

### Example 6

### Growth and Ethanol Production by S. cerevisiae Containing Different Bacterial Xylose Isomerases and E. coli Chaperonins

The constructed C52-79 S. cerevisiae strain could not use xylose as an energy and carbon source since it lacks xylose isomerase activity. In this experiment, xyIA chimeric genes encoding xylose isomerases from bacterial sources were expressed in the C52-79 host with or without the presence of E. coli chaperonins. The bacterial xylose isomerases tested are those from Actinoplanes missouriensis (AMyXI; SEQ ID NO:29; YP_{—}005460771), Burkholderia phytofirmans PsJN (BPP; SEQ ID NO:37; YP_{—}001890302), Burkholderia phymatum (BPS; SEQ ID NO:39; YP_{—}001858563), Citrobacter youngae (CYXI; SEQ ID NO:41; ZP_{—}06571492), Escherichia blattae (EBXI; SEQ ID NO:43; YP_{—}006317764), E. coli MG1655 (ECXI; SEQ ID NO:31; NP_{—}418022), Pseudomonas fluorescens (PFSXI; SEQ ID NO:45; EIK60355), Photobacterium profundum (PPXI; SEQ ID NO:47; YP_{—}128690), Pantoea stewartii (PS; SEQ ID NO:49; ZP_{—}09830211), Plautia stali symbiont (PSS; SEQ ID NO:51; ZP_{—}0825515), Pseudomonas syringae (PST; SEQ ID NO:53; (ZP_{—}03398764), Vibrio sp. XY-214 (VSXI; SEQ ID NO:55; BAI23199), and Yokenella regensburgei (YRXI; SEQ ID NO:57; ZP_{—}09387709): (abbreviation; SEQ ID NO; Accession number). The coding sequence for each of these proteins was codon-optimized for expression in S. cerevisiae (SEQ ID NOs:59, 38, 40, 42, 44, 60, 46, 48, 50, 52, 54, 56, and 58, respectively) and synthesized de novo in chimeric genes by GenScript Corporation (Piscataway, N.J.). The ILV5p promoter and IVL5t terminator were used in each chimeric gene, which was cloned into the pHR81 vector as described in Example 1. The resulting plasmids were called pHR81 ilv5p xyIA (AMyXI), HR81 ilv5p xyIA (BPPXI), pHR81 ilv5p xyIA (BPSXI), pHR81 ilv5p xyIA (CYXI), pHR81 ilv5p xyIA (EBXI), pHR81 ilv5p xyIA (ECXI), pHR81 ilv5p xyIA (PFSXI), pHR81 ilv5p xyIA (PPXI), pHR81 ilv5p xyIA (PSXI), pHR81 ilv5p xyIA (PSSXI), pHR81 ilv5p xyIA (PSTXI), pHR81 ilv5p xyIA (VSXI), and pHR81 ilv5p xyIA (YRXI), The plasmid pHR81 ilv5p xyIA (ECXI) is the same construction as previously made in Example 4 named pHR81-ECXA. The plasmid pHR81 ilv5p xyIA (AMyXI) uses different codon optimization than the previously constructed pHR81-AMXA (Example 1) that was used for A. missouriensis XI expression in Examples 1 and 2.

The same plasmid pHR81-AMXA-GELS that was described in Example 1 was used for expression of E. coli GroES and GroEL. Each xylose isomerase expression plasmid was co-transformed with the groES and groE expression plasmid pRS423-GELS, into the C52-79 strain (Example 5) and transformants were selected as described in Example 4. The yeast strains obtained as described above expressing xyIA genes in the presence of E. coli groES and groEL were tested in YPX medium (10 g/l yeast extract, 20 g/l peptone, and 40 g/l of xylose). To perform this test, strains were inoculated into 10 ml of YPX medium in 50 ml tissue culture tubes at a starting OD of 0.5 at 600 nm. The lids were tightly closed and the tubes were placed in a 30° C. rotary shaker set at speed of 225 rpm. After 24, 48 or 72 hours, samples were taken to measure the xylose and ethanol concentrations by HPLC as in General Methods. Three strains from each transformation were tested and results shown in Table 1 were the average and standard deviation for each set.

As shown in Table 15, with the expression of E. coli chaperones, all strains tested enabled the consumption of xylose and at the same time, ethanol production. Strains expressing xylose isomerases from C. youngae and E. blattae show the best performance. In the absence of E. coli chaperonins, no xylose consumption or ethanol production was observed.

TABLE 15
Growth rate, xylose consumption and ethanol production in S. cerevisiae
strains expressing bacterial XIs in the presence of E. coli
GroES and GroEL

| | | | Xylose | | Ethanol | |
|---|---|---|---|---|---|---|
| | OD₆₀₀ | | consumed | | Produced | |
| Strain (xylA GeneBank #) | Avg. | SD | Avg. | SD | Avg. | SD |
| After 24 hours of growth | | | | | | |
| Actinoplanes missouriensis | 4.26 | 0.28 | 1.07 | 0.11 | 0.00 | 0.00 |
| (YP_005460771) | | | | | | |
| Burk. phytofirmans PsJN | 8.24 | 1.02 | 13.22 | 4.21 | 5.16 | 1.85 |
| (YP_001890302) | | | | | | |
| Burkholderia phymatum | 7.28 | 0.44 | 9.31 | 0.60 | 3.30 | 0.19 |
| (YP_001858563) | | | | | | |
| Citrobacter youngae | 10.49 | 1.11 | 26.00 | 3.49 | 10.32 | 1.36 |
| (ZP_06571492) | | | | | | |
| E. blattae (YP_006317764) | 9.66 | 1.33 | 24.16 | 1.02 | 9.41 | 0.51 |
| E. coli MG1655 | 9.21 | 0.89 | 17.94 | 4.69 | 6.91 | 1.95 |
| (NP_418022) | | | | | | |
| Pseudomonas fluorescens | 4.47 | 0.36 | 2.28 | 0.24 | 0.00 | 0.00 |
| (EIK60355) | | | | | | |
| Photobacterium profundum | 4.10 | 0.53 | 2.39 | 0.08 | 0.00 | 0.00 |
| (YP_128690) | | | | | | |
| Pantoea stewartii DC283 | 7.52 | 0.59 | 13.92 | 2.03 | 5.18 | 0.80 |
| (ZP_09830211) | | | | | | |
| Plautia stali symbiont | 6.75 | 1.35 | 11.19 | 2.35 | 3.96 | 0.97 |
| (ZP_0825515) | | | | | | |
| Pseud. syringae | 6.13 | 1.15 | 11.81 | 1.10 | 4.29 | 0.43 |
| (ZP_03398764) | | | | | | |
| Vibrio sp. XY-214 | 4.88 | 0.89 | 7.26 | 2.18 | 2.37 | 0.92 |
| (BAI23199) | | | | | | |
| Y. regensburgei | 9.42 | 0.48 | 24.14 | 1.50 | 8.96 | 0.24 |
| (ZP_09387709) | | | | | | |
| After 48 hours of growth | | | | | | |
| Actinoplanes missouriensis | 5.68 | 0.72 | 4.36 | 0.50 | 0.36 | 0.32 |
| (YP_005460771) | | | | | | |
| Burk. phytofirmans PsJN | 12.14 | 0.30 | 40.00 | 0.00 | 16.44 | 0.16 |
| (YP_001890302) | | | | | | |
| Burkholderia phymatum | 11.86 | 0.25 | 38.78 | 0.51 | 15.08 | 0.32 |
| (YP_001858563) | | | | | | |
| Citrobacter youngae | 12.63 | 0.08 | 40.00 | 0.00 | 16.45 | 0.14 |
| (ZP_06571492) | | | | | | |
| E. blattae (YP_006317764) | 12.94 | 0.08 | 40.00 | 0.00 | 16.50 | 0.01 |
| E. coli MG1655 | 12.63 | 0.19 | 40.00 | 0.00 | 16.19 | 0.41 |
| (NP_418022) | | | | | | |
| Pseudomonas fluorescens | 7.55 | 0.82 | 13.45 | 0.95 | 4.27 | 0.39 |
| (EIK60355) | | | | | | |
| Photobacterium profundum | 9.01 | 0.16 | 19.77 | 0.38 | 7.05 | 0.17 |
| (YP_128690) | | | | | | |
| Pantoea stewartii DC283 | 10.90 | 1.63 | 40.00 | 0.00 | 16.38 | 0.07 |
| (ZP_09830211) | | | | | | |
| Plautia stali symbiont | 12.21 | 0.65 | 40.00 | 0.00 | 15.62 | 0.30 |
| (ZP_0825515) | | | | | | |
| Pseud. syringae | 10.22 | 1.33 | 40.00 | 0.00 | 16.47 | 0.03 |
| (ZP_03398764) | | | | | | |
| Vibrio sp. XY-214 | 10.45 | 1.65 | 35.95 | 4.62 | 13.53 | 2.05 |
| (BAI23199) | | | | | | |
| Y. regensburgei | 11.45 | 1.53 | 40.00 | 0.00 | 16.35 | 0.32 |
| (ZP_09387709) | | | | | | |
| After 72 hours of growth | | | | | | |
| Actinoplanes missouriensis | 7.29 | 0.88 | 8.52 | 1.39 | 1.80 | 0.57 |
| (YP_005460771) | | | | | | |

### Example 7

### Xylose Isomerase Activities in Yeast in the Presence or Absence of GroES and GroEL

Xylose isomerase enzyme activity was assayed in S. cereivisiae strains expressing CYXI, EBXI, ECXI, PSTXI, or YRXI in the presence or absence of EC GroES and GroEL. Strains described above were used, as well as C52-79 cells transformed with only the xylose isomerase expression plasmids described in Example 6. Transformants were selected as described in Example 4. The cells were grown in SD medium lacking uracil.

Cell breaking buffer was prepared with 10 mM TEA, pH 7.5, 10 mM MgSO₄, 10 mM MnCl₂, 1 mM of DTT, and one tablet of cOmplete Mini, EDTA-free protease inhibitor cocktail (Roche Diagnostics GmbH) in 50 mL total volume. Bead beating tubes were prepared with approximately 1 gram of 400 micron acid washed silica beads (VWR) in a 2 mL screw cap tube. Cell pellets were resuspended in 1 mL of breaking buffer and added to the bead beating tubes. Tubes were stored on ice. Cell breakage was performed using a Minibeadbeater (Biospec Products) using 3×1 minute cycles with chilling of the tubes on ice between cycles. Tubes were centrifuged for 1 min at 15,000 g to pellet large particles and reduce foaming. 600 μL was removed and transferred to a new microcentrifuge tube. The tubes were centrifuged at 15,000 g for one hour at 4° C. 500 μL of the supernatant was transferred to a new microcentrifuge tube and stored on ice. 1:10 dilutions were made by added 30 μL of the extract to 270 μL of breaking buffer. The remaining extract was frozen on dry ice and transferred to the −80° C. freezer while the dilutions were stored on ice until analysis which took place the same day.

A stock xylose isomerase assay solution was prepared by adding the volumes found in Table 16 to a tube which was stored at room temperature. A solution of 0.5 M xylose was also prepared and stored in a separate tube. All chemicals were obtained from Sigma Aldrich. The source of sorbitol dehydrogenase was sheep liver.

TABLE 16
Assay stock solution composition and final
assay concentration of components.

| | volume for | | final concentration | |
|---|---|---|---|---|
| chemical | stock solution | | in assay | |
| 1M TEA, pH 7.5 | 250 | μL | 10 | mM |
| 1M MgSO4 heptahydrate | 250 | μL | 10 | mM |
| 1M MnCl2 | 250 | μL | 10 | mM |
| NADH (1 mg/mL in water) | 5 | mL | 0.28 | mM |
| Sorbitol Dehydrogenase | 2.5 | mL | 1 | U/mL |
| (10 U/mL in water) | | | | |

| | | | |
|---|---|---|---|
| Water | 11.75 | mL | 1 mL assay |
| | | | volume |

A Cary 300 Bio spectrophotometer (Varian) was set-up for a 10 minute assay time and the cuvette block heater was set to 30° C. 800 μL of the assay stock solution was added to a quartz cuvette and inserted into the instrument cuvette holder and the temperature was allowed to equilibrate for 10 minutes. 100 μL of the extract dilution was added to the cuvette and monitoring at A₃₄₀ₙₘ was initiated. This was continued until a stable linear signal was obtained (background) which typically took 2-4 minutes. 100 μL of 0.5M xylose was then added to start the reaction. Monitoring at A₃₄₀ₙₘ continued until a stable linear signal was obtained (signal) which typically took 2-4 minutes.

A protein assay was performed on the same dilutions used for the xylose isomerase activity assay. 25 μL of each sample was added to a microtiter plate in triplicate. Standard BSA protein standards (Thermo Scientific) were also added in triplicate. 280 μL of Coomassie Plus—The Better Bradford Assay Reagent (Thermo Scientific) was added and the plate was equilibrated at room temperature for 15 minutes. The A_{595 nm} was obtained. A trend line with a polynomial fit was used for the standards to calculate the protein concentration for the samples. A sample was determined to have no activity if the slope after addition of xylose was more positive than the background slope. The activity assay results can be seen in Table 17. Two different transformants were assayed for each construction.

TABLE 17
Xylose isomerase activity assay results

| | Sequence | | |
|---|---|---|---|
| Strain (XI | identity to | | |
| accession #) | E. coli xylA | groEL/ES | XI Activity |
| E. coli MG1655 | 100% | with groEL/ES | 0.271 |
| (NP_418022) | | with groEL/ES | 0.207 |
| | | no groEL/ES | 0.007 |
| | | no groEL/ES | No Activity |
| Citrobacter | 93% | with groEL/ES | 0.268 |
| youngae | | with groEL/ES | 0.381 |
| (ZP_06571492) | | no groEL/ES | No Activity |
| | | no groEL/ES | No Activity |
| E. blattae | 88% | with groEL/ES | 0.244 |
| (YP_006317764) | | with groEL/ES | 0.15 |
| | | no groEL/ES | 0.003 |
| | | no groEL/ES | No Activity |
| Y. regensburgei | 92% | with groEL/ES | 0.311 |
| (ZP_09387709) | | with groEL/ES | 0.394 |
| | | no groEL/ES | 0.014 |
| | | no groEL/ES | 0.007 |
| Pseud. syringae | 68% | with groEL/ES | 0.182 |
| (ZP_03398764) | | with groEL/ES | 0.209 |
| | | no groEL/ES | No Activity |
| | | no groEL/ES | No Activity |

### Example 8

### Use of A. missourinesis Chaperones with Xylose Isomerase

The previous examples demonstrate the effectiveness of E. coli GroEL and GroES in improving the folding and in vivo function of various bacterial xylose isomerases when expressed in S. cerevisiae. In this example, chaperonins from Actinoplanes missouriensis were used. Plasmid pRS423 Am 104GroES 550 GroEL (SEQ ID NO:94) was constructed to contain a set of A. missouriensis chaperonins. In this plasmid the groEL nucleic acid fragment (nt 6446 to 8092 in SEQ ID NO:974; also SEQ ID NO:4) that encodes a polypeptide of 550 amino acids was under the control of the ADH promoter (nt 5762 to 6439). The groES nucleic acid fragment that encodes a polypeptide of 104 amino acids (SEQ ID NO:18) was under the control of the GPD (TDH3) promoter (nt 9332 to 8654). Both the expression cassettes were terminated with a bidirectional CYC1 terminator (nt 8101 to 8319).

In order to determine whether expression of this set of chaperonins from A. missouriensis can improve the folding and function of the xylose isomerase, plasmids pHR81 ilv5p xyIA (AMyXI) (Example 6) and pRS423 Am 104GroES 550 GroEL were transformed into yeast strain C52-79 as described in Example 6. The resulting strain was analyzed for growth and ethanol production from xylose. Using the same growth conditions as described in Example 6, the strain used up all of the xylose (40 g/L) in the medium, producing about 16 g of ethanol after 6 days. The result demonstrated that the xylose isomerase from A. missouriensis can be functionally expressed in the presence of the A. missouriensis chaperonins in yeast.

A second set of coding regions in the A. missouriensis genome sequence is annotated as encoding GroEL and GroES. These coding regions were also cloned in the vector pRS423 the same way as the first set of chaperonins, described above. The resulting construct was pRS423 Am 112GroES 540 GroEL (SEQ ID NO:95). In this construct, the GroEL coding region (nt 6446 to 8068 in SEQ ID NO:95; also SEQ ID NO:6) was under the control of the ADH promoter (nt 5762 to 6439). The GroES coding region (nt 8642 to 8034 in SEQ ID NO:95; also SEQ ID NO:20) was under the control of the GPD (TDH3) promoter (nt 9332-8654). The bidirectional CYC1 terminator (nt 8077 to 8295) was placed between these two expression cassettes. To test whether this construct is functional in yeast, plasmids pHR81 ilv5p xyIA (AMyXI) and pRS423 Am 112GroES 540 GroEL were transformed into yeast strain C52-79 as described in Example 6. The resulting strain was analyzed for growth and ethanol production from xylose. Using the same growth conditions as described in Example 6, the strain used very little xylose and no detectable of amount of ethanol was present in the growth medium. It is possible that this set of chaperonins was not expressed in yeast, or the GroEL and GroES were not matched properly. It is also possible that the annotation in the database is incorrect.

### Example 9

### Expression of Xylose Isomerases from a Cow Rumen Metagenomic Library

Additional candidate bacterial xylose isomerases were tested for activity in yeast when expressed with or without GroES and GroEL. These were two polypeptides identified using amino acid sequences of the xylose isomerases from Ruminococcus flavefaciens FD-1 (SEQ ID NO:96) and from Ruminococcus champanellensis 18P13 (SEQ ID NO:97) in a BLAST search against translated open reading frames of the metagenomic database generated from cow rumen (Matthias Hess, et al. Science 331:463-467 (2011)). These two proteins have 77% amino acid identity to each other. No protein sequences were found to have greater than 70% identity to either of these sequences. Two proteins with sequence identities in the range of 59% to 64% were selected for testing and named Ru2 (SEQ ID NO:98) and Ru3 (SEQ ID NO:100. DNA sequences encoding these proteins were designed using codon optimization for expression in S. cerevisiae, and given designations of xyIA (Ru2) (SEQ ID NO:99) and xyIA(Ru3) (SEQ ID NO:101). The designed nucleic acid molecules were synthesized, including a PmeI site just upstream of the start codon and a SfiI site immediately following the stop codon.

The synthesized xyIA coding regions xyIA(Ru2) and xyIA(Ru3) were inserted between PmeI and SfiI sites in pHR81-AMXA creating chimeric genes for expression as described in Example 4. The xyIA(Ru2) vector was named pHR81 ilv5p xyIA(Ru2) and the xyIA(Ru3) vector was named pHR81 Ilv5p xyIA(Ru3). These constructs were transformed into the C52-79 strain (Example 5) with or without pRS423-GELS (Example 1), the plasmid containing ECgroES and ECgroEL expression cassettes, as in Example 6.

Transformed strains were examined for their ability to consume xylose and to convert xylose to ethanol as described in Example 6. Results of analysis after 24 hr of growth are shown in Table 18. Expression of xyIA(Ru2) or xyIA(Ru3) alone without E. coli chaperonins did not enable the yeast strain to consume xylose or convert xylose to ethanol. On the other hand, with the expression of E. coli chaperonins, the yeast strains containing each of these xylose isomerases could consume xylose and convert xylose to ethanol. The result indicates that expression of E. coli chaperonins enables expression of active Ru2 and Ru3 xylose isomerase enzymes in yeast.

TABLE 18
Growth rate, xylose consumption and ethanol production in S. cerevisiae
strains expressing bacterial XIs in the presence or absence of
E. coli GroES and GroEL

| | | | | Xylose | | Ethanol | |
|---|---|---|---|---|---|---|---|
| Strain | | OD₆₀₀ | | consumed | | Produced | |
| xylA | GroESL | average | SD | average | SD | average | SD |
| Ru2 | + | 9.04 | 0.25 | 14.56 | 0.65 | 5.76 | 0.26 |
| Ru3 | + | 8.46 | 0.65 | 12.08 | 3.25 | 4.60 | 1.48 |
| Ru2 | − | 2.41 | 0.86 | 0.60 | 0.19 | 0.00 | 0.00 |
| Ru3 | − | 2.69 | 0.12 | 0.60 | 0.08 | 0.00 | 0.00 |

What is claimed is:

1. A recombinant yeast cell comprising:
a) at least one gene encoding each amino acid sequence of an interacting pair of Group I chaperonin polypeptides; and
b) at least one gene encoding a bacterial xylose isomerase polypeptide;
wherein:
i) the interacting pair of Group I chaperonins are active in the cytosol of the cell;
ii) the bacterial xylose isomerase polypeptide is converted to an active xylose isomerase enzyme; and
iii) the specific activity of the bacterial xylose isomerase enzyme is higher as compared with the specific activity of the same xylose isomerase enzyme expressed in the absence of the interacting pair of Group I chaperonin polypeptides.

2. The yeast cell of claim 1 wherein the at least one gene encoding each amino acid sequence of an interacting pair of Group I chaperonin polypeptides is derived from a bacterium.

3. The yeast cell of claim 1 wherein the xylose isomerase polypeptide is included in the enzyme classification defined by EC 5.3.1.5.

4. The yeast cell of claim 3 wherein the xylose isomerase polypeptide is selected from the group consisting of Class I xylose isomerases and Class II xylose isomerases.

5. The yeast cell of claim 1 wherein the bacterial xylose isomerase is derived from a member of a genus selected from the group consisting of Actinoplanes, Escherichia, Bacillus, Streptomyces, Burkholderia, Citrobacter, Pseudomonas, Photobacterium, Pantoea, Plautia, Vibrio, Yokenella, Bacteroides, Ruminococcus, and Zymomonas.

6. The yeast cell of claim 2 wherein the bacterium is a member of a genus selected from the group consisting of Actinoplanes, Escherichia, Bacillus, Streptomyces, Burkholderia, Citrobacter, Pseudomonas, Photobacterium, Pantoea, Plautia, Vibrio, Yokenella, Bacteroides, Ruminococcus, and Zymomonas.

7. The yeast cell of claim 2 wherein the interacting pair of Group I chaperonin polypeptides comprises a polypeptide selected from the group consisting of GroEL, GroES, Hsp60 and Hsp10.

8. The yeast cell of claim 7 wherein the interacting pair of Group I chaperonin polypeptides is derived from E. coli.

9. The yeast cell of claim 1 wherein the at least one gene of a) and the at least one gene of b) are derived from different organisms.

10. The yeast cell of claim 9 wherein the xylose isomerase specific activity is at least 50% of the specific activity of the cell wherein a) and b) are from the same bacteria.

11. The yeast cell of claim 1 wherein the xylose isomerase specific activity is at least 50% of the xylose isomerase specific activity obtained in yeast cells expressing E. coli GroES and GroEL chaperonins, and E. coli xylose isomerase.

12. The yeast cell of claim 1 wherein the cell has a complete xylose utilization pathway and has the ability to grow on xylose as a sole carbon source.

13. The yeast cell of claim 12 further comprising a target compound.

14. The yeast cell of claim 13 wherein the target compound is selected from the group consisting of ethanol, butanol, and 1,3-propanediol.

15. A method for producing a yeast strain that has xylose isomerase activity comprising:
a) providing a yeast cell;
b) introducing a heterologous nucleic acid molecule encoding a GroEL polypeptide and a heterologous nucleic acid molecule encoding a GroES polypeptide; and
c) introducing a heterologous nucleic acid molecule encoding a bacterial xylose isomerase polypeptide;
wherein:
i) the GroEL and GroES polypeptides are expressed in the cytosol of the cell;
ii) the xylose isomerase polypeptide is converted to an active xylose isomerase enzyme; and
iii) the specific activity of the xylose isomerase enzyme is higher as compared with the specific activity of the same xylose isomerase enzyme expressed in the absence of the GroEL and GroES polypeptides.

16. A method for expressing an active bacterial xylose isomerase enzyme in yeast comprising:
a) providing a recombinant yeast cell of claim 1; and
b) growing the yeast cell of a) whereby the xylose isomerase polypeptide is converted to an active xylose isomerase enzyme.

17. The method of claim 16 wherein the recombinant yeast cell of (a) further comprises a complete xylose utilization pathway and growing of (b) is in a medium comprising xylose as a carbon source.

18. The method of claim 17 wherein the yeast cell comprises a metabolic pathway that produces a target compound.

19. The method of claim 18 wherein the target compound is selected from the group consisting of ethanol, butanol, and 1,3-propanediol.
